SEQUENCE LISTING (2 sequences)

SEQ ID NO: 1 - protein, 15 aa, Artificial Sequence
  Completely Synthetic Amino Acid Sequence
RSRSRSRSRSRSRSR

SEQ ID NO: 2 - protein, 11 aa, Artificial Sequence
  Completely Synthetic Amino Acid Sequence
AKRRRLSSLRA

(19) United States
(12) Patent Application Publication
(10) Pub. No.: US 2011/0183975 A1
(43) Pub. Date: Jul. 28, 2011

(54) NOVEL 6-AZAINDOLE AMINOPYRIMIDINE DERIVATIVES HAVING NIK INHIBITORY ACTIVITY

(76) Inventors: Yasuhiro Goto, Ibaraki (JP); Takeshi Sagara, Ibaraki (JP); Weiming Fan, Chapel Hill, NC (US); Thomas F. N. Hacell, Morrisville, NC (US); Matthew G. Jenks, Durham, NC (US); Michael J. Malaska, Chapel Hill, NC (US); Joseph A. Moore, III, Raleigh, NC (US); Gilles Ouvry, Reims (FR); Bharathi Pandi, Cary, NC (US); Michael R. Peel, Chapel Hill, NC (US); Kimberty M. Steward, Durham, NC (US)

(21) Appl. No.: 13/122,666

(22) PCT Filed: Sep. 28, 2009
(86) PCT No.: PCT/US09/58561

§ 371 Date: Apr. 5, 2011

Related U.S. Application Data
(60) Provisional application No. 61/195,409, filed on Oct. 7, 2008.

(51) Int. Cl. A61K 31/5377; C07D 471/04; A61K 31/506; A61P 35/00

(52) U.S. Cl. 514/234.5; 544/331; 514/275; 514/252.18; 544/122

(57) ABSTRACT

The present invention relates to a compound of formula I: wherein: R¹ is C₁₋₆ alkyl, C₃₋₈ cycloalkyl, aryl, heterocyclyl, or —COR^{1x}, where the C₁₋₆ alkyl, C₃₋₈ cycloalkyl, aryl, and heterocyclyl may be substituted; and R^{1x} is C₃₋₈ cycloalkyl, aryl, or heterocyclyl, any of which may be substituted; R², R³, R⁴, R⁵, R⁶, and R⁷ are each independently hydrogen, halogen, C₁₋₆ alkyl, or aryl, where the C₁₋₆ alkyl or aryl may be substituted; R⁸ is hydrogen, C₁₋₆ alkyl, aryl, or heterocyclyl, any of which may be substituted; or a pharmaceutically acceptable salt or ester thereof.

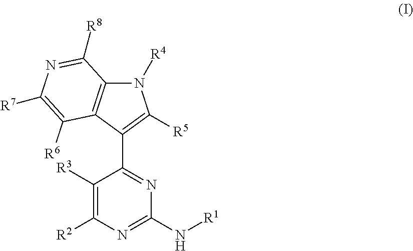

### BACKGROUND OF THE INVENTION

The present invention relates to novel 6-azaindole aminopyrimidine derivatives which are useful in the pharmaceutical field, and more particularly, to those which show potent NIK inhibitory activity, leading to an antitumor or anti-cancer effect, and also to a NIK inhibitor or an antitumor agent containing them.

Nuclear factor-kappa B (NF-kappa B) is a transcription factor regulating the expression of various genes involved in the immune response, cell proliferation, apoptosis, and carcinogenesis [J.C.I., No. 107, pp. 3-6 (2001), Cell, No. 109, pp. S81-S96 (2002)]. NF-kappa B dependent transcriptional activation is tightly controlled by signaling molecules via sequential phosphorylation and protein degradation [Gene & Development., No. 18, pp. 2195-2224 (2004)]. NF-kappa B inducing kinase (NIK, also known as MAP3K14) is a serine/threonine kinase which regulates NF-kappa B pathway activation. NIK phosphorylates IkappaB kinase (IKK) which leads to degradation of IkappaB proteins and activation of NF-kappa B transcription factors. NIK is also known to induce p100 processing by stimulating site specific phosphorylation and ubiquitination of this precursor protein [Molecular Cell, No. 7, pp. 401-409 (2001)].

NIK is an essential upstream kinase for lymphotoxin-beta receptor-induced NF-kappa B pathway activation, which is classified as one of the non-canonical NF-kappa B pathways [Science, No. 291, pp. 2162-2165 (2001)]. Deregulation of non-canonical NF-kappa B pathway is known to link to inflammatory disorders. NIK deficient mice demonstrated resistance to antigen induced arthritis and showed less periarticular osteoclastogenesis and less bone erosion [J.C.I., No. 115, pp. 1848-1854 (2005)], suggesting that NIK is an attractive target for development of anti-rheumatic agents.

It is well known that NF-kappa B is constitutively activated in various types of tumors [Biochemical Pharmacology, No. 72, pp. 1142-1152 (2006)] and is believed to participate in many aspects of oncogenesis via promoting cell growth and preventing apoptosis. Indeed, NIK silencing inhibited proliferation of multiple myeloma cells with high NIK expression due to its gene translocation/amplification [Cancer Cell, No. 12, pp. 115-130 (2007)]. NIK is reported to transform rat fibroblasts. Depletion of NIK in adult T-cell leukemia cells suppressed tumor formation in immunodeficient mice [Blood, No. 15, pp. 5118-5129 (2008)]. These findings indicate that NIK is a potential target for development of anti-cancer agents.

Therefore, NIK inhibitors are considered to be valuable for the treatment of inflammatory disorders and cancer therapy. There have been reported 7-azaindole aminopyrimidine derivatives as tyrosine kinase inhibitors in WO2007/149427, WO2007/107221, etc. However, no 6-azaindole aminopyrimidine derivatives having NIK inhibitory activity have been reported so far.

### DETAILED DESCRIPTION OF THE INVENTION

The purpose of the present invention is to provide novel 6-azaindole aminopyrimidine derivatives which show potent NIK inhibitory activity.

In order to attain such purpose, the present inventors have synthesized a variety of novel 6-azaindole aminopyrimidine derivatives and found that the compound represented by the following Formula I shows good NIK inhibitory activity in an in vitro enzyme and/or cell-based assay.

Thus, the invention relates to a compound of Formula I:

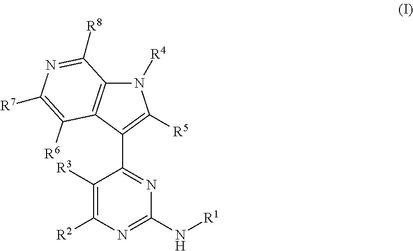

wherein:

R¹ is C₁₋₆ alkyl, C₃₋₈ cycloalkyl, aryl, heterocyclyl, or —COR^{1x},
where the C₁₋₆ alkyl, C₃₋₈ cycloalkyl, aryl, and heterocyclyl may be substituted; and R^{1x} is C₃₋₈ cycloalkyl, aryl, or heterocyclyl, any of which may be substituted;

R², R³, R⁴, R⁵, R⁶, and R⁷ are each independently hydrogen, halogen, C₁₋₆ alkyl, or aryl,
where the C₁₋₆ alkyl or aryl may be substituted;

R⁸ is hydrogen, C₁₋₆ alkyl, aryl, or heterocyclyl, any of which may be substituted;

or a pharmaceutically acceptable salt or ester thereof.

Another embodiment of the present invention is illustrated by a compound of Formula I wherein:

R¹ is aryl, heterocyclyl, or —COR^{1x},

where R^{1x} is aryl or heterocyclyl; and

the aryl or heterocyclyl of R¹ and R^{1x} each independently may be substituted with one or more of the same or different substituents selected from:
(i) a substituent selected from L₁;
(ii) C₁₋₆ alkyl which may be substituted with one or more of the same or different substituents selected from L₂;
(iii) —C₀₋₆ alkylene-aryl;
(iv) —C₀₋₆ alkylene-heterocyclyl;
(v) —OR^{a1};
(vi) —O-J-R^{a2};
(vii) —SR^{a1};
(viii) —S-J-R^{a2};
(ix) —NR^{a3}R^{a4};
(x) —CONR^{a3}R^{a4};
(xi) —SO₂R^{a5};
(xii) —SO₂NR^{a3}R^{a4}; and
(xiii) —COOR^{a5} where:
   (a1) the —C₀₋₆ alkylene-aryl and —C₀₋₆ alkylene-heterocyclyl each independently may be substituted with one or more of the same or different substituents selected from:
      (a1-1) a substituent selected from L₂; and
      (a1-2) C₁₋₆ alkyl which may be substituted with one or more of the same or different substituents selected from L₂;
   (a2) J is —(CR^{y1}R^{y2})—, —(CR^{y1}R^{y2})—(CR^{y3}R^{y4})—, or —(CR^{y1}R^{y2})—(CR^{y3}R^{y4})—(CR^{y5}R^{y6})—,
      where R^{y1}, R^{y2}, R^{y3}, R^{y4}, R^{y5}, and R^{y6} are each independently hydrogen, halogen, hydroxy, cyano, or C₁₋₃ alkyl;
   (a3) R^{a1}, R^{a2}, and R^{a5} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
      where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
      (a3-1) a substituent selected from L₂; and
      (a3-2) C₁₋₆ alkyl which may be substituted with one or more of the same or different substituents selected from L₂;
      (a3-3) —C₀₋₆ alkylene-aryl which may be substituted with one or more of the same or different substituents selected from L₂;
      (a3-4) —C₀₋₆ alkylene-heterocyclyl which may be substituted with one or more of the same or different substituents selected from L₂;
      (a4-1) R^{a3} and R^{a4} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
      where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
         (a4-1-1) a substituent selected from L₂; and
         (a4-1-2) C₁₋₆ alkyl which may be substituted with one or more of the same or different substituents selected from L₂;
         (a4-1-3) —C₀₋₆ alkylene-aryl which may be substituted with one or more of the same or different substituents selected from L₂;
         (a4-1-4) —C₀₋₆ alkylene-heterocyclyl which may be substituted with one or more of the same or different substituents selected from L₂;
      (a4-2) or alternatively, R^{a3} and R^{a4}, together with the nitrogen atom which they are attached to, may form a 5-membered or 6-membered heterocycle,
      where the heterocycle is selected from pyrrolidinyl, piperidinyl, piperazinyl, and morpholinyl, and may be substituted with one or more of the same or different substituents selected from L₃;

R², R³, R⁴, R⁵, R⁶, and R⁷ are each independently hydrogen, halogen, C₁₋₆ alkyl, or aryl,

where the C₁₋₆ alkyl or aryl each independently may be substituted with one or more of the same or different substituents selected from L₂;

R⁸ is hydrogen, C₁₋₆ alkyl, aryl, or heterocyclyl,

where the C₁₋₆ alkyl, aryl, and heterocyclyl each independently may be substituted with one or more of the same or different substituents selected from L₂;

L₁ is halogen, cyano, or nitro;

L₂ is halogen, hydroxy, nitro, cyano, amino, carbamoyl, aminosulfonyl, imino, C₁₋₆ alkylamino, di-(C₁₋₆ alkyl)amino, C₁₋₆ alkylsulfonyl, C₁₋₆ alkylsulfonylamino, C₁₋₆ alkoxy, C₁₋₆ alkoxycarbonyl, C₁₋₆ alkoxycarbonylamino, C₁₋₆ alkanoyl, C₁₋₆ alkanoylamino, C₁₋₆ alkanoyloxy, C₁₋₆ alkylthio, or carboxyl; and

L₃ is halogen, hydroxy, or amino

The invention also relates to a pharmaceutical composition or preparation comprising, together with a pharmaceutically acceptable carrier or diluent, a compound represented by the Formula I or a pharmaceutically acceptable salt or ester thereof.

The invention further relates to a pharmaceutical composition or preparation comprising, together with a pharmaceutically acceptable carrier or diluent, a compound represented by the Formula I or a pharmaceutically acceptable salt or ester thereof, in combination with an antitumor agent selected from the group consisting of antitumor alkylating agents, antitumor antimetabolites, antitumor antibiotics, plant-derived antitumor agents, antitumor platinum coordination compounds, antitumor camptothecin derivatives, antitumor tyrosine kinase inhibitors, monoclonal antibodies, biological response modifiers, and other antitumor agents or a pharmaceutically acceptable salt or ester thereof.

The invention further relates to a method for the treatment of cancer, comprising administering to a patient in need thereof a therapeutically effective amount of a compound represented by the Formula I or a pharmaceutically acceptable salt or ester thereof.

The invention still further relates to a method for the treatment of cancer, comprising administering to a patient in need thereof simultaneously, separately or sequentially a therapeutically effective amount of a compound represented by the Formula I or a pharmaceutically acceptable salt or ester thereof in combination with a therapeutically effective amount of an antitumor agent selected from the group consisting of antitumor alkylating agents, antitumor antimetabolites, antitumor antibiotics, plant-derived antitumor agents, antitumor platinum coordination compounds, antitumor camptothecin derivates, antitumor tyrosine kinase inhibitors, monoclonal antibodies, interferons, biological response modifiers, and other antitumor agents or a pharmaceutically acceptable salt or ester thereof

Furthermore, the invention relates to the use of a NIK inhibitor for the manufacture of a medicament for the treatment of cancer; and the use of a NIK inhibitor in combination with an antitumor agent for the manufacture of a medicament for the treatment of cancer. The invention further relates to a method of treating cancer which comprises administering to a patient in need thereof a therapeutically effective amount of a NIK inhibitor; and a method of treating cancer which comprises administering to a patient in need thereof a therapeutically effective amount of a NIK inhibitor in combination with a therapeutically effective amount of an antitumor agent. The invention still further relates to a pharmaceutical composition or preparation comprising as active ingredient a NIK inhibitor; and a pharmaceutical composition or preparation comprising as active ingredient a NIK inhibitor, together with an antitumor agent.

Embodiments of the compound represented by the Formula I will be illustrated in more detail.

In an embodiment of the compound of the Formula I, R¹ is C₁₋₆ alkyl, C₃₋₈ cycloalkyl, aryl, heterocyclyl, or —COR^{1x}, where the C₁₋₆ alkyl, C₃₋₈ cycloalkyl, aryl, and heterocyclyl may be substituted; and R^{1x} is C₃₋₈ cycloalkyl, aryl or heterocyclyl, any of which may be substituted.

In another embodiment of the compound of the Formula I, R¹ is aryl, heterocyclyl or —COR^{1x},

where R^{1x} is aryl or heterocyclyl; and

the aryl or heterocyclyl of R¹ and R^{1x} each independently may be substituted with one or more of the same or different substituents selected from:
(i) a substituent selected from L₁;
(ii) C₁₋₆ alkyl which may be substituted with one or more of the same or different substituents selected from L₂;
(iii) —C₀₋₆ alkylene-aryl;
(iv) —C₀₋₆ alkylene-heterocyclyl;
(v) —OR^{a1};
(vi) —O-J-R^{a2};
(vii) —SR^{a1};
(viii) —S-J-R^{a2};
(ix) —NR^{a3}R^{a4};
(x) —CONR^{a3}R^{a4};
(xi) —SO₂R^{a5};
(xii) —SO₂NR^{a3}R^{a4}; and
(xiii) —COOR^{a5} where:
   (a1) the —C₀₋₆ alkylene-aryl and —C₀₋₆ alkylene-heterocyclyl each independently may be substituted with one or more of the same or different substituents selected from:
      (a1-1) a substituent selected from L₂; and
      (a1-2) C₁₋₆ alkyl which may be substituted with one or more of the same or different substituents selected from L₂;
   (a2) J is —(CR^{y1}R^{y2})—, —(CR^{y1}R^{y2})—(CR^{y3}R^{y4})— or —(CR^{y1}R^{y2})—(CR^{y3}R^{y4})—(CR^{y5}R^{y6})—,
      where R^{y1}, R^{y2}, R^{y3}, R^{y4}, R^{y5}, and R^{y6} are each independently hydrogen, halogen, hydroxy, cyano, or C₁₋₃ alkyl;
   (a3) R^{a1}, R^{a2}, and R^{a5} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
      where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
      (a3-1) a substituent selected from L₂; and
      (a3-2) C₁₋₆ alkyl which may be substituted with one or more of the same or different substituents selected from L₂;
      (a3-3) —C₀₋₆ alkylene-aryl which may be substituted with one or more of the same or different substituents selected from L₂;
      (a3-4) —C₀₋₆ alkylene-heterocyclyl which may be substituted with one or more of the same or different substituents selected from L₂;
      (a4-1) R^{a1} and R^{a4} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
      where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
         (a4-1-1) a substituent selected from L₂; and
         (a4-1-2) C₁₋₆ alkyl which may be substituted with one or more of the same or different substituents selected from L₂;
         (a4-1-3) —C₀₋₆ alkylene-aryl which may be substituted with one or more of the same or different substituents selected from L₂;
         (a4-1-4) —C₀₋₆ alkylene-heterocyclyl which may be substituted with one or more of the same or different substituents selected from L₂;
      (a4-2) or alternatively, R^{a3} and R^{a4}, together with the nitrogen atom which they are attached to, may form a 5-membered or 6-membered heterocycle,
      where the heterocycle is selected from pyrrolidinyl, piperidinyl, piperazinyl, and morpholinyl, and may be substituted with one or more of the same or different substituents selected from L₃;

R², R³, R⁴, R⁵, R⁶, and R⁷ are each independently hydrogen, halogen, C₁₋₆ alkyl, or aryl,

where the C₁₋₆ alkyl or aryl each independently may be substituted with one or more of the same or different substituents selected from L₂;

R⁸ is hydrogen, C₁₋₆ alkyl, aryl, or heterocyclyl,

where the C₁₋₆ alkyl, aryl, and heterocyclyl each independently may be substituted with one or more of the same or different substituents selected from L₂;

L₁ is halogen, cyano, or nitro;

L₂ is halogen, hydroxy, nitro, cyano, amino, carbamoyl, aminosulfonyl, imino, C₁₋₆ alkylamino, di-(C₁₋₆ alkyl)amino, C₁₋₆ alkylsulfonyl, C₁₋₆ alkylsulfonylamino, C₁₋₆ alkoxy, C₁₋₆ alkoxycarbonyl, C₁₋₆ alkoxycarbonylamino, C₁₋₆ alkanoyl, C₁₋₆ alkanoylamino, C₁₋₆ alkanoyloxy, C₁₋₆ alkylthio, or carboxyl; and

L₃ is halogen, hydroxy, or amino

In another embodiment of the compound of the Formula I, R¹ is phenyl, or heterocyclyl selected from furanyl, pyrrolyl, thienyl, pyridinyl, pyrazinyl, pyridazinyl, pyrimidinyl, thiazolyl, isothiazolyl, oxazolyl, oxadiazolyl, isoxazolyl, imidazolyl, pyrazolyl, and triazolyl, any of which may be substituted with one to three of the same or different substituents selected from:
(i) a substituent selected from L₁;
(ii) C₁₋₆ alkyl which may be substituted with one to three of the same or different substituents selected from L₂ₐ;
(iii) phenyl;
(iv) —C₁₋₃ alkylene-heterocyclyl, the heterocyclyl of which is selected from pyrrolidinyl, piperidinyl, piperazinyl, and morpholinyl;
(v) —OR^{a1};
(vi) —SR^{a1};
(vii) —NR^{a3}R^{a4};
(viii) —CONR¹³R^{a4};
(ix) —SO₂NR^{a3}R^{a4}; and
(x) —COOR^{a5} where:
   (aa1) the phenyl and —C₁₋₃ alkylene-heterocyclyl each independently may be substituted with one to three of the same or different substituents selected from:
      (aa1-1) a substituent selected from L_{2b}; and
      (aa1-2) C₁₋₆ alkyl which may be substituted with one to three of the same or different substituents selected from L_{2b};
   (aa2) R^{a1} and R^{a5} are each independently hydrogen; C₁₋₆ alkyl which may be substituted with one to three of the same or different substituents selected from L_{2b}; or —C₀₋₆ alkylene-phenyl which may be substituted with one to three of the same or different substituents selected from:
   (aa2-1) a substituent selected from L_{2b}; and
   (aa2-2) C₁₋₆ alkyl which may be substituted with one to three of the same or different substituents selected from L_{2b};
   (aa3-1) R^{a3 and R}^{a4} are each independently hydrogen; C₁₋₆ alkyl which may be substituted with one to three of the same or different substituents selected from L_{2c}; —C₀₋₆ alkylene-C₅₋₆ cycloalkyl; —C₁₋₆ alkylene-phenyl; and —C₁₋₆ alkylene-heterocyclyl,
   where the heterocyclyl of —C₁₋₆ alkylene-heterocyclyl is a 5-membered or 6-membered aliphatic or aromatic heterocycle, and
   the —C₀₋₆ alkylene-C₅₋₆ cycloalkyl, —C₁₋₆ alkylene-aryl, and —C₁₋₆ alkylene-heterocyclyl each independently may be substituted with one to three of the same or different substituents selected from:
      (aa3-1-1) a substituent selected from L_{2c}; and
      (aa3-1-2) C₁₋₆ alkyl which may be substituted with one to three of the same or different substituents selected from L_{2c};
   (aa3-2) or alternatively, R^{a3} and R^{a4}, together with the nitrogen atom which they are attached to, may form a 5-membered or 6-membered aliphatic heterocycle, where the aliphatic heterocycle is selected from pyrrolidinyl, piperidinyl, piperazinyl, and morpholinyl, and may be substituted with one to three of the same or different substituents selected from L₃;

L₂ₐ is halogen, hydroxy, carbamoyl, or carboxyl;

L_{2b} is halogen or hydroxy;

L_{2c} is halogen, hydroxy, cyano, amino, carbamoyl, C₁₋₆ alkylamino, di-(C₁₋₆ alkyl)amino, C₁₋₆ alkoxy, C₁₋₆ alkoxycarbonylamino, C₁₋₆ alkanoyl, Cᵢ₋₆ alkanoylamino, C₁₋₆ alkanoyloxy, C₁₋₆ alkylthio, or carboxyl

L₃ is halogen, hydroxy, or amino

In yet another embodiment of the compound of the Formula I, R¹ is phenyl disubstituted at the 2- and 5-positions with C₁₋₆ alkyl or —OR^{a1} and with —CONR^{a3}R^{a4}, respectively, where:
(aaa1) R^{a1} is C₁₋₆ alkyl which may be substituted with one to three of the same or different substituents selected from L_{2b}; or —C₀₋₆ alkylene-phenyl which may be substituted with one to three of the same or different substituents selected from:
   (aaa1-1) a substituent selected from L_{2b}; and
   (aaa1-2) C₁₋₆ alkyl which may be substituted with one to three of the same or different substituents selected from L_{2b};
(aaa2) R^{a3} and R^{a4} are each independently hydrogen; C₁₋₆ alkyl which may be substituted with one to three of the same or different substituents selected from L_{2c}; —C₀₋₃ alkylene-C₅₋₆ cycloalkyl; —C₁₋₃ alkylene-phenyl; or —C₁₋₃ alkylene-heterocyclyl,
   where the heterocyclyl of —C₁₋₃ alkylene-heterocyclyl is selected from pyrrolidinyl, piperidinyl, piperazinyl, morpholinyl, furanyl, pyrrolyl, thienyl, pyridinyl, pyrazinyl, pyridazinyl, pyrimidinyl, thiazolyl, isothiazolyl, oxazolyl, oxadiazolyl, isoxazolyl, and imidazolyl; and the —C₀₋₃ alkylene-C₅₋₆ cycloalkyl; —C₁₋₃ alkylene-phenyl; or —C₁₋₃ alkylene-heterocyclyl each independently may be substituted with one to three of the same or different substituents selected from:
      (aaa2-1) a substituent selected from L_{2c}; and
      (aaa2-2) C₁₋₆ alkyl which may be substituted with one to three of the same or different substituents selected from L_{2c};

L_{2b} is halogen or hydroxy;

L_{2c} is halogen, hydroxy, cyano, amino, carbamoyl, C₁₋₆ alkylamino, di-(C₁₋₆ alkyl)amino, C₁₋₆ alkoxy, C₁₋₆ alkanoylamino, C₁₋₆ alkylthio, or carboxyl.

In the case where R¹ is phenyl disubstituted at the 2- and 5-positions with C₁₋₆ alkyl or —OR^{a1} and with —CONR^{a3}R^{a4}, respectively, the compounds according to the present invention exhibit excellent NIK selective inhibitory activity. In a preferred embodiment, R¹ is phenyl disubstituted at the 2- and 5-positions with C₁₋₆ alkyl or —OR^{a1} and with —CONR^{a3}R^{a4}, where R^{a1} is C₁₋₆ alkyl which may be substituted with one to three of the same or different substituents selected from halogen or hydroxy, and R^{a3} and R^{a4} are each independently hydrogen, or C₁₋₆ alkyl which may be substituted with one to three of the same or different substituents selected from halogen or hydroxy.

In an embodiment of the compound of the Formula I, R², R³, R⁴, R⁵, R⁶ and R⁷ are each independently hydrogen, halogen, C₁₋₆ alkyl, or aryl, where the alkyl or aryl may be substituted.

In another embodiment of the compound of the Formula I, R², R³, R⁵, R⁶, and R⁷ are each hydrogen.

In another embodiment of the compound of the Formula I, R⁴ is C₁₋₆ alkyl.

In yet another embodiment of the compound of the Formula I, R⁴ is isopropyl.

In an embodiment of the compound of the Formula I, R⁸ is hydrogen, C₁₋₆ alkyl, aryl or heterocyclyl, any of which may be substituted.

In another embodiment of the compound of the Formula I, R⁸ is hydrogen.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

It is understood that substituents and substitution patterns on the compounds of the present invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” or “may be substituted with one or more substituents” should be taken to be equivalent to the phrase “optionally substituted with at least one substituent” or “may be substituted with at least one substituent”, respectively, and in such cases another embodiment will have from zero to three substituents.

Next, symbols and terms used in the present specification will be explained.

As used herein, the term “alkyl” is intended to include both branched and straight-chain saturated aliphatic hydrocarbon groups having the specified number of carbon atoms. For example, C₁₋₆, as in the term “C₁₋₆ alkyl” is defined to include groups having 1, 2, 3, 4, 5, or 6 carbons in a linear or branched arrangement. For example, the term “C₁₋₆ alkyl” specifically includes methyl, ethyl, n-propyl, i-propyl, n-butyl, t-butyl, i-butyl, pentyl, hexyl, and so on. Generally, the term “Cₘ₋ₙ alkyl” is defined to include groups having m to n carbons in a linear or branched arrangement, where m and n each independently are an integer of 1 to 6 but n is greater than m.

As used herein, the term “cycloalkyl” means a monocyclic saturated aliphatic hydrocarbon group having the specified number of carbon atoms. For example, the term “C₃₋₈ cycloalkyl” includes cyclopropyl, methyl-cyclopropyl, cyclobutyl, 2,2-dimethyl-cyclobutyl, 2-ethyl-cyclopentyl, cyclohexyl, cyclohexyl, cycloheptyl, cyclooctyl, and so on. In an embodiment of the invention the term “cycloalkyl” includes the groups described immediately above and further includes monocyclic unsaturated aliphatic hydrocarbon groups. For example, the term “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

As used herein, the term “alkylene” means a hydrocarbon diradical group having the specified number of carbon atoms. For example, “C₀₋₆ alkylene” includes a single bond, —CH₂—, —CH₂CH₂—, —CH₂CH₂CH₂—, —CH₂CH₂CH₂CH₂—, —CH₂CH₂CH₂CH₂CH₂—, and —CH₂CH₂CH₂CH₂CH₂CH₂—, any of which may be substituted. And “C₁₋₆ alkylene” includes —CH₂—, —CH₂CH₂—, —CH₂CH₂CH₂—, —CH₂CH₂CH₂CH₂—, —CH₂CH₂CH₂CH₂CH₂— —CH₂CH₂CH₂CH₂CH₂CH₂—, any of which may be substituted.

As used herein, the term “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

In certain instances, substituents may be defined with a range of carbons that includes zero, such as —C₀₋₆ alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, the term “heteroaryl” represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As used herein, the term “heterocycle” or “heterocyclyl” is intended to mean a 3- to 10-membered aromatic or nonaromatic heterocycle containing from 1 to 4 heteroatoms selected from the group consisting of O, N and S, and includes bicyclic groups. For the purposes of this invention, the term “heterocyclic” is also considered to be synonymous with the terms “heterocycle” and “heterocyclyl” and is understood as also having the definitions set forth herein. “Heterocyclyl” therefore includes the above mentioned heteroaryls, as well as dihydro and tetrathydro analogs thereof Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

In an embodiment, the term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” in this embodiment therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As used herein, the term “halogen atom” is intended to include fluorine atom, chlorine atom, bromine atom or iodine atom. Among them, for example, fluorine atom, chlorine atom or bromine atom is preferred.

As used herein, the term “C₁₋₆ alkylamino” means a “C₁₋₆ alkyl” group attached through an amino group where the amino group is N-substituted with the “C₁₋₆ alkyl”, and examples thereof include N-methylamino, N-ethylamino, N-propylamino, N-isopropylamino, N-butylamino, N-isobutylamino, N-tert-butylamino, N-pentylamino and N-hexylamino.

As used herein, the term “di-(C₁₋₆ alkyl)amino” means a “C₁₋₆ alkyl” group attached through an amino group where the amino group is N,N-disubstituted with the “C₁₋₆ alkyl”, and examples thereof include N,N-dimethylamino, N,N-diethylamino, N,N-dipropylamino, N,N-diisopropylamino, N,N-dibutylamino, N,N-diisobutylamino, N,N-di-tert-butylamino, N,N-dipentylamino, N,N-dihexylamino, N-ethyl-N-methylamino and N-methyl-N-propylamino.

As used herein, the term “C₁₋₆ alkylsulfonyl” means a group a “C₁₋₆ alkyl” group attached through a sulfonyl group, and examples thereof include methylsulfonyl, ethylsulfonyl and butylsulfonyl.

As used herein, the term “C₁₋₆ alkylsulfonylamino” means a “C₁₋₆ alkylsulfonyl” group attached through an amino group, and examples thereof include methylsulfonylamino, ethylsulfonylamino and butylsulfonylamino.

As used herein, the term “C₁₋₆ alkoxy” means a “C₁₋₆ alkyl” group attached through an oxygen bridge and examples thereof include methoxy, ethoxy, propoxy, isopropoxy, butoxy, isobutoxy, sec-butoxy, tert-butoxy, pentyloxy, neopentyloxy, hexyloxy and isohexyloxy.

As used herein, the term “C₁₋₆ alkoxycarbonyl” means a “C₁₋₆ alkoxy” group attached through a carbonyl group, and examples thereof include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl, neopentyloxycarbonyl, hexyloxycarbonyl and isohexyloxycarbonyl.

As used herein, the term “C₁₋₆ alkoxycarbonylamino” means a “C₁₋₆ alkoxycarbonyl” attached through an amino group, and examples thereof include methoxycarbonylamino, ethoxycarbonylamino, propoxycarbonylamino, isopropoxycarbonylamino, butoxycarbonylamino, isobutoxycarbonylamino, sec-butoxycarbonylamino, tert-butoxycarbonylamino, pentyloxycarbonylamino, neopentyloxycarbonylamino, hexyloxycarbonylamino and isohexyloxycarbonylamino.

As used herein, the term “C₁₋₆ alkanoyl” means a “C₁₋₆ alkyl” group attached through a carbonyl group, and examples thereof include acetyl, propionyl, butyryl, isobutyryl, valeryl, isovaleryl, pivaloyl and pentanoyl.

As used herein, the term “C₁₋₆ alkanoylamino” means a “C₁₋₆ alkanoyl” group attached through an amino group, and examples thereof include acetylamino, propionylamino, butyrylamino, isobutyrylamino, valerylamino, isovalerylamino, pivaloylamino and pentanoylamino.

As used herein, the term “C₁₋₆ alkanoyloxy” means a “C₁₋₆ alkanoyl” attached through an oxygen bridge, and examples thereof include acetyloxy, propionyloxy, butyryloxy, isobutyryloxy, valeryloxy, isovaleryloxy, pivaloyloxy and pentanoyloxy.

As used herein, the term “C₁₋₆ alkylthio” means a “C₁₋₆ alkyl” group attached through a sulfur bridge, and examples thereof include methylthio, ethylthio and butylthio.

The alkyl, cycloalkyl, aryl, heteroaryl and heterocyclyl substituents may be substituted or unsubstituted, unless specifically defined otherwise. For example, C₁₋₆ alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition:

—(C═O)CH₂CH(OH)CH₃, —(C═O)OH, —CH₂(OH)CH₂CH(O), and so on.

As used herein, the term “NIK selective inhibitor” means a compound or a drug which selectively inhibits NIK over other kinases such as, for example, ROCK2. The term “ROCK” means Rho-associated coiled-coil-containing protein kinase. The “NIK selective inhibitor” is preferably a compound or a drug of which inhibitory activity against NIK is at least 10 times the activity against ROCK2; and more preferably a compound or a drug of which inhibitory activity against NIK are at least 100 times the activity against ROCK2.

Explanation for the term “pharmaceutically acceptable salt of ester thereof” or the term “pharmaceutically acceptable carrier or diluent” as used herein will be given later.

As used herein, the term “treatment of cancer” means inhibition of cancer cell growth by administering an antitumor agent to a cancer patient. Preferably, this treatment enables retrogression of cancer growth, that is, reduction in the measurable cancer size. More preferably, such treatment completely eliminates cancer.

As used herein, the term “cancer” refers to solid cancer and hematopoietic cancer. Here, examples of solid cancer include cerebral tumor, head and neck cancer, esophageal cancer, thyroid cancer, small cell lung cancer, non-small cell lung cancer, breast cancer, stomach cancer, gallbladder and bile duct cancer, liver cancer, pancreas cancer, colon cancer, rectal cancer, ovarian cancer, chorioepithelioma, uterine cancer, cervical cancer, renal pelvic and ureteral cancer, bladder cancer, prostate cancer, penile cancer, testicular cancer, embryonal cancer, wilms tumor, skin cancer, malignant melanoma, neuroblastoma, osteosarcoma, Ewing's tumor and soft tissue sarcoma. On the other hand, examples of hematopoietic cancer include acute leukemia, chronic lymphatic leukemia, chronic myelocytic leukemia, polycythemia vera, malignant lymphoma, multiple myeloma and non-Hodgkins' lymphoma.

As used herein, the term “preparation” may usually comprise a therapeutically effective amount of a compound according to the invention, together with a pharmaceutically acceptable carrier or diluent. This technique of formulation is considered to be a technical common knowledge to those having ordinary skill in the pertinent art and is well known. Preferably, oral preparations, intravenous drip infusions or injections can be prepared in combination with a pharmaceutically acceptable carrier or diluent, by various methods that are well known in the art.

As used herein, the term “administration” refers to parenteral administration and/or oral administration. Thus, when a combined/kit-type preparation is administered, both administrations may be parenteral; one administration may be parenteral while the other may be oral; or both administrations may be oral. As used herein, the term “parenteral administration” is, for example, intravenous administration, subcutaneous administration or intramuscular administration, and preferably it is intravenous administration.

In an embodiment of the present invention, a compound represented by the Formula I may be administered simultaneously with other antitumor agent(s). Further, it is possible to administer the compound represented by the Formula I first and then another antitumor agent consecutively, or alternatively it is possible to administer another antitumor agent first and then the compound represented by the Formula I consecutively. It is also possible to administer the compound represented by the Formula I first and then separately administer another antitumor agent after a while, or alternatively it is possible to administer another antitumor agent first and then separately administer the compound represented by the Formula I after a while. The order and the time interval for the administration may be appropriately selected by a person skilled in the art in accordance with, for example, a preparation containing the compound represented by the Formula I used and a preparation containing an antitumor agent that is used in combination therewith, the type of the cancer cells to be treated and the condition of the patient.

As used herein, the term “antitumor alkylating agent” refers to an alkylating agent having antitumor activity, and the term “alkylating agent” herein generally refers to an agent giving an alkyl group in the alkylation reaction in which a hydrogen atom of an organic compound is substituted with an alkyl group. The term “antitumor alkylating agent” may be exemplified by nitrogen mustard N-oxide, cyclophosphamide, ifosfamide, melphalan, busulfan, mitobronitol, carboquone, thiotepa, ranimustine, nimustine, temozolomide or carmustine.

As used herein, the term “antitumor antimetabolite” refers to an antimetabolite having antitumor activity, and the term “antimetabolite” herein includes, in a broad sense, substances which disturb normal metabolism and substances which inhibit the electron transfer system to prevent the production of energy-rich intermediates, due to their structural or functional similarities to metabolites that are important for living organisms, such as vitamins, coenzymes, amino acids and saccharides. The term “antitumor antimetabolites” may be exemplified methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine or pemetrexed disodium and the like.

As used herein, the term “antitumor antibiotic” refers to an antibiotic having antitumor activity, and the “antibiotic” herein includes substances that are produced by microorganisms or by organic synthesis and inhibit cell growth and other functions of microorganisms and of other living organisms. The term “antitumor antibiotic” may be exemplified by actinomycin D, doxorubicin, daunorubicin, neocarzinostatin, bleomycin, peplomycin, mitomycin C, aclarubicin, pirarubicin, epirubicin, zinostatin stimalamer, idarubicin, sirolimus or valrubicin.

As used herein, the term “plant-derived antitumor agent” includes compounds having antitumor activities which originate from plants, or compounds prepared by applying chemical modification to the foregoing compounds. The term “plant-derived antitumor agent” may be exemplified by vincristine, vinblastine, vindesine, etoposide, sobuzoxane, docetaxel, paclitaxel and vinorelbine.

As used herein, the term “antitumor camptothecin derivative” refers to compounds that are structurally related to camptothecin and inhibit cancer cell growth, including camptothecin per se. The term “antitumor camptothecin derivative” is not particularly limited to, but may be exemplified by, camptothecin, 10-hydroxycamptothecin, topotecan, irinotecan or 9-aminocamptothecin. Further, irinotecan is metabolized in vivo and exhibits antitumor effect as SN-38. The action mechanism and the activity of the camptothecin derivatives are believed to be virtually the same as those of camptothecin (e.g., Nitta, et al., Gan to Kagaku Ryoho, 14, 850-857 (1987)).

As used herein, the term “antitumor platinum coordination (platinum-complex) compound” refers to a platinum coordination compound having antitumor activity, and the term “platinum coordination compound” herein refers to a platinum coordination compound which provides platinum in ion form. Preferred platinum compounds include cisplatin; cis-diamminediaquoplatinum (II)-ion; chloro(diethylenetriamine)-platinum (II) chloride; dichloro(ethylenediamine)-platinum (II); diammine(1,1-cyclobutanedicarboxylato) platinum (II) (carboplatin); spiroplatin; iproplatin; diammine(2-ethylmalonato)platinum (II); ethylenediaminemalonatoplatinum (II); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin. Further, other antitumor platinum coordination compounds mentioned in the specification are known and are commercially available and/or producible by a person having ordinary skill in the art by conventional techniques.

As used herein, the term “antitumor tyrosine kinase inhibitor” refers to a tyrosine kinase inhibitor having antitumor activity, and the term “tyrosine kinase inhibitor” herein refers to a chemical substance inhibiting “tyrosine kinase” which transfers a γ-phosphate group of ATP to a hydroxy group of a specific tyrosine in protein. The term “antitumor tyrosine kinase inhibitor” may be exemplified by gefitinib, imatinib, sorafenib, sunitinib, dasatinib, or erlotinib.

As used herein, the term “monoclonal antibody”, which is also known as single clonal antibody, refers to an antibody produced by a monoclonal antibody-producing cell, and examples thereof include cetuximab, bevacizumab, rituximab, alemtuzumab and trastuzumab.

As used herein, the term “interferon” refers to an interferon having antitumor activity, and it is a glycoprotein having a molecular weight of about 20,000 which is produced and secreted by most animal cells upon viral infection. It has not only the effect of inhibiting viral growth but also various immune effector mechanisms including inhibition of growth of cells (in particular, tumor cells) and enhancement of the natural killer cell activity, thus being designated as one type of cytokine. Examples of “interferon” include interferon α, interferon α-2a, interferon α-2b, interferon β, interferon γ-1a and interferon γ-n1.

As used herein, the term “biological response modifier” is the so-called biological response modifier or BRM and is generally the generic term for substances or drugs for modifying the defense mechanisms of living organisms or biological responses such as survival, growth or differentiation of tissue cells in order to direct them to be useful for an individual against tumor, infection or other diseases. Examples of the “biological response modifier” include krestin, lentinan, sizofiran, picibanil and ubenimex.

As used herein, the term “other antitumor agent” refers to an antitumor agent which does not belong to any of the above-described agents having antitumor activities. Examples of the “other antitumor agent” include mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

The above terms “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent” are all known and are either commercially available or producible by a person skilled in the art by methods known per se or by well-known or conventional methods. The process for preparation of gefitinib is described, for example, in U.S. Pat. No. 5,770,599; the process for preparation of cetuximab is described, for example, in WO 96/40210; the process for preparation of bevacizumab is described, for example, in WO 94/10202; the process for preparation of oxaliplatin is described, for example, in U.S. Pat. Nos. 5,420,319 and 5,959,133; the process for preparation of gemcitabine is described, for example, in U.S. Pat. Nos. 5,434,254 and 5,223,608; and the process for preparation of camptothecin is described in U.S. Pat. Nos. 5,162,532, 5,247,089, 5,191,082, 5,200,524, 5,243,050 and 5,321,140; the process for preparation of irinotecan is described, for example, in U.S. Pat. No. 4,604,463; the process for preparation of topotecan is described, for example, in U.S. Pat. No. 5,734,056; the process for preparation of temozolomide is described, for example, in JP-B No. 4-5029; and the process for preparation of rituximab is described, for example, in JP-W No. 2-503143.

The above-mentioned antitumor alkylating agents are commercially available, as exemplified by the following: nitrogen mustard N-oxide from Mitsubishi Pharma Corp. as Nitromin (tradename); cyclophosphamide from Shionogi & Co., Ltd. as Endoxan (tradename); ifosfamide from Shionogi & Co., Ltd. as Ifomide (tradename); melphalan from GlaxoSmithKline Corp. as Alkeran (tradename); busulfan from Takeda Pharmaceutical Co., Ltd. as Mablin (tradename); mitobronitol from Kyorin Pharmaceutical Co., Ltd. as Myebrol (tradename); carboquone from Sankyo Co., Ltd. as Esquinon (tradename); thiotepa from Sumitomo Pharmaceutical Co., Ltd. as Tespamin (tradename); ranimustine from Mitsubishi Pharma Corp. as Cymerin (tradename); nimustine from Sankyo Co., Ltd. as Nidran (tradename); temozolomide from Schering Corp. as Temodar (tradename); and carmustine from Guilford Pharmaceuticals Inc. as Gliadel Wafer (tradename).

The above-mentioned antitumor antimetabolites are commercially available, as exemplified by the following: methotrexate from Takeda Pharmaceutical Co., Ltd. as Methotrexate (tradename); 6-mercaptopurine riboside from Aventis Corp. as Thioinosine (tradename); mercaptopurine from Takeda Pharmaceutical Co., Ltd. as Leukerin (tradename); 5-fluorouracil from Kyowa Hakko Kogyo Co., Ltd. as 5-FU (tradename); tegafur from Taiho Pharmaceutical Co., Ltd. as Futraful (tradename); doxyfluridine from Nippon Roche Co., Ltd. as Furutulon (tradename); carmofur from Yamanouchi Pharmaceutical Co., Ltd. as Yamafur (tradename); cytarabine from Nippon Shinyaku Co., Ltd. as Cylocide (tradename); cytarabine ocfosfate from Nippon Kayaku Co., Ltd. as Strasid(tradename); enocitabine from Asahi Kasei Corp. as Sanrabin (tradename); S-1 from Taiho Pharmaceutical Co., Ltd. as TS-1 (tradename); gemcitabine from Eli Lilly & Co. as Gemzar (tradename); fludarabine from Nippon Schering Co., Ltd. as Fludara (tradename); and pemetrexed disodium from Eli Lilly & Co. as Alimta (tradename).

The above-mentioned antitumor antibiotics are commercially available, as exemplified by the following: actinomycin D from Banyu Pharmaceutical Co., Ltd. as Cosmegen (tradename); doxorubicin from Kyowa Hakko Kogyo Co., Ltd. as adriacin (tradename); daunorubicin from Meiji Seika Kaisha Ltd. as Daunomycin; neocarzinostatin from Yamanouchi Pharmaceutical Co., Ltd. as Neocarzinostatin (tradename); bleomycin from Nippon Kayaku Co., Ltd. as Bleo (tradename); pepromycin from Nippon Kayaku Co, Ltd. as Pepro (tradename); mitomycin C from Kyowa Hakko Kogyo Co., Ltd. as Mitomycin (tradename); aclarubicin from Yamanouchi Pharmaceutical Co., Ltd. as Aclacinon (tradename); pirarubicin from Nippon Kayaku Co., Ltd. as Pinorubicin (tradename); epirubicin from Pharmacia Corp. as Pharmorubicin (tradename); zinostatin stimalamer from Yamanouchi Pharmaceutical Co., Ltd. as Smancs (tradename); idarubicin from Pharmacia Corp. as Idamycin (tradename); sirolimus from Wyeth Corp. as Rapamune (tradename); and valrubicin from Anthra Pharmaceuticals Inc. as Valstar (tradename).

The above-mentioned plant-derived antitumor agents are commercially available, as exemplified by the following: vincristine from Shionogi & Co., Ltd. as Oncovin (tradename); vinblastine from Kyorin Pharmaceutical Co., Ltd. as Vinblastine (tradename); vindesine from Shionogi & Co., Ltd. as Fildesin (tradename); etoposide from Nippon Kayaku Co., Ltd. as Lastet (tradename); sobuzoxane from Zenyaku Kogyo Co., Ltd. as Perazolin (tradename); docetaxel from Aventis Corp. as Taxsotere (tradename); paclitaxel from Bristol-Myers Squibb Co. as Taxol (tradename); and vinorelbine from Kyowa Hakko Kogyo Co., Ltd. as Navelbine (tradename).

The above-mentioned antitumor platinum coordination compounds are commercially available, as exemplified by the following: cisplatin from Nippon Kayaku Co., Ltd. as Randa (tradename); carboplatin from Bristol-Myers Squibb Co. as Paraplatin (tradename); nedaplatin from Shionogi & Co., Ltd. as Aqupla (tradename); and oxaliplatin from Sanofi-Synthelabo Co. as Eloxatin (tradename).

The above-mentioned antitumor camptothecin derivatives are commercially available, as exemplified by the following: irinotecan from Yakult Honsha Co., Ltd. as Campto (tradename); topotecan from GlaxoSmithKline Corp. as Hycamtin (tradename); and camptothecin from Aldrich Chemical Co., Inc., U.S.A.

The above-mentioned antitumor tyrosine kinase inhibitors are commercially available, as exemplified by the following: gefitinib from AstraZeneca Corp. as Iressa (tradename); imatinib from Novartis AG as Gleevec (tradename); sorafenib from Bayer as Nexavar (tradename); sunitinib from Pfizer as Sutent (tradename); dasatinib from Bristol Myers Squibb as Sprycel (tradename); and erlotinib from OSI Pharmaceuticals Inc. as Tarceva (tradename).

The above-mentioned monoclonal antibodies are commercially available, as exemplified by the following: cetuximab from Bristol-Myers Squibb Co. as Erbitux (tradename); bevacizumab from Genentech, Inc. as Avastin (tradename); rituximab from Biogen Idec Inc. as Rituxan (tradename); alemtuzumab from Berlex Inc. as Campath (tradename); and trastuzumab from Chugai Pharmaceutical Co., Ltd. as Herceptin (tradename).

The above-mentioned interferons are commercially available, as exemplified by the following: interferon a from Sumitomo Pharmaceutical Co., Ltd. as Sumiferon (tradename); interferon α-2a from Takeda Pharmaceutical Co., Ltd. as Canferon-A (tradename); interferon α-2b from Schering-Plough Corp. as Intron A (tradename); interferon β from Mochida Pharmaceutical Co., Ltd. as IFNβ (tradename); interferon γ-1a from Shionogi & Co., Ltd. as Imunomax-γ (tradename); and interferon γ-n1 from Otsuka Pharmaceutical Co., Ltd. as Ogamma (tradename).

The above-mentioned biological response modifiers are commercially available, as exemplified by the following: krestin from Sankyo Co., Ltd. as krestin (tradename); lentinan from Aventis Corp. as Lentinan (tradename); sizofiran from Kaken Seiyaku Co., Ltd. as Sonifiran (tradename); picibanil from Chugai Pharmaceutical Co., Ltd. as Picibanil (tradename); and ubenimex from Nippon Kayaku Co., Ltd. as Bestatin (tradename).

The above-mentioned other antitumor agents are commercially available, as exemplified by the following: mitoxantrone from Wyeth Lederle Japan, Ltd. as Novantrone (tradename); L-asparaginase from Kyowa Hakko Kogyo Co., Ltd. as Leunase (tradename); procarbazine from Nippon Roche Co., Ltd. as Natulan (tradename); dacarbazine from Kyowa Hakko Kogyo Co., Ltd. as Dacarbazine (tradename); hydroxycarbamide from Bristol-Myers Squibb Co. as Hydrea (tradename); pentostatin from Kagaku Oyobi Kessei Ryoho Kenkyusho as Coforin (tradename); tretinoin from Nippon Roche Co., Ltd. As Vesanoid (tradename); alefacept from Biogen Idec Inc. as Amevive (tradename); darbepoetin alfa from Amgen Inc. as Aranesp (tradename); anastrozole from AstraZeneca Corp. as Arimidex (tradename); exemestane from Pfizer Inc. as Aromasin (tradename); bicalutamide from AstraZeneca Corp. as Casodex (tradename); leuprorelin from Takeda Pharmaceutical Co., Ltd. as Leuplin (tradename); flutamide from Schering-Plough Corp. as Eulexin (tradename); fulvestrant from AstraZeneca Corp. as Faslodex (tradename); pegaptanib octasodium from Gilead Sciences, Inc. as Macugen (tradename); denileukin diftitox from Ligand Pharmaceuticals Inc. as Ontak (tradename); aldesleukin from Chiron Corp. as Proleukin (tradename); thyrotropin alfa from Genzyme Corp. as Thyrogen (tradename); arsenic trioxide from Cell Therapeutics, Inc. as Trisenox (tradename); bortezomib from Millennium Pharmaceuticals, Inc. as Velcade (tradename); capecitabine from Hoffmann-La Roche, Ltd. as Xeloda (tradename); and goserelin from AstraZeneca Corp. as Zoladex (tradename).

The term “antitumor agent” as used in the specification includes the above-described “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent”.

As used herein, the term “6-azaindole aminopyrimidine derivative” includes, but is not limited to, any compound having an aminopyrimidine analogue group which is substituted with an 6-azaindole derivative. It is exemplified by a compound of the above Formula I, and preferably any one compound of the below-mentioned (a) to (w): a compound which is:

(a) N-[4-(1-Isopropyl-1H-pyrrolo[2,3-c]pyridin-3-yl)-pyrimidin-2-yl]-benzamide (Example 1);

(b) [4-(1-Isopropyl-1H-pyrrolo[2,3-c]pyridin-3-yl)-pyrimidin-2-yl]-phenyl-amine (Example 3);

(c) 4-[4-(1-Isopropyl-1H-pyrrolo[2,3-c]pyridin-3-yl)-pyrimidin-2-ylamino]-benzenesulfonamide (Example 17);

(d) 3-[4-(1-Isopropyl-1H-pyrrolo[2,3-c]pyridin-3-yl)-pyrimidin-2-ylamino]-benzenesulfonamide (Example 59);

(e) 3-[4-(1-Isopropyl-1H-pyrrolo[2,3-c]pyridin-3-yl)-pyrimidin-2-ylamino]-4-methoxy-N-methyl-benzamide (Example 71);

(f) 3-Bromo-5-[4-(1-isopropyl-1H-pyrrolo[2,3-c]pyridine-3-yl)-pyrimidin-2-ylamino]-benzoic acid (Example 73);

(g) 5-[4-(1-Isopropyl-1H-pyrrolo[2,3-c]pyridin-3-yl)-pyrimidin-2-ylamino]-2-methyl-benzenesulfonamide (Example 80);

(h) N-(2-Hydroxy-ethyl)-4-[4-(1-isopropyl-1H-pyrrolo[2,3 -c]pyridin-3 -yl)-pyrimidin-2-ylamino]-benzamide (Example 83);

(i) 3-[4-(1-Isopropyl-1H-pyrrolo[2,3-c]pyridin-3-yl)-pyrimidin-2-ylamino]-4-methyl-benzamide (Example 85);

(j) 3-[4-(1-Isopropyl-1H-pyrrolo[2,3-c]pyridin-3-yl)-pyrimidin-2-ylamino]-N-methyl-benzamide (Example 111);

(k) 3-Bromo-5-[4-(1-isopropyl-1H-pyrrolo[2,3-c]pyridin-3-yl)-pyrimidin-2-ylamino]-N-methyl-benzamide (Example 113);

(l) 3-Bromo-5-[4-(1-isopropyl-1H-pyrrolo[2,3-c]pyridin-3-yl)-pyrimidin-2-ylamino]-benzamide (Example 88);

(m) 3-[4-(1-Isopropyl-1H-pyrrolo[2,3-c]pyridin-3-yl)-pyrimidin-2-ylamino]-5-trifluoromethyl-benzoic acid (Example 89);

(n) 3-Chloro-5-[4-(1-isopropyl-1H-pyrrolo[2,3-c]pyridin-3-yl)-pyrimidin-2-ylamino]-benzoic acid (Example 90);

(o) 3-[4-(1-Isopropyl-1H-pyrrolo[2,3-c]pyridine-3-yl)-pyrimidin-2-ylamino]-4-methoxy-benzamide (Example 41);

(p) 3-[4-(1-Isopropyl-1H-pyrrolo[2,3-c]pyridin-3-yl)-pyrimidin-2-ylamino]-5-methoxy-benzamide (Example 114);

(q)4-Benzyloxy-3-[4-(1-isopropyl-1H-pyrrolo[2,3-c]pyridin-3-yl)-pyrimidin-2-ylamino]-benzamide (Example 94);

(r) 3-[4-(1-Isopropyl-1H-pyrrolo[2,3-c]pyridin-3-yl)-pyrimidin-2-ylamino]-5-trifluoromethyl-benzamide (Example 122);

(s) 3-[4-(1-Isopropyl-1H-pyrrolo[2,3-c]pyridin-3-yl)-pyrimidin-2-ylamino]-N-methyl-5-trifluoromethyl-benzamide (Example 123);

(t) 3-[4-(1-Isopropyl-1H-pyrrolo[2,3-c]pyridin-3-yl)-pyrimidin-2-ylamino]-5-trifluoromethoxy-benzamide (Example 115);

(u) 3-Chloro-5-[4-(1-isopropyl-1H-pyrrolo[2,3-c]pyridin-3-yl)-pyrimidin-2-ylamino]-N-methyl-benzamide (Example 125);

(v) 3-[4-(1-Isopropyl-1H-pyrrolo[2,3-c]pyridin-3-yl)-pyrimidin-2-ylamino]-5-methyl-benzamide (Example 116); or

(w) 3-[4-(1-Isopropyl-1H-pyrrolo[2,3-c]pyridin-3-yl)-pyrimidin-2-ylamino]-4-methoxy-benzonitrile (Example 46),

or a pharmaceutically acceptable salt or ester thereof.

Description of the Process for Preparation of Compound of Formula I

The following Schemes A through D provide useful details for preparing a compound of Formula I according to the present invention. The requisite intermediates are in most cases commercially available, and/or can be prepared in accordance with literature procedures.

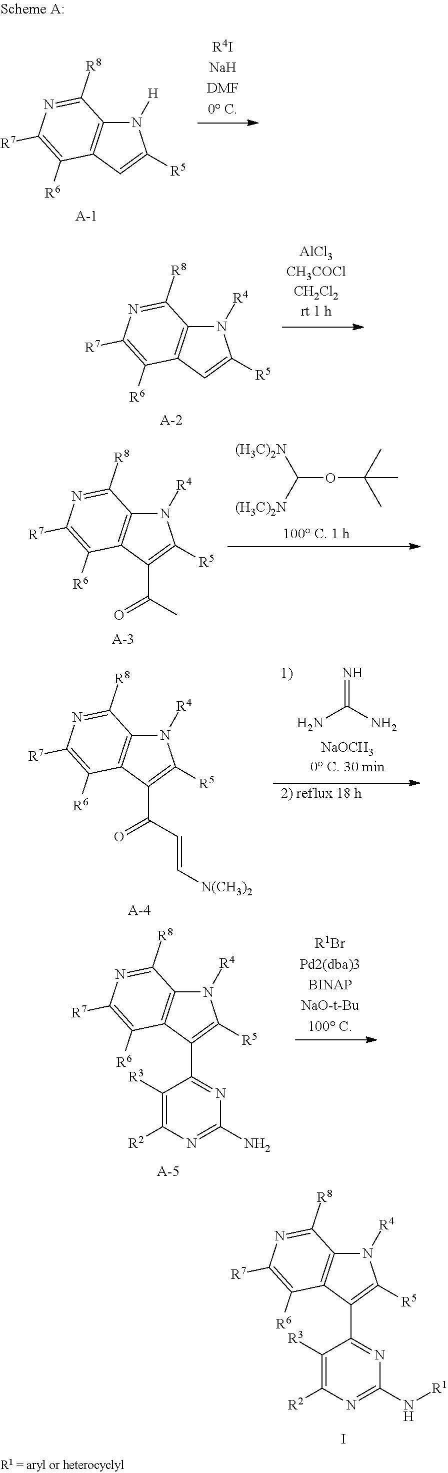

Scheme A illustrates the preparation of a compound of Formula I where R¹ is an aryl or heterocyclyl group. As shown in Scheme A, a suitably substituted 6-azaindole A-1 is reacted with an alkyl halide exemplified by R⁴-I to give the 6-azaindole substituted with R⁴ at the 1-position (A-2), which is then subjected to acetylation to afford the acetylated 6-azaindole A-3. Reaction of A-3 with Bredereck's reagent provides the 3-(3-Dimethylamino-1-oxo-2-propenyl)-6-azaindole derivative A-4, which is then reacted with guanidine, followed by reflux to afford the 6-azaindole aminopyrimidine derivative A-5. Following Buchwald-Hartwig reaction, a compound of Formula I according to the present invention can be obtained by reacting A-5 with an aryl or heterocyclyl bromide represented by R¹—Br in the presence of tris(dibenzylideneacetone)dipalladium, 2,2′-bis(diphenylphosphino)-1,1′-binapthyl, and sodium t-butoxide.

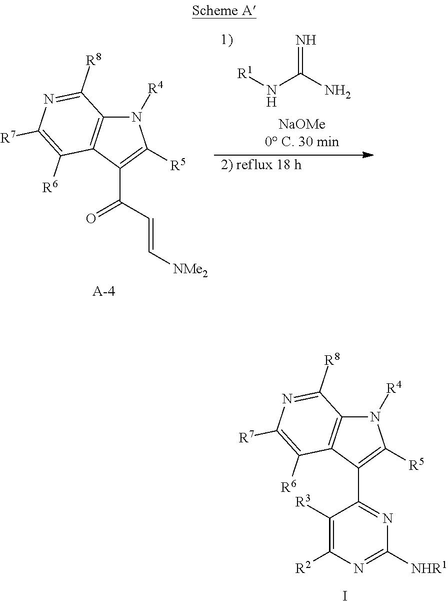

Alternatively, as shown in Scheme A′, A-4 is reacted with an aryl or heterocyclyl guanidine, followed by reflux, to give a compound of Formula I where R¹ is an aryl or heterocyclyl.

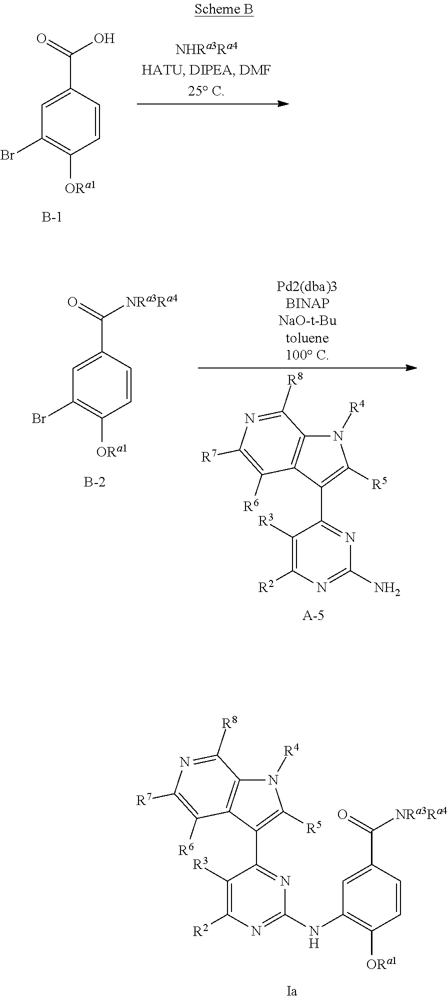

Scheme B illustrates the preparation of a compound of Formula I where R¹ is phenyl substituted with OR^{a1} and CONR^{a3}R^{a4} at the 2- and 5-positions, respectively; the compound is referred to as Formula Ia in Scheme B. As shown in Scheme B, a benzoic acid derivative B-1 is reacted with NHR^{3a}R^{a4} to give the corresponding benzamide derivative B-2. B-2 is then subject to a reaction with the 6-azaindole aminopyrimidine derivative A-5 in the presence of tris(dibenzylideneacetone)dipalladium, 2,2′-bis(diphenylphosphino)-1,1′-binapthyl, and sodium t-butoxide to afford the compound of Formula Ia.

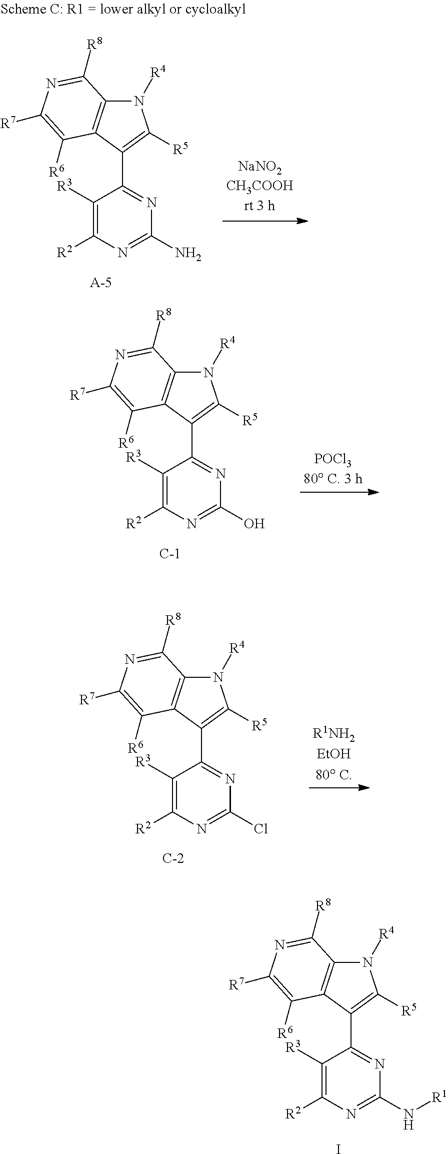

Scheme C illustrates the preparation of a compound of Formula I where R¹ is lower alkyl or cycloalkyl. As shown in Scheme C, A-5 is reacted with sodium nitrite to give the hydroxyl derivative C-1. C-1 is then subjected to chlorination to afford the chlorinated derivative C-2, which reacts with an alkyl amine represented by R¹NH₂, thereby giving the compound of Formula I.

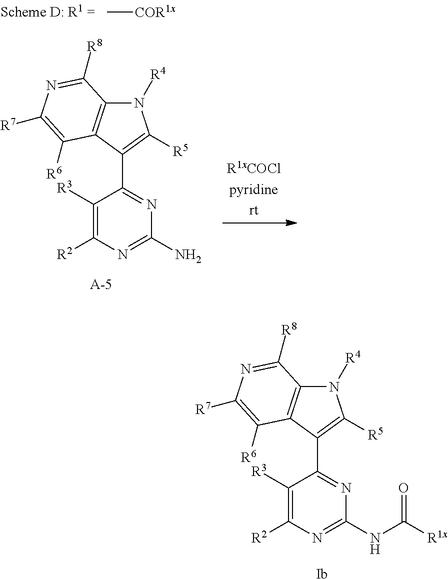

Scheme D illustrates the preparation of a compound of Formula I where R¹ is —COR^{1x}; the compound is referred to as Formula Ib in Scheme D. As shown in Scheme D, A-5 is reacted with an acyl chloride to afford the compound of Formula Ib.

Utility of Compounds of the Present Invention

The compounds of the invention are useful to inhibit the activity of NIK. In an embodiment, the NIK is human NIK. The compounds of the invention find use in a variety of applications. The compounds of the invention are used to treat or prevent cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like.

The compounds, compositions and methods provided herein are particularly deemed useful for the treatment and prevention of cancer including solid tumors such as skin, breast, brain, cervical carcinomas, testicular carcinomas, etc. In an embodiment, the present compounds are useful for treating cancer. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In an embodiment of the invention, cancers that may be treated by the compounds, compositions and methods of the invention include, in addition to the cancers listed above: Lung: bronchogenic carcinoma (non-small cell lung); Gastrointestinal: rectal, colorectal and colon; Genitourinary tract: kidney (papillary renal cell carcinoma); and Skin: head and neck squamous cell carcinoma.

In another embodiment, the compounds of the present invention are useful for treating or preventing cancer selected from: head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In yet another embodiment, the compounds of the present invention are useful for treating or preventing cancer selected from: histiocytic lymphoma, lung adenocarcinoma, small cell lung cancer, pancreatic cancer, liver cancer, gastric cancer, colon cancer, multiple myeloma, glioblastomas and breast carcinoma. In still another embodiment, the compounds of the instant invention are useful for treating cancer selected from: histiocytic lymphoma, lung adenocarcinoma, small cell lung cancers, pancreatic cancer, liver cancer, gastric cancer, colon cancer, multiple myeloma, glioblastomas and breast carcinoma.

In another embodiment, the compounds of the present invention are useful for the prevention or modulation of the metastases of cancer cells and cancer. In particular, the compounds of the instant invention are useful to prevent or modulate the metastases of ovarian cancer, childhood hepatocellular carcinoma, metastatic head and neck squamous cell carcinomas, gastric cancers, breast cancer, colorectal cancer, cervical cancer, lung cancer, nasopharyngeal cancer, pancreatic cancer, glioblastoma and sarcomas.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

Biological assays using compounds of the present invention

I. NIK Inhibitory Activity

(1) Purification of NIK Enzyme

Truncated human NIK [319-947(end) amino acids of accession number AAH35576] was expressed in sf9 cells as N-terminus gluthatione-S-transferase (GST)-tagged protein using baculovirus expression system. The sf9 cells were harvested and lysed, and then the GST-tagged human NIK protein was applied onto a glutathione column (GSTrap, GE Healthcare) and eluted from the column with reduced glutathione. The active fractions were desalted with a desalting column (PD-10, GE Healthcare) to give a purified enzyme.

(2) Synthesis of SRPKtide

The synthesis of the SRPKtide (Arg-Ser-Arg-Ser-Arg-Ser-Arg-Ser-Arg-Ser-Arg-Ser-Arg-Ser-Arg-NH₂) (SEQ.ID.NO.:1) was carried out by the Fmoc/tBu strategy on Pioneer™ peptide synthesizer (Applied Biosystems, Foster City, Calif.), using HATU/Diisopropylethylamine as an activating reagent. NovaSyn° TGR resin (Novabiochem, Laufelfingen, Switzerland) was used as a solid support, and Fmoc-Arg(Pbf) and Fmoc-Ser(tBu) was used as a protected amino acid.

Specifically, the solid phase synthesis of SRPKtide was carried out with 0.2 mmol of NovaSyn® TGR resin using a peptide synthesizer. The synthesized resin was deprotected and cleaved with 15 ml of TFA/thioanisole/ethandithiol/m-cresol (95/2.5/1.5/1) at room temperature for 1.5 hours. Then the resin was filtered off and the cold diethyl ester was added to solidify the peptide. The precipitate was collected by centrifugation, and 438.7 mg of the crude peptide was obtained. This crude peptide was purified with preparative reverse-phase HPLC at a flow rate of 10 ml/min using a linear gradient from 100% Solvent A1:0% Solvent B1 to 70% Solvent A1:30% Solvent B1 over 20 min (retention time; 14.2 min), and the fraction containing the target peptide was collected. After lyophilization, 102.2 mg of SRPKtide was obtained. The characterization of the obtained SRPKtide was performed by LC/MS at a flow rate of 0.2 ml/min using a liner gradient from 100% Solvent A2:0% Solvent B2 to 70% Solvent A2:30% Solvent B2 over 20 min (retention time; 13.43 min)

Analytical conditions for LC/MS and preparative reversed-phase HPLC are as follows:
Purification of the peptide: Preparative reversed-phase HPLC (Waters 600 system, waters, Milford, Mass.) on YMC-Pack ODS-AQ column (20×250 mm; YMC, Kyoto, Japan); the mobile phase consisted of 0.1% TFA in water (Solvent A1) and 0.1% TFA in acetonitrile (Solvent B1).
Characterization of the peptide: LC/MS (Waters Platform ZMD2000, Waters alliance 2690, Waters 996, Waters, Milford, Mass.) on YMC-Pack ODS-AQ column (2.0×150 mm; YMC, Kyoto, Japan); the mobile phase consisted of 0.1% TFA in water (Solvent A2) and 0.1% TFA in acetonitrile (Solvent B2).
HATU: O-(7-Azabenzotriazol-1-yl)-N,N,N′,N′,tetramethyluronium hexafluorophosphate
Pbf: 2,2,4,6,7-pentamethyldihydrobenzofuran-5-sulfonyl

(3) Measurement of Activity of NIK

For measurement of the activity of NIK, the SRPKtide peptide (Arg-Ser-Arg-Ser-Arg-Ser-Arg-Ser-Arg-Ser-Arg-Ser-Arg-Ser-Arg-NH₂) (SEQ.ID.NO.:1) was synthesized as a substrate, as described above.

The phosphorylation reaction was conducted using 384 well plate, and the reaction volume was 10.5 μL/well. The reaction buffer is comprised of 10 mM 3-Morpholinopropanesulfonic acid buffer (pH 7.4), 5 mM magnesium chloride, 1 mM O,O′-Bis (2-aminoethyl) ethyleneglycol-N,N,N′,N′-tetraacetic acid (EGTA) and 1 mM dithiothreitol. Thereto, the purified NIK protein, 20 μM of the peptide substrate, 10 μM of adenosine 5′-triphosphate (ATP) and 0.5 μCi [γ-33P] ATP solution were added, and then the reaction was carried out at 25° C. for 240 minutes. The [γ-33P]-labeled ATP was purchased from PerkinElmer Inc.

After the termination of the reaction by adding 20 μL of 350 mM phosphoric acid (H3PO4), the substrate peptide was adsorbed on a filter plate (Millipore Multiscreen, MZPHN0W50). The substrate-bound filter plate was washed with 130 mM phosphoric acid for several times and then 8 μL of Microscinti-O (PerkinElmer Inc.) was added to the each well. The radiation activity of the peptide was measured with TopCount NXT Microscintillation Counter (PerkinElmer Inc.).

The compound to be tested was diluted in dimethylsulfoxide (DMSO) and then 0.5 μL of this solution was added to each well. Each control well was provided by adding 0.5 μL of DMSO to the well in place of the DMSO solution containing the compound to be tested.

TABLE 1

| | Inhibitory |
|---|---|
| | activity for NIK |
| | (IC₅₀, nM) |
| Example 3 | 422.9 |
| Example 71 | 26.08 |
| Example 85 | 63.37 |
| Example 94 | 84.47 |

II. Method for Deternimation of the Cellular NIK Inhibitory Activities Using ELISA Based IKKalpha Phosphorylation Assay

The cellular NIK inhibitory activity of the compound of the Formula I according to the invention will be explained below.

(1) Reagents and Cell Lines

Dulbecco's Modified Eagle Medium (DMEM, high glucose), penicillin/streptomycin solution, and hygromycin solution were purchased from Invitrogen Corp. Tet system approved fetal bovine serum (FBS) and puromycin was purchased from Clontech Laboratories, Inc.

NF-kappa B reporter genes (Clontech Laboratories, Inc.) was introduced into U-2 OS Tet-On cell line (Clontech Laboratories, Inc.) by the co-transfection with puromycin resistance gene (Clontech Laboratories, Inc.). Then human NIK cDNA was subcloned into pTRE2hyg vector (Clontech Laboratories, Inc.), and this plasmid was transfected into the established U-2 OS Tet-On cells possessing NF-kappa B reporter genes. NIK protein expression in this established cells (U-2 OS Tet-On-NIK) was controlled in a doxycycline dependent manner. The cells were cultured in DMEM containing 10% FBS, 100 units/ml of penicillin, 0.1 mg of streptomycin sulfate, 100 microgram/ml of hygromycin and 1 microgram/ml of puromycin (Growth medium).

(2) Determination of Cellular NIK Inhibitory Activity Using ELISA-Based IKKalpha Phosphorylation Assay

U-2 OS Tet-On-NIK cells were plated on 96-well plate (Nunc) at 20000 cells per well and were incubated overnight at 37° C. in 5% CO₂-95% air. The tested compound was diluted in dimethylsulfoxide (DMSO) and further diluted with a growth medium. Then the compound solution and doxycycline (Clontech Laboratories, Inc.) solution (final working concentration is 2 micrograms/ml) were simultaneously added to the each well. The cells were incubated for further 24 hours at 37° C. in 5% CO₂-95% air, and were lysed by incubation with 50 mM Tris-hydrochloride buffer (pH 7.4) containing 150 mM sodium chloride, 1% NP-40, protease inhibitor cocktail (Sigma-Aldrich, Inc.) and phosphatase inhibitor cocktail (Pierce Biotechnology, Inc.) at 4° C. for 2 hours. Cell lysates were stored at −80° C. until use.

Buffers used in ELISA-based IKKalpha phosphorylation assay are listed below.
50 mM Carbonate-Bicarbonate buffer (Sigma-Aldrich, Inc.): For use in coating ELISA plate with antibody
Dulbecco's phosphate-buffer saline (Invitrogen Corp.): D-PBS
50 mM Tris-hydrochloride buffer (pH 8.0) containing 150 mM sodium chloride and 0.05% Tween-20: TBS-T
TBS-T containing 1% bovine serum albumin (BSA): For use in antibody dilution
TBS-T containing 0.1% bovine serum albumin (BSA): For use in wash out

Rabbit anti-IKKalpha [pSpS176/180] phosphospecific antibody (Biosource International, Inc.) or rabbit anti-IKKalpha antibody (Abcam, Inc.) was incubated on wells of an MAXISORP ELISA plate (Nunc) at 4° C. for overnight. After washing out the antibodies, the wells were blocked by blocking agents (5% BSA in D-PBS) for 2 hours at room temperature. Then cell lysates in TBS-T were added to each well and were incubated at 4° C. for overnight. After removal of the lysates and wash out, the bound IKKalpha proteins were proved with mouse anti-IKKalpha antibody (BD biosciences) and detected by horseradish peroxidase (HRP)-conjugated horse anti-mouse IgG (H+L) antibody (Cell Signaling Technology, Inc.). After stopping HRP-reactions (SureBlue Reserve™ TMB substrate, Kirkegaard & Perry Laboratories, Inc.) by 1N hydrochloric acid, the optical density in each well was determined using a SpectraMax Plus 384 (Molecular Devices Corporation) at 450 nm.

Cellular NIK inhibitory activity of the test compound was represented as IC₅₀ value determined by the following procedure.

Optical density of phospho-IKKalpha in each sample was normalized using the ratio of phospho-IKKalpha to total IKKalpha. The % of Control value was calculated by the following equation.

% of Control=(S [NIK+]−C [NIK−])/(C [NIK+]−C [NIK−])×100
Normalized phospho-IKKalpha in control sample with NIK induction: C [NIK+]
Normalized phospho-IKKalpha in control sample without NIK induction: C [NIK−]
Normalized phospho-IKKalpha in compound-treated sample with NIK induction: S [NIK+]

Then the IC₅₀ value for NIK dependent IKKalpha phosphorylation was determined.

TABLE 2

| | Cellular NIK |
|---|---|
| | inhibitory activity |
| | (IC₅₀, nM) |
| Example 71 | 200 |
| Example 85 | 140 |
| Example 94 | 230 |

As shown in Table 2, the compound according to the invention exhibited potent cellular NIK inhibitory activities in U-2 OS Tet-On-NIK cells.

III. ROCK2 Inhibitory Activity

(1) Purification of ROCK2 Enzyme

cDNA of N-terminal His-tagged human ROCK2 catalytic domain [11-552 amino acids of accession number NP_{—}00004841.2] was integrated into an expression vector, which was then highly expressed in sf9 cells. The sf9 cells were harvested and lysed, and then the His-tagged human ROCK2 protein was applied onto HisTrap HP (GE Healthcare) and eluted from the column with imidazole. The active fractions were desalted with a desalting column (PD-10, GE Healthcare) to give a purified enzyme.

(2) Measurement of Activity of ROCK2

For measurement of the activity of ROCK2, the substrate used was a synthetic peptide (5-FAM (5-carboxyfluorescein)-Ala-Lys-Arg-Arg-Arg-Leu-Ser-Ser-Leu-Arg-Ala-OH) (SEQ.ID.NO.:2) (Trauger J W. et. al, Biochemistry, 41, 8948-8953 (2002)), which was custom-made by JPT Peptide Technologies GmbH.

For detection of phosphorylation of the substrate, IMAP® technology (Molecular Devices, Co. Ltd.) (Gaudet E W. et. al, J. Biomol. Screen, 8, 164-175(2003)) was used.

The phosphorylation reaction was conducted using 384 well plate, and the reaction volume was 10.5 μL/well. The reaction buffer is comprised of 50 mM Tris-chloride buffer (pH 7.5), 10 mM magnesium acetate, 1 mM O,O′-Bis(2-aminoethyl)ethyleneglycol-N,N,N′,N′-tetraacetic acid (EGTA) and 1 mM dithiothreitol. Thereto, the purified ROCK2 protein, 100 nM of the peptide substrate, and 5 μM of adenosine 5′-triphosphate were added, and then the reaction was carried out at 30° C. for 150 minutes.

Thereafter, in order to terminate and detect the reaction, 30 μL of the IMAP (registered trademark) binding reagent (IMAP Progressive Binding Reagent) that had been diluted (1:500) in the 1× IMAP binding buffer A (IMAP Progressive Binding Buffer A, 5× stock) was added to each well. The solution stood still for 30 minutes in the dark, and then fluorescence polarization was measured using a high-end microplate reader (excitation wavelength: 485 nm; emission wavelength: 520 nm).

The compound to be tested was diluted in dimethylsulfoxide (DMSO) and then 0.5 μL of this solution was added to each well. Each control well was provided by adding 0.5 μL of DMSO to the well in place of the DMSO solution containing the compound to be tested. In Table 3, the selectivity of NIK over ROCK2 was calculated by dividing the value of IC₅₀ for ROCK2 by the value of IC₅₀ for NIK.

TABLE 3

| | Inhibitory | |
|---|---|---|
| | activity for | Selectivity of |
| | ROCK2 (IC₅₀, | NIK over |
| | nM) | ROCK2 |
| Example 3 | 86.04 | 0.20 |
| Example 71 | 4520 | 173.3 |
| Example 85 | 2589 | 40.9 |
| Example 94 | 1006 | 11.9 |

The compound of the present invention is useful as an antitumor/anti-cancer/anti-inflammatory agent since it exhibits good NIK inhibitory activity. Thus, it is considered that a pharmaceutical composition or NIK inhibitor containing the 6-azaindole aminopyrimidine derivative according to the invention or a pharmaceutically acceptable salt or ester thereof, or an antitumor/anti-cancer agent containing the compound according to the invention or a pharmaceutically acceptable salt or ester thereof, is effective in the treatment of cancer patients. Also, as indicated above, in the case where R¹ is phenyl disubstituted at the 2- and 5-positions with C₁₋₆ alkyl or —OR^{a1} and with —CONR^{a3}R^{a4}, respectively, the compounds of the present invention exhibited excellent NIK selective inhibitory activity as compared with inhibitory activity against other kinases such as ROCK2.

The above-mentioned pharmaceutical composition and inhibitor, and the above-mentioned antitumor agent may contain a pharmaceutically acceptable carrier or diluent. As used herein, the “pharmaceutically acceptable carrier or diluent” refers to excipients [e.g., fats, beeswax, semi-solid and liquid polyols, natural or hydrogenated oils, etc.]; water (e.g., distilled water, particularly distilled water for injection, etc.), physiological saline, alcohol (e.g., ethanol), glycerol, polyols, aqueous glucose solution, mannitol, plant oils, etc.); additives [e.g., extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant], and the like.

Next, the above-described “pharmaceutically acceptable salt or ester” will be explained below: when the compound according to the invention is used as an antitumor/anti-cancer agent or the like, it may be also used in a form of pharmaceutically acceptable salt. Typical examples of the pharmaceutically acceptable salt include a salt with an alkali metal such as sodium and potassium; a salt with an inorganic acid, such as hydrochloride, sulfate, nitrate, phosphate, carbonate, hydrogen carbonate, and perchlorate; a salt with an organic acid, such as acetate, propionate, lactate, maleate, fumarate, tartrate, malate, citrate, and ascorbate; a salt with sulfonic acid, such as methanesulfonate, isethionate, benzenesulfonate, and toluenesulfonate; a salt with acidic amino acid, such as aspartate and glutamate; and the like. A pharmaceutically acceptable salt of the Compound represented by the Formula I is preferably a salt with an inorganic acid, such as hydrochloride, sulfate, nitrate, phosphate, carbonate, hydrogen carbonate, and perchlorate.

A method for preparing a pharmaceutically acceptable salt of the compound according to the invention may be carried out by an appropriate combination of those methods that are conventionally used in the field of organic synthetic chemistry. A specific example thereof is a method in which a solution of the compound according to the invention in its free form is subjected to neutralization titration with an alkaline solution or an acidic solution.

Examples of the ester of the compound according to the invention include methyl ester and ethyl ester. Such esters can be prepared by esterification of a free carboxyl group according to a conventional method.

With regard to each preparation according to the invention, various preparation forms can be selected, and examples thereof include oral preparations such as tablets, capsules, powders, granules or liquids, or sterilized liquid parenteral preparations such as solutions or suspensions, suppositories, ointments and the like.

Solid preparations can be prepared in the forms of tablet, capsule, granule and powder without any additives, or prepared using appropriate carriers (additives). Examples of such carriers (additives) may include saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

These solid preparations such as tablets, capsules, granules and powders may generally contain, for example, 0.1 to 100% by weight, and preferably 5 to 98% by weight, of the compound of the Formula I as an active ingredient, based on the total weight of the preparation.

Liquid preparations are produced in the forms of suspension, syrup, injection and drip infusion (intravenous fluid) using appropriate additives that are conventionally used in liquid preparations, such as water, alcohol or a plant-derived oil such as soybean oil, peanut oil and sesame oil.

In particular, when the preparation is administered parenterally in a form of intramuscular injection, intravenous injection or subcutaneous injection, appropriate solvent or diluent may be exemplified by distilled water for injection, an aqueous solution of lidocaine hydrochloride (for intramuscular injection), physiological saline, aqueous glucose solution, ethanol, polyethylene glycol, propylene glycol, liquid for intravenous injection (e.g., an aqueous solution of citric acid, sodium citrate and the like) or an electrolytic solution (for intravenous drip infusion and intravenous injection), or a mixed solution thereof.

Such injection may be in a form of a preliminarily dissolved solution, or in a form of powder per se or powder associated with a suitable carrier (additive) which is dissolved at the time of use. The injection liquid may contain, for example, 0.1 to 10% by weight of an active ingredient based on the total weight of the preparation.

Liquid preparations such as suspension or syrup for oral administration may contain, for example, 0.1 to 10% by weight of an active ingredient based on the total weight of the preparation.

Each preparation according to the invention can be prepared by a person having ordinary skill in the art according to conventional methods or common techniques. For example, a preparation containing another antitumor agent that is used in combination with the compound represented by the above Formula I, can be prepared, if the preparation is an oral preparation, for example, by mixing an appropriate amount of the antitumor agent with an appropriate amount of lactose and filling this mixture into hard gelatin capsules which are suitable for oral administration. On the other hand, preparation can be carried out, if the preparation containing the antitumor agent is an injection, for example, by mixing an appropriate amount of the antitumor agent with an appropriate amount of 0.9% physiological saline and filling this mixture in vials for injection.

In a method of treatment according to the invention, preferred therapeutic unit may vary in accordance with, for example, the administration route of the compound represented by the Formula I, the type of the compound represented by the Formula I used, and the dosage form of the compound represented by the Formula I used; the type, administration route and dosage form of the other antitumor agent used in combination; and the type of cells to be treated, the condition of patient, and the like. The optimal treatment under the given conditions can be determined by a person skilled in the art, based on the set conventional therapeutic unit and/or based on the content of the present specification.

In a method of treatment according to the invention, the therapeutic unit for the compound represented by the above Formula I may vary in accordance with, specifically, the type of compound used, the type of compounded composition, application frequency and the specific site to be treated, seriousness of the disease, age of the patient, doctor's diagnosis, the type of cancer, or the like. However, as an exemplary reference, the daily dose for an adult may be within a range of, for example, 1 to 1,000 mg in the case of oral administration. In the case of parenteral administration, the daily dose may be within a range of, for example, 1 to 100 mg/m² (body surface area). Here, in the case of intravenous drip infusion, administration may be continuously carried out for, for example, 1 to 48 hours. Moreover, the administration frequency may vary depending on the administering method and symptoms, but it is, for example, once to five times a day. Alternatively, periodically intermittent administration such as administration every other day, administration every two days or the like may be employed as well in the administering method. The period of withdraw from medication in the case of parenteral administration is, for example, 1 to 6 weeks.

Although the therapeutic unit for the other antitumor agent used in combination with the compound represented by the Formula I is not particularly limited, it can be determined, if needed, by those skilled in the art according to known literature. Examples may be as follows.

The therapeutic unit of 5-fluorouracil (5-FU) is such that, in the case of oral administration, for example, 200 to 300 mg per day is administered in once to three times consecutively, and in the case of injection, for example, 5 to 15 mg/kg per day is administered once a day for the first 5 consecutive days by intravenous injection or intravenous drip infusion, and then 5 to 7.5 mg/kg is administered once a day every other day by intravenous injection or intravenous drip infusion (the dose may be appropriately increased or decreased).

The therapeutic unit of S-1 (Tegafur, Gimestat and Ostat potassium) is such that, for example, the initial dose (singe dose) is set to the following standard amount in accordance with the body surface area, and it is orally administered twice a day, after breakfast and after dinner, for 28 consecutive days, followed by withdrawal from medication for 14 days. This is set as one course of administration, which is repeated. The initial standard amount per unit body surface area (Tegafur equivalent) is 40 mg in one administration for an area less than 1.25 m²; 50 mg in one administration for an area of 1.25 m² to less than 1.5 m²; 60 mg in one administration for an area of 1.5 m² or more. This dose is appropriately increased or decreased depending on the condition of the patient.

The therapeutic unit for gemcitabine is, for example, 1 g as gemcitabine/m² in one administration, which is administered by intravenous drip infusion over a period of 30 minutes, and one administration per week is continued for 3 weeks, followed by withdrawal from medication on the fourth week. This is set as one course of administration, which is repeated. The dose is appropriately decreased in accordance with age, symptom or development of side-effects.

The therapeutic unit for doxorubicin (e.g., doxorubicin hydrochloride) is such that, for example, in the case of intravenous injection, 10 mg (0.2 mg/kg) (titer) is administered once a day by intravenous one-shot administration for 4 to 6 consecutive days, followed by withdrawal from medication for 7 to 10 days. This is set as one course of administration, which is repeated two or three times. Here, the total dose is preferably 500 mg (titer)/m² (body surface area) or less, and it may be appropriately increased or decreased within the range.

The therapeutic unit for etoposide is such that, for example, in the case of intravenous injection, 60 to 100 mg/m² (body surface area) per day is administered for 5 consecutive days, followed by withdrawal from medication for three weeks (the dose may be appropriately increased or decreased). This is set as one course of administration, which is repeated. Meanwhile, in the case of oral administration, for example, 175 to 200 mg per day is administered for 5 consecutive days, followed by withdrawal from medication for three weeks (the dose may be appropriately increased or decreased). This is set as one course of administration, which is repeated.

The therapeutic unit for docetaxel (docetaxel hydrate) is such that, for example, 60 mg as docetaxel/m² (body surface area) is administered once a day by intravenous drip infusion over a period of 1 hour or longer at an interval of 3 to 4 weeks (the dose may be appropriately increased or decreased).

The therapeutic unit of paclitaxel is such that, for example, 210 mg/m² (body surface area) is administered once a day by intravenous drip infusion over a period of 3 hours, followed by withdrawal from medication for at least 3 weeks. This is set as one course of administration, which is repeated. The dose may be appropriately increased or decreased.

The therapeutic unit for cisplatin is such that, for example, in the case of intravenous injection, 50 to 70 mg/m² (body surface area) is administered once a day, followed by withdrawal from medication for 3 weeks or longer (the dose may be appropriately increased or decreased). This is set as one course of administration, which is repeated.

The therapeutic unit for carboplatin is such that, for example, 300 to 400 mg/m² is administered once a day by intravenous drip infusion over a period of 30 minutes or longer, followed by withdrawal from medication for at least 4 weeks (the dose may be appropriately increased or decreased). This is set as one course of administration, which is repeated.

The therapeutic unit for oxaliplatin is such that 85 mg/m² is administered once a day by intravenous injection, followed by withdrawal from medication for two weeks. This is set as one course of administration, which is repeated.

The therapeutic unit for irinotecan (e.g., irinotecan hydrochloride) is such that, for example, 100 mg/m² is administered once a day by intravenous drip infusion for 3 or 4 times at an interval of one week, followed by withdrawal from medication for at least two weeks.

The therapeutic unit for topotecan is such that, for example, 1.5 mg/m² is administered once a day by intravenous drip infusion for 5 days, followed by withdrawal from medication for at least 3 weeks.

The therapeutic unit for cyclophosphamide is such that, for example, in the case of intravenous injection, 100 mg is administered once a day by intravenous injection for consecutive days. If the patient can tolerate, the daily dose may be increased to 200 mg. The total dose is 3,000 to 8,000 mg, which may be appropriately increased or decreased. If necessary, it may be injected or infused intramuscularly, intrathoracically or intratumorally. On the other hand, in the case of oral administration, for example, 100 to 200 mg is administered a day.

The therapeutic unit for gefitinib is such that 250 mg is orally administered once a day.

The therapeutic unit for cetuximab is such that, for example, 400 mg/m² is administered on the first day by intravenous drip infusion, and then 250 mg/m² is administered every week by intravenous drip infusion.

The therapeutic unit for bevacizumab is such that, for example, 3 mg/kg is administered every week by intravenous drip infusion.

The therapeutic unit for trastuzumab is such that, for example, typically for an adult, once a day, 4 mg as trastuzumab/kg (body weight) is administered initially, followed by intravenous drip infusion of 2 mg/kg over a period of 90 minutes or longer every week from the second administration.

The therapeutic unit for exemestane is such that, for example, typically for an adult, 25 mg is orally administered once a day after meal.

The therapeutic unit for leuprorelin (e.g., leuprorelin acetate) is such that, for example, typically for an adult, 11.25 mg is subcutaneously administered once in 12 weeks.

The therapeutic unit for imatinib is such that, for example, typically for an adult in the chronic phase of chronic myelogenous leukemia, 400 mg is orally administered once a day after meal.

The therapeutic unit for a combination of 5-FU and leucovorin is such that, for example, 425 mg/m² of 5-FU and 200 mg/m² of leucovorin are administered from the first day to the fifth day by intravenous drip infusion, and this course is repeated at an interval of 4 weeks.

The therapeutic unit for sorafenib is such that, for example, 200 mg is orally administered twice a day (400 mg per day) at least 1 hour before or 2 hours after eating.

The therapeutic unit for sunitinib is such that, for example, 50 mg is orally administered once a day for four weeks, followed by 2 weeks off

### EXAMPLES

The following preparations illustrate methods for the preparation of compounds according to the present invention, as well as those for the preparation of intermediates. It should be evident to those skilled in the art that appropriate substitution of both the materials and methods disclosed herein will produce the examples illustrated below and those encompassed by the scope of the invention.

Before describing each preparation of intermediates and compounds according to the present invention, the following should be noted in general:

All temperatures are given in degrees Celsius. Reagents were purchased from commercial sources or prepared in accordance with literature procedures.

Unless otherwise noted, HPLC purification was performed by redissolving a residue in a small volume of CH₃OH or other appropriate solvent. The solution was then purified via preparatory reverse-phase purification system using a Varian Dynamax HPLC 21.4 mm Microsorb Guard-8 C₁₈ column. In general, Solvent A was a mixture of 5% CH₃CN:95% H₂O:0.1% CF₃COOH while Solvent B was a mixture of 95% CH₃CN:5% H₂O:0.1% CF₃COOH. Details are as follows: A typical run would be from 0% Solvent B:100% Solvent A to 100% Solvent B:0% Solvent A over a period of 5 minutes, followed by a hold at 100% Solvent B, before it was re-equilibrated back to the initial starting gradient. Total run time was 7 minutes. The resulting fractions were analyzed, combined as appropriate, and then evaporated to provide purified material. Unless otherwise noted, all compounds resulting from the reverse-phase HPLC purification were characterized as the corresponding TFA salts.

Proton magnetic resonance (¹H NMR) spectra were recorded on either a Varian NOVA 400 MHz (¹H) NMR spectrometer, or Varian INOVA 500 MHz (¹H) NMR spectrometer. All spectra were determined in the solvents indicated below. Although chemical shifts are reported in parts per million (ppm) downfield of tetramethylsilane, they are referenced to the residual proton peak of the respective solvent peak for ¹H NMR. Interproton coupling constants are reported in Hertz (Hz). LCMS spectra were obtained using a ThermoFinnigan AQA MS ESI instrument. The samples were sent through a Phenomenex Aqua 5 micron C_{B} 125 Å 50×4.60 mm column. For purity analysis, Solvent C was H₂O with 0.1% formic acid, and Solvent D was CH₃OH with 0.1% formic acid. For purity analysis of the freebase intermediates a gradient of 45% D:C to 95% D:C over 5 minutes and then a 3-minute hold at 95% D:C was used. For purity analysis of the trifluoroacetate salts, a gradient of 5% D:C to 95% D:C over 5 minutes and then a 1 minute hold at 95% D:C was used. The spray setting for the MS probe was at 350 μL/min with a cone voltage at 3 kV and a probe temperature at 450° C.

Abbreviations used herein are: HPLC (high-performance liquid chromatography); TFA (trifluoroacetic acid); ¹H NMR (proton nuclear magnetic resonance); LCMS (liquid chromatograph-mass spectrometer); MS (mass spectrometer); THF (tetrahydrofuran); eq (equivalents); NH₄Cl (ammonium chloride); DMF (N,N-dimethylformamide); NaCl (sodium chloride) 1NaOMe (sodium methoxide); EtOH (ethanol); Pd/C (palladium on carbon); Et₃N (triethylamine); AcOH (acetic acid); DMSO (dimethyl sulfoxide); DCM (dichloromethane); EtOAc (ethyl acetate); LC/MS (liquid chromatogram-mass spectrometer); BINAP (2,2′-bis(diphenylphosphino)-1,1′-binapthyl); NaO-t-Bu (sodium t-butoxide); Pd₂(dba)₃ (tris(dibenzylideneacetone)dipalladium); BnCH₂Br (benzyl bromide); rt (room temperature); n-(normal); ESI (electrospray ionization) x-phos, xantphos (4,5-bis(diphenylphosphino)-9,9-dimethylxanthene); DIAD (diisopropyl azodicarboxylate); NBS (N-bromosuccinimide); EDCI(1-ethyl-3-(3-dimethylaminopropyl) carbodiimide); dppf (1,1′-bis(diphenylphosphanyl)ferrocene); HATU(o-(7-azabenzotriazol-1-yl)-N,N,N′,N′-tetramethyluronium hexafluorophosphate); DIPEA (diisopropylethylamine); Tf2O (trifluoromethanesulfonic anhydride); NIS (N-iodosuccinimide); TsCl (p-toluenesulfonyl chloride); TBAF (tetrabutylammonium fluoride); DMAP (dimethylaminopyridine); ACN (acetonitrile); DMA (dimethylacetoamide); MCPBA (m-chloroperbenzoic acid); TBAB (tetrabutylammonium bromide); DIBAL-H (diisobutylaluminium hydride); NCS (N-chlorosuccinimide); dppe (1,2-Bis(diphenylphosphino)ethane); HOBT (1-hydroxybenzotriazole); 18-crown-6 (1,4,7,10,13,16-hexaoxacyclooctadecane).

Schemes E, F, and G illustrate the preparation of Intermediate E-7.

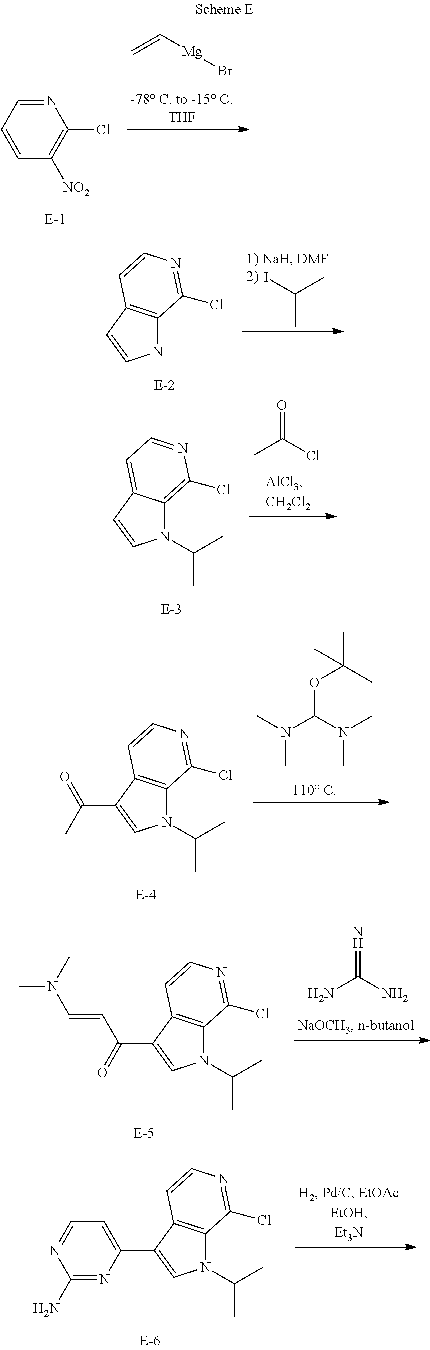

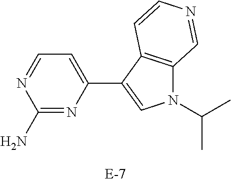

### Preparation of 7-Chloro-1H-pyrrolo[2,3-c]pyridine (E-2)

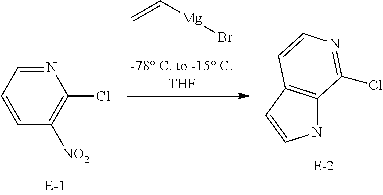

To a solution of 2-chloro-3-nitropyridine E-1 (20 g, 126 mmol, 1 eq.) in anhydrous THF (800 mL) cooled to −78° C. was added a solution of vinyl magnesium bromide (400 mL of a 1.0 M solution in THF, 400 mmol, 3.2 eq.) by cannula. The mixture was stirred for 6 hours while allowing the mixture to slowly warm to −15° C. After this period, a solution of saturated aqueous NH₄Cl was added, followed by the addition of H₂O, and the mixture was extracted with ethyl acetate three times. The combined organic phases were concentrated onto Celite (trade name) and the material was subjected to silica gel chromatography ({CHCl₃ (90%), CH₃OH (10%), concentrated aqueous NH₄OH (1%)}:CHCl₃) to provide Compound E-2: LCMS m/e 153 (M+H); ¹H NMR (400 MHz, Methanol-d₄) δppm 6.64 (d, J=3.03 Hz, 1H), 7.56 (d, J=5.56 Hz, 1H), 7.58 (d, J=3.07 Hz, 1H), 7.86 (d, J=5.56 Hz, 1H).

### Preparation of 7-Chloro-1-isopropyl-1H-pyrrolo[2,3-c]pyridine (E-3)

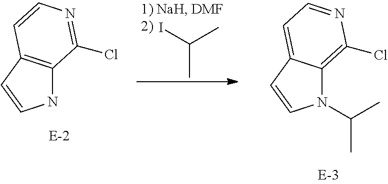

To a 0° C. solution of Compound E-2 (13.98 g, 91.6 mmol, 1 eq.) in DMF (700 mL) was added NaH (5.5 g of a 60% dispersion in mineral oil, 229.2 mmol, 2.5 eq.) in portions. The mixture was allowed to stir at room temperature for 1 hour, then 2-iodopropane (18.3 mL, 183.3 mmol, 2.0 eq.) was added and the resulting mixture was allowed to stir at room temperature for 6 hours. The mixture was then charged with an additional amount of NaH (5 g of a 60% dispersion in mineral oil, 208 mmol, 2.3 eq.) and 2-iodopropane (9 mL, 92 mmol, 1 eq.) and was allowed to stir at room temperature. After 16 hours, the mixture was cooled to 0° C. and MeOH was added. Next, H₂O and ethyl acetate were added and the mixture was stirred. Stirring was stopped and the layers became separated. The aqueous phase was extracted twice with ethyl acetate. The combined organic phases were washed with H₂O twice, followed by saturated aqueous NaCl solution, dried over Na₂SO₄, and concentrated by rotary evaporation. The residue was purified by silica gel chromatography using ethyl acetate and heptane as the eluents to provide Compound E-3: LCMS m/e 195 (M+H); ¹H NMR (400 MHz, Chloroform-d) δppm 1.55 (d, J=6.69 Hz, 6H), 5.62-5.73 (m, 1H), 6.57 (d, J=3.27 Hz, 1H), 7.44 (d, J=5.37 Hz, 1H), 7.46 (d, J=3.22 Hz, 1H), 7.97 (d, J=5.37 Hz, 1H).

### Preparation of 1-(7-Chloro-1-isopropyl-1H-pyrrolo[2,3-c]pyridin-3-yl)-ethanone (E-4)

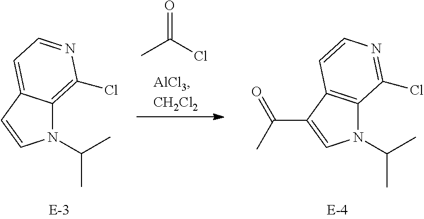

To a solution of AlCl₃ (23.4 g, 175.4 mmol, 2 eq.) in anhydrous CH₂Cl₂ (680 mL) was slowly added a solution of Compound E-3 (17.1 g, 87.7 mmol, 1 eq.) in anhydrous CH₂Cl₂ (200 mL) under an N₂ atmosphere. Acetyl chloride (23.4 g, 175.5 mmol, 2 eq.) was then added slowly and the mixture was stirred at room temperature for 1 hour. After 1 hour, MeOH was added until gas evolution was no longer evident. The volatiles were removed in vacuo and the residue was suspended in CH₂Cl₂ (500 mL). The suspension was filtered, and the filtered material was washed with CH₂Cl₂. The combined organic phases were made basic with the addition of NH₄OH solution. To this was added 200 mL H₂O and the layers were stirred vigorously. The organic phase was separated, and the aqueous phase was extracted three times with CH₂Cl₂. The organic phases were concentrated and the residue was subjected to silica gel chromatography using ethyl acetate and heptane as the eluent to provide Compound E-4: LCMS m/e 237 (M+H); ¹H NMR (400 MHz, Chloroform-d) δppm 1.62 (d, J=6.69 Hz, 6H), 2.55 (s, 3H), 5.64-5.77 (m, 1H), 8.00 (s, 1H), 8.15 (d, J=5.37 Hz, 1H), 8.23 (d, J=5.37 Hz, 1H).

### Preparation of (E)-3-Dimethylamino-1-(7-chloro-1-isopropyl-1H-pyrrolo[2,3-c]pyridin-3-yl)-propenone (E-5)

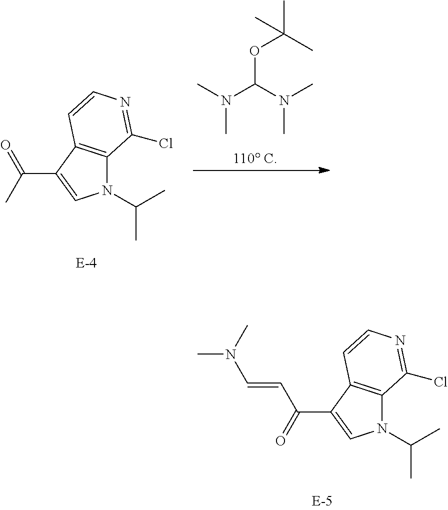

Compound E-4 (18.3 g, 77.4 mmol, 1 eq.) and Bredereck's reagent (t-butoxy bis(dimethylamino)methane, 26.97 g, 154.8 mmol, 2.0 eq.) were combined and the mixture was heated at 110° C. for 2 hours. The cooled residue was dissolved in CH₃OH and CHCl₃ and was then concentrated. The residue was purified by silica gel chromatography ({CHCl₃ 90%, CH₃OH (10%), concentrated aqueous NH₄OH (1%)}:CHCl₃) to provide Compound E-5: LCMS m/e 297 (M+H); ¹H NMR (400 MHz, Chloroform-d) δ ppm 1.59 (d, J=6.64 Hz, 6H), 1.69 (br. s., 1H), 5.55 (d, J=12.35 Hz, 1H), 5.64 -5.75 (m, 1H), 7.79 (d, J=12.35 Hz, 1H), 7.95 (s, 1H), 8.08 (d, J=5.42 Hz, 1H), 8.21 (d, J=5.42 Hz, 1H).

### Preparation of 4-(7-Chloro-1-isopropyl-1H-pyrrolo[2,3-c]pyridin-3-yl)-pyrimidin-2-ylamine (E-6)

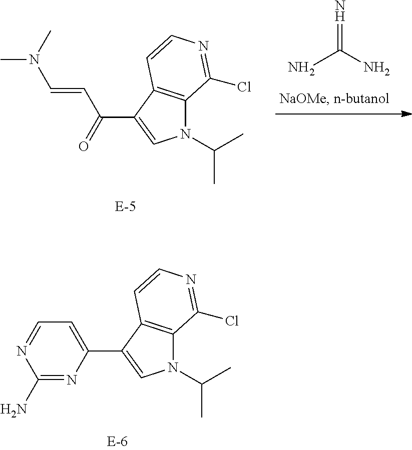

Guanidine HCl (32.7 g, 342 mmol, 10 eq.) was dissolved in n-butanol (250 mL) and cooled to 0° C. To this solution was added NaOMe (18.5 g, 343 mmol, 10 eq.) portionwise and the mixture was stirred at room temperature for 30 min. To this was added a solution of Compound E-5 (10 g, 34.2 mmol, 1 eq.) in n-butanol (250 mL), and the resulting mixture was brought to reflux. After 3 hours, the mixture was cooled and the volatiles were removed by rotary evaporation. To the residue was added H₂O, and the mixture was extracted with CHCl₃ three times. The combined organic phases were washed with H₂O, dried over Na₂SO₄, and concentrated onto Celite. Purification by silica gel chromatography ({CHCl₃ 90%, CH₃OH (10%), concentrated aqueous NH₄OH (1%)}: CHCl₃) provided Compound E-6: LCMS m/e 288 (M+H); ¹H NMR (400 MHz, Chloroform-d) δ ppm 1.63 (d, J=6.69 Hz, 6H), 5.19 (br. s., 2H), 5.69-5.82 (m, 1H), 6.96 (d, J=5.37 Hz, 1H), 8.11 (s, 1H), 8.12 (d, J=5.47 Hz, 1H), 8.24 (d, J=5.42 Hz, 1H), 8.28 (d, J=5.37 Hz, 1H).

### Preparation of 4-(1-Isopropyl-1H-pyrrolo[2,3-c]pyridin-3-yl)-pyrimidin-2-ylamine (E-7)

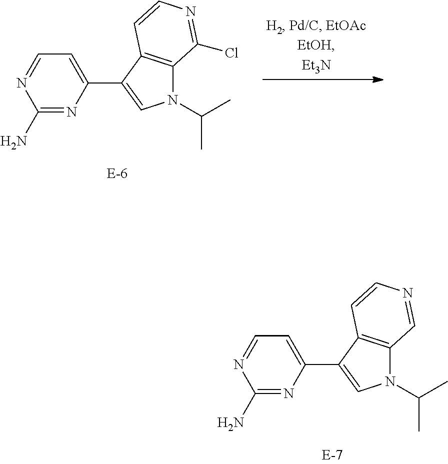

To Compound E-6 (9.2 g, 32 mmol, 1 eq.) in degassed ethyl acetate (250 mL) and EtOH (250 mL) was added 10% Pd/C (1.0 g), followed by Et₃N (10 mL) under an N₂ atmosphere. Hydrogen gas was bubbled through the mixture at room temperature for 22 hours. The mixture was evacuated and purged with N₂ three times, and then filtered through Celite. The filtrate was concentrated onto Celite and the resulting material was purified by silica gel chromatrography using CHCl₃ and (CHCl₃:MeOH:aqueous concentrated NH₄OH [90:10:1]) to provide Compound E-7: LCMS 254 (M+H); ¹H NMR (400 MHz, Chloroform-d) δ ppm 1.66 (d, J=6.69 Hz, 6H), 4.84 (spt, J=6.68 Hz, 1H), 5.03 (br. s., 2H), 6.99 (d, J=5.32 Hz, 1H), 8.07 (s, 1H), 8.18 (dd, J=5.54, 0.95 Hz, 1H), 8.29 (d, J=5.32 Hz, 1H), 8.40 (d, J=5.52 Hz, 1H), 8.89 (d, J=0.83 Hz, 1H).

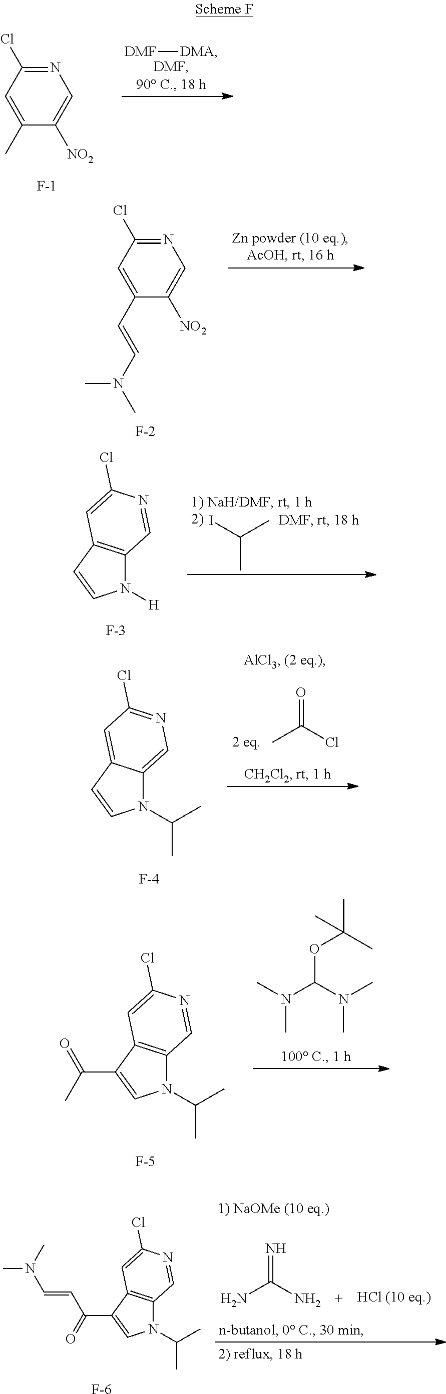

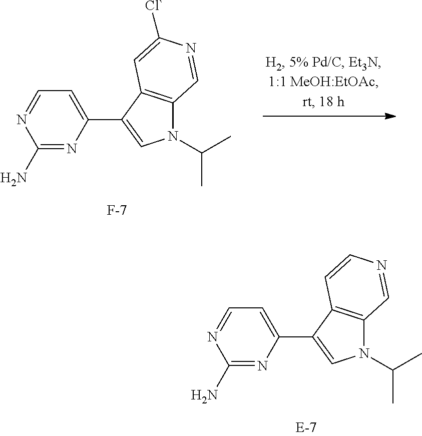

### Preparation of 2-Chloro-4-(2-dimethylamino)ethylene-5-nitropyridine (F-2)

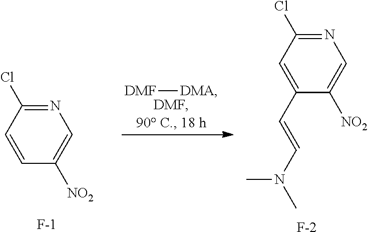

In a 1 L round-bottom flask, 50.01 g (289.8 mmol) 2-chloro-4-methyl-5-nitropyridine F-1 (50.01 g, 289.8 mmol, 1 eq.) was taken up in dry DMF (290 mL). To this was added dimethylformamide dimethyl acetal (84.7 mL, 637.6 mmol, 2.2 eq.). The mixture was heated to 90° C. under an N₂ atmosphere and stirred for 18 hours. The reaction was then cooled to room temperature and the solution was poured into 600 mL of H₂O. The dark orange-red suspension was filtered under vacuum condition, and the solid was collected and dried overnight in a vacuum oven to obtain Compound F-2 as red, powdery, needle-like crystals: LCMS m/e 228 (M+H); ¹H NMR (400 MHz, Chloroform-d) δppm 3.08 (br. s., 6H), 5.97 (d, J=13.28 Hz, 1H), 7.28 (s, 1H), 7.34 (d, J=13.08 Hz, 1H), 8.81 (s, 1H).

### Preparation of 5-Chloro-1H-pyrrolo[2,3-c]pyridine (F-3)

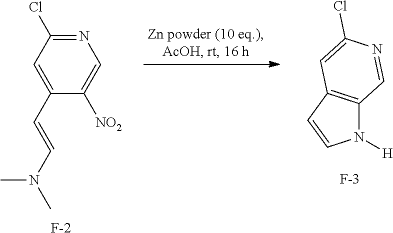

To powdered Zn (43 g, 650 mmol, 10 eq.) in AcOH (650 mL) at 0° C. was added solid F-2 (14.8 g, 65 mmol, 1 eq.). After 16 hours, the mixture was filtered though Celite and the volatiles were removed in vacuo. The residue was mixed with aqueous NaOH solution and was extracted with ethyl acetate three times. The combined organic phases were dried over Na₂SO₄ and concentrated in vacuo. The residue was purified by silica gel chromatography (ethyl acetate:heptane) to provide Compound F-3: LCMS m/e 153 (M+H); ¹H NMR (400 MHz, DMSO-d₆) δ ppm 6.45-6.54 (m, 1H), 7.60 (s, 1H), 7.70 (t, J=2.73 Hz, 1H), 8.55 (s, 1H), 11.76 (br. s., 1H).

### Preparation of 5-Chloro-1-isopropyl-1H-pyrrolo[2,3-c]pyridine (F-4)

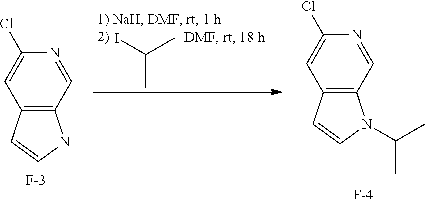

To a solution of 6-azaindole F-3 (608 mg, 4.0 mmol, 1 eq.) in DMF (40 mL) was added NaH (192 mg of a 60% dispersion in mineral oil, 4.8 mmol, 1.2 eq.). The mixture was stirred for 1 hour, and then 2-iodopropane (680 mg, 4.0 mmol, 1 eq.) was added as a solution in DMF. After stirring 18 hours, MeOH was added to quench the reaction and the mixture was concentrated onto Celite. Purification by silica gel chromatography (ethyl acetate:heptane) provided Compound F-4: LCMS m/e 195 (M+H); ¹H NMR (400 MHz, Chloroform-d) δ ppm 1.58 (d, J=6.64 Hz, 6H), 4.74 (spt, J=6.70 Hz, 1H), 6.47 (dd, J=3.22, 0.68 Hz, 1H), 7.42 (d, J=3.32 Hz, 1H), 7.53 (d, J=0.98 Hz, 1H), 8.57 (s, 1H).

### Preparation of 1-(5-Chloro-1-isopropyl-1H-pyrrolo[2,3-c]pyridin-3-yl)-ethanone (F-5)

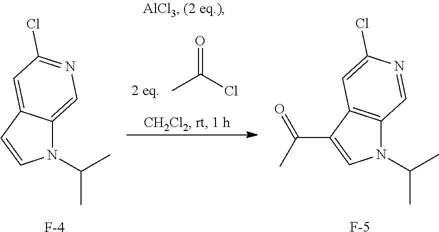

To a solution of AlCl₃ (10.6 g, 79.2 mmol, 2 eq.) in CH₂Cl₂ (300 mL) was added a solution of Compound F-4 (7.7 g, 39.6 mmol, 1 eq.) in CH₂Cl₂ (96 mL) under an N₂ atmosphere. Acetyl chloride (10.6 g, 79.2 mmol, 2 eq.) was then added slowly. After 1 hour, MeOH was added until gas evolution was no longer evident. The volatiles were removed in vacuo and the residue was suspended in CH₂Cl₂ (500 mL). The suspension was filtered and the filtrate was concentrated onto Celite and subjected to silica gel chromatography using MeOH/CH₂Cl₂ as the eluent (gradient 10% MeOH to 40%) to provide Compound F-5: LCMS m/e 237 (M+H); ¹H NMR (400 MHz, Methanol-d₄) δ ppm 1.67 (s, 3H), 1.69 (s, 3H), 2.60 (s, 3H), 4.98-5.11 (m, 1H), 8.49 (s, 1H), 8.96 (s, 1H), 9.21 (s, 1H).

### Preparation of (E)-3-Dimethylamino-1-(5-chloro-1-isopropyl-1H-pyrrolo[2,3-c]pyridin-3-yl)-propenone (F-6)

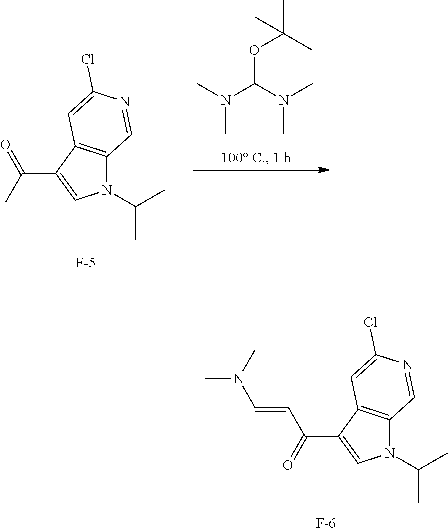

6-azaindole F-5 (300 mg, 1.27 mmol, 1 eq.) was dissolved in Bredereck's reagent (t-butoxy bis(dimethylamino)methane, 2 mL, 9.684 mmol, 7.6 eq.) and the mixture was heated at 100° C. for 1 hour. The reaction mixture was concentrated in vacuo onto Celite and silica gel chromatography (gradient 10 to 50% MeOH in CH₂Cl₂) provided Compound F-6: LCMS m/e 297 (M+5 units); ¹H NMR (400 MHz, Methanol-d₄) δ ppm 1.62 (d, J=6.64 Hz, 6H), 2.91-3.28 (m, 6H), 4.86-4.97 (m, 1H), 5.84 (d, J=12.30 Hz, 1H), 7.76 (d, J=12.30 Hz, 1H), 8.21 (d, J=0.78 Hz, 1H), 8.46 (s, 1H), 8.66 (s, 1H).

### Preparation of 4-(5-Chloro-1-isopropyl-1H-pyrrolo[2,3-c]pyridin-3-yl)-pyrimidin-2-ylamine (F-7)

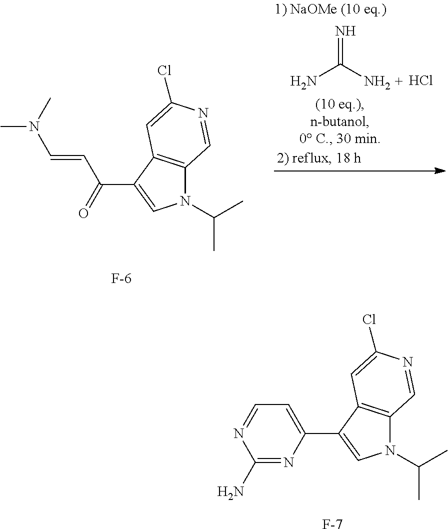

Guanidine HCl (36 g, 377 mmol, 10 eq.) was dissolved in n-butanol (300 mL) and cooled to 0° C. To this solution was added NaOMe (20 g, 377 mmol, 10 eq.) and the mixture was stirred for 30 min. To this was added a solution of Compound F-6 (11 g, 37.7 mmol, 1 eq.) in n-butanol (300 mL) and the resulting mixture was brought to reflux. After 18 hours, the mixture was cooled and the volatiles were removed by rotary evaporation. The residue was dissolved in MeOH and concentrated onto Celite. Column chromatography using silica gel (gradient 10% to 50% MeOH in DCM) provided Compound F-7. LCMS m/e 288 (M+H); ¹H NMR (400 MHz, Methanol-d₄) δ ppm 1.62 (d, J=6.64 Hz, 6H), 2.91-3.28 (m, 6H), 4.86-4.97 (m, 1H), 5.84 (d, J=12.30 Hz, 1H), 7.76 (d, J=12.30 Hz, 1H), 8.21 (d, J=0.78 Hz, 1H), 8.46 (s, 1H), 8.66 (s, 1H).

### Preparation of 4-(1-Isopropyl-1H-pyrrolo[2,3-c]pyridin-3-yl)-pyrimidin-2-ylamine (E-7)

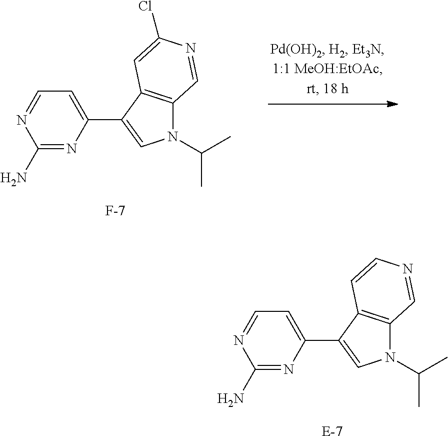

A 1000 mL flask was flushed with N₂ and charged with 10 g of Pd(OH)₂ (20%, wet on carbon, 0.1 eq) followed by a slow addition of 100 mL of clean, dry methanol, further followed by the addition of Et₃N (38.5 mL, 276.3 mmol, 4.0 eq). To this mixture was added a solution of Compound F-7 (19.9 g, 69.1 mmol, 1 eq.) in a 2:1 mixture of ethyl acetate (200 mL) and methanol (100 mL). The flask was then evacuated and charged with H₂ from a balloon three times, and the reaction was allowed to stir at room temperature for 18 hours, recharging the balloon with H₂ as needed. Once the reaction was complete according to LC/MS analysis, the balloon was removed and the flask was evacuated and purged with N₂ for five minutes. The solution was then filtered through Celite, eluting with 1:1 MeOH:EtOAc (approximately 400 mL). The filtered solution was then concentrated under reduced pressure. The product was purified using silica gel chromatography using a gradient beginning at 100% CH₂Cl₂ and finishing with 10% MeOH in CH₂Cl₂ to provide Compound E-7: Spectra as described previously. LC/MS=254 [M +H].

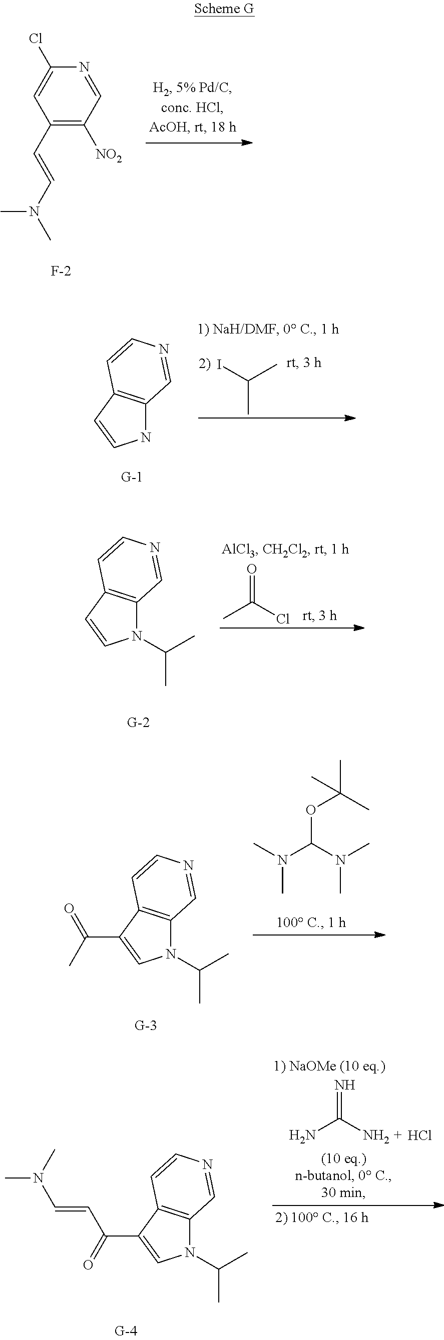

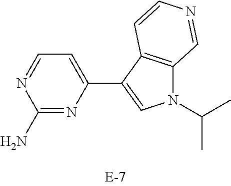

### Preparation of 1H-pyrrolo[2,3-c]pyridine (G-1)

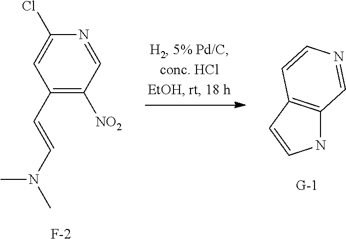

To Compound F-2 (4.55 g, 20 mmol, 1 eq.) in EtOH (200 mL) with 10 drops of 12N aqueous HCl was added 5% Pd/C (1.5 g). The mixture was evacuated and purged with argon three times, and was then evacuated and purged with hydrogen three times. It was left to stir under a hydrogen balloon at for 18 hours. The mixture was then filtered through Celite and the filtrate was concentrated. The residue was purified by silica gel chromatrography using CH₂Cl₂ and (CH₂Cl₂:MeOH:aqueous concentrated NH₄OH [90:10:1]) to provide Compound G-1: LCMS m/e 119 (M+H); ¹H NMR (400 MHz, Chloroform-d) δ ppm 6.68 (d, J=3.12 Hz, 1H), 7.70-7.76 (m, 2H), 8.14 (d, J=6.05 Hz, 1H), 9.16 (s, 1H).

### Preparation of 1-Isopropyl-1H-pyrrolo[2,3-c]pyridine (G-2)

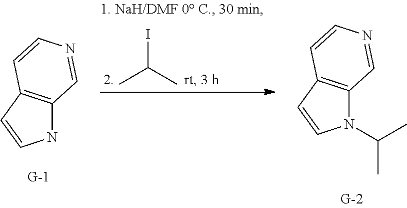

To a 40-mL scintillation vial fitted with a magnetic stir bar was added 6-azaindole G-1 (0.30 g, 2.54 mmol, 1 eq.) and 5 mL anhydrous DMF. Nitrogen was bubbled thoroughly through the solution for 10 minutes. The solution was then cooled to 0° C. followed by the addition of sodium hydride (0.117 g of a 60% dispersion, 2.92 mmol, 1.1 eq.) and the mixture was allowed to stir for 45 minutes. 2-iodopropane (0.240 mL, 2.41 mmol, 0.95 eq.) was then added, and the reaction mixture was allowed to warm to room temperature. After three hours, the crude reaction mixture was concentrated in vacuo onto Celite, and purification by silica gel chromatography (gradient 0 to 10% MeOH in DCM over 12 min) afforded 1-isopropyl-6-azaindole (G-2) as an off-white powder. LCMS m/e 161 (M+H); ¹H NMR (400 MHz, Chloroform-d) δ ppm 1.64 (s, 3H), 1.65 (s, 3H), 4.72-4.93 (m, 1H), 6.98 (d, J=5.3 Hz, 1H), 8.07 (s, 1H), 8.17 (dd, J=5.6, 0.7 Hz, 1H), 8.28 (d, J=5.3 Hz, 1H), 8.38 (d, J=5.5 Hz, 1H), 8.87 (s, 1H).

### Preparation of 1-(1-Isopropyl-1H-pyrrolo[2,3-c]pyridin-3-yl)-ethanone (G-3)

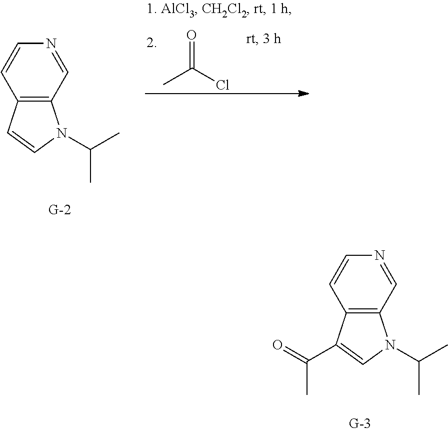

To a 250-mL round bottom flask charged with a magnetic stir bar was added Compound G-2 (0.150 g, 0.940 mmol, 1 eq.) and a suspension of A1Cl₃ (0.627 g, 4.7 mmol, 5 eq.) in CH₂Cl₂ (50 mL) and the mixture was allowed to stir for 1 hour. Acetyl chloride (0.340 mL, 4 7 mmol, 5 eq.) was added dropwise over one minute. After 3 hours of stirring the reaction was quenched with the addition of methanol (10 mL). The reaction mixture was concentrated in vacuo onto Celite, and the residue was subjected to silica gel chromatography (gradient 0 to 10% CH₃OH in CH₂Cl₂) to give 1-(1-Isopropyl-1H-pyrrolo[2,3-c]pyridin-3-yl)-ethanone G-3 as an off-white powder: LCMS m/e 203 (M+H); ¹H NMR (400 MHz, Chloroform-d) δ ppm 1.72 (s, 3H), 1.72 (s, 3H), 2.66 (s, 3H), 8.45 (d, J=5.6 Hz, 1H), 8.72 (d, J=5.5 Hz, 1H), 9.12 (s, 1H), 9.48 (s, 1H).

### Preparation of (E)-3-Dimethylamino-1-(1-isopropyl-1H-pyrrolo[2,3-c]pyridin-3-yl)-propenone (G-4)

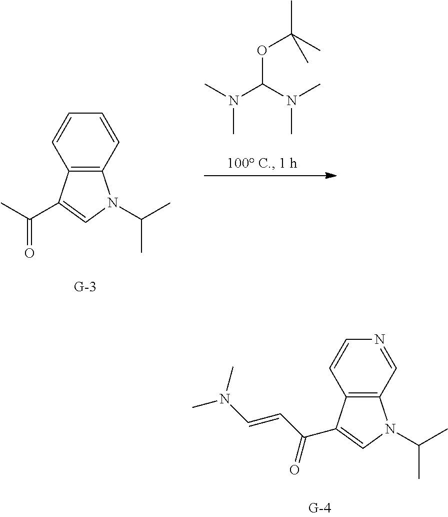

To a 40-mL scintillation vial charged with a magnetic stir bar was added Compound G-3 (400 mg, 1.98 mmol, 1 eq.) and Bredereck's reagent (t-butoxy bis(dimethylamino)methane, 10 mL, 48.4 mmol, 24 eq.). The mixture was heated to 100° C. for 1 hour. After cooling, the reaction mixture was concentrated in vacuo onto Celite and subjected to silica gel chromatography (gradient 0 to 10% CH₃OH in CH₂Cl₂) to provide Compound G-4 as a yellow film. LCMS m/e 258 (M+H). This material was taken on without further characterization.

### Preparation of 4-(1-Isopropyl-1H-pyrrolo[2,3-c]pyridin-3-yl)-pyrimidin-2-ylamine (E-7)

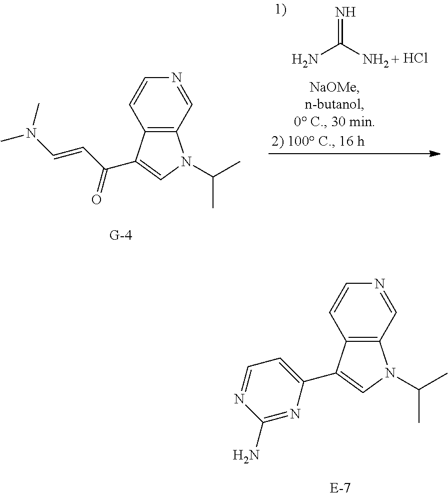

To a 100-mL round bottom flask charged with a magnetic stir bar is added guanidine hydrochloride (0.408 g, 4.27 mmol, 10 eq.) and 1-butanol (1 mL). The mixture was cooled to 0° C., and sodium methoxide (0.231 g, 4.27 mmol, 10 eq.) was added. The mixture was allowed to stir for 30 minutes, then Compound G-4 (0.110 g, 0.427 mmol, 1 eq.) was added and the reaction mixture was stirred for 16 hours at 100° C. After cooling, the resulting mixture was concentrated in vacuo onto Celite and purified by silica gel chromatography (gradient 0 to 10% CH₃OH in CH₂Cl₂) to give 4-(1-Isopropyl-1H-pyrrolo[2,3-c]pyridin-3-yl)-pyrimidin-2-ylamine E-7 as a light yellow powder: LCMS m/e 254 (M+H); ¹H NMR (400 MHz, Chloroform-d) δ ppm 1.65 (s, 3H), 1.67 (s, 3H), 4.74-4.93 (m, 1H), 6.99 (d, J=5.32 Hz, 1H), 8.08 (s, 1H), 8.19 (d, J=5.47 Hz, 1H), 8.29 (d, J=5.32 Hz, 1H), 8.40 (d, J=5.32 Hz, 1H), 8.89 (s, 1H).

### EXAMPLE 1

### N-[4-(1-Isopropyl-1H-pyrrolo[2,3-c]pyridin-3-yl)-pyrimidin-2-yl]-benzamide [11] (Hereinafter Referred to as “Compound [1]”)

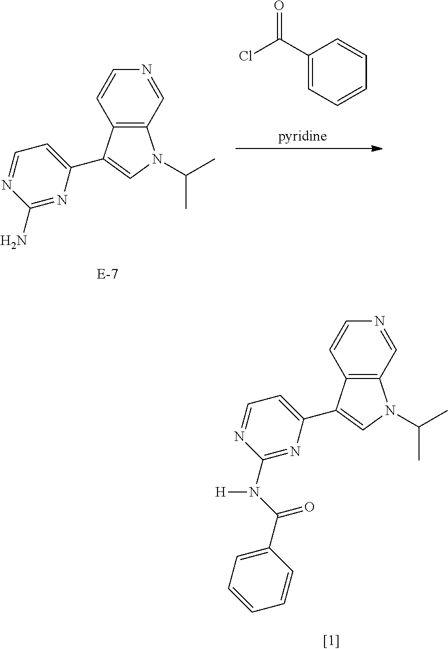

To a 40-mL scintillation vial charged with a magnetic stir bar was added starting material E-7 (50 mg, 0.197 mmol, 1 eq.), pyridine (5 mL), and benzoyl chloride (55 mg, 0.394 mmol, 2 eq). The reaction was stirred at room temperature for 10 minutes, then 5 drops of methanol were added and the mixture concentrated to dryness. The residue was redissolved in methanol with TFA (3 drops) and then purified by reverse phase preparatory HPLC using acetonitrile and water with 0.05% TFA as the eluent to afford Compound [1].

Data for Compound [1]: LCMS m/e 358 (M+H); 1H NMR (400 MHz, Methanol-d₄) δ ppm 1.73 (s, 3H), 1.74 (s, 3H), 5.08-5.19 (m, 1H), 7.56-7.62 (m, 2H), 7.64-7.70 (m, 1H), 7.79 (d, J=5.27 Hz, 1H), 7.98-8.11 (m, 2H), 8.41 (d, J=6.59 Hz, 1H), 8.67 (d, J=5.47 Hz, 1H), 9.26 (s, 1H), 9.41 (s, 1H), 9.68 (d, J=6.30 Hz, 1H).

### EXAMPLE 2

### Thiophene-2-carboxylic acid[4-(1-isopropyl-1H-pyrrolo[2,3-c]pyridin-3-yl)-pyrimidin-2-yl]-amide [2] (Hereinafter Referred to as “Compound [2]”)

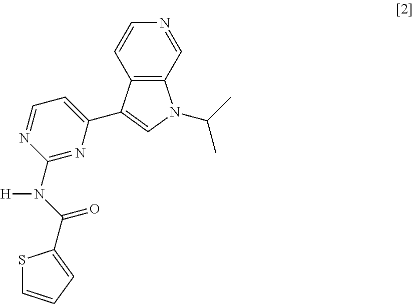

Compound [2] was prepared using a procedure similar to that of Example 1.

Data for Compound [2]: LCMS m/e 364 (M+H); ¹H NMR (400 MHz, Methanol-d₄) δ ppm 1.72 (s, 3H), 1.74 (s, 3H), 5.02-5.22 (m, 1H), 7.23-7.28 (m, 1H), 7.79 (d, J=5.47 Hz, 1H), 7.86 (dd, J=5.00, 1.05 Hz, 1H), 8.06 (dd, J=3.81, 1.07 Hz, 1H), 8.44 (d, J=6.49 Hz, 1H), 8.66 (d, J=5.47 Hz, 1H), 9.26 (s, 1H), 9.41 (s, 1H), 9.67 (d, J=6.49 Hz, 1H).

### EXAMPLE 3

### [4-(1-Isopropyl-1H-pyrrolo[2,3-c]pyridin-3-yl)-pyrimidin-2-yl]-phenyl-amine [3] (Hereinafter Referred to as “Compound [39 ”)

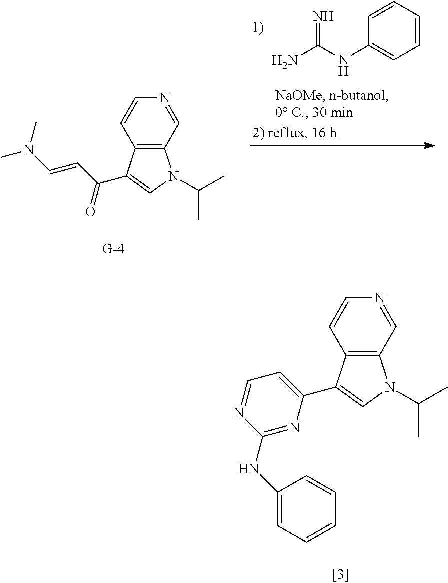

Compound [3] was prepared using a procedure similar to that of the preparation of E-7 from G-4 as shown in Scheme G above.

Data for Compound [3]: LCMS m/e 330 (M+H); ¹H NMR (400 MHz, Methanol-d4) δ ppm 1.71 (s, 3H), 1.73 (s, 3H), 5.07-5.16 (m, 1H), 7.13 (t, J=7.4 Hz, 1H), 7.66 (d, J=7.7 Hz, 2H), 8.30 (d, J=6.6 Hz, 1H), 8.41 (d, J=5.5 Hz, 1H), 9.07 (d, J=6.5 Hz, 1H), 9.16 (s, 1H), 9.39 (s, 1H).

### EXAMPLE 4

### [4-(1-Isopropyl-1H-pyrrolo[2,3-c]pyridin-3-yl)-pyrimidin-2-yl]-p-tolyl-amine[4] (Hereinafter Referred to as “Compound [4]”)

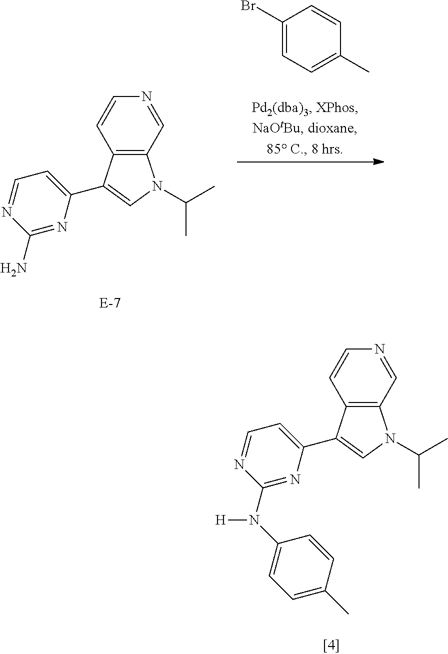

To a 40 mL scintillation vial fitted with a magnetic stir bar was added the starting material E-7 (50 mg, 0.198 mmol, 1 eq.). Nitrogen was bubbled thoroughly through the solution for 10 minutes. Then dioxane (5 mL) was added, followed by the addition of 4-bromotoluene (40 mg, 0.237 mmol, 1.2 eq.) and nitrogen gas was bubbled for an additional 5 min. X-Phos (19 mg, 0.0396 mmol, 0.2 eq.), sodium tert-butoxide (57 mg, 0.594 mmol, 3 eq.), and Pd₂(dba)₃ (9 mg, 0.0099 mmol, 0.05 eq.) were then added sequentially. The reaction was stirred overnight under an N₂ atmosphere at 85° C. The reaction was cooled, and was concentrated to dryness. Then the residue was redissolved in methanol (5 mL), filtered through a 0.2 micron syringe filter, and purified by reverse phase preparatory HPLC using acetonitrile and water with 0.05% TFA as the eluent to provide Compound [4].

Data for Compound [4]: LCMS m/e 344 (M+H); ¹H NMR (400 MHz, Methanol-d₄) δ ppm 1.71 (s, 3H), 1.73 (s, 3H), 2.40 (s, 3H), 5.06-5.21 (m, 1H), 7.27 (d, J=8.10 Hz, 2H), 7.45 (dd, J=5.49, 1.88 Hz, 1H), 7.49 (d, J=8.30 Hz, 2H), 8.33 (t, J=6.66 Hz, 2H), 8.99 (s, 1H), 9.23 (s, 1H), 9.42 (s, 1H).

### EXAMPLE 5

### [4-(1-Isopropyl-1H-pyrrolo[2,3-c]pyridin-3-yl)-pyrimidin-2-yl]-o-tolyl-amine [5] (Hereinafter Referred to as “Compound [5]”)

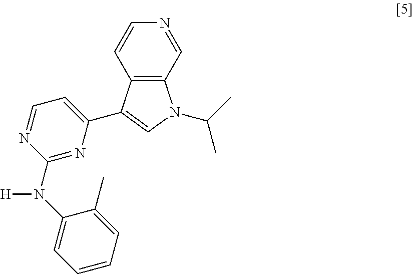

Compound [5] was prepared using a procedure similar to that of Example 4.

Data for Compound [5]: LCMS m/e 344 (M+H); ¹H NMR (400 MHz, Methanol-d₄) δ ppm 1.70 (s, 3H), 1.71 (s, 3H), 2.33 (s, 3H), 5.05-5.17 (m, 1H), 7.34-7.39 (m, 2H), 7.41-7.45 (m, 1H), 7.48 (d, J=6.10 Hz, 2H), 8.19 (d, J=6.44 Hz, 1H), 8.30 (d, J=5.76 Hz, 1H), 8.55 (d, J=5.56 Hz, 1H), 9.26 (s, 1H), 9.40 (s, 1H).

### EXAMPLE 6

### [4-(1-Isopropyl-1H-pyrrolo[2,3-c]pyridin-3-yl)-pyrimidin-2-yl]-m-tolyl-amine [6] (Hereinafter Referred to as “Compound [6]”)

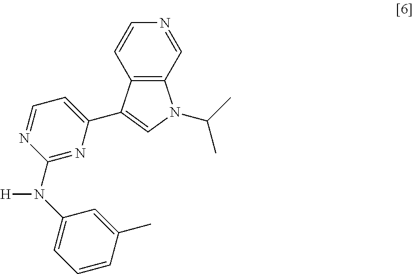

Compound [6] was prepared using a procedure similar to that of Example 4.

Data for Compound [6]: LCMS m/e 344 (M+H); ¹H NMR (400 MHz, Methanol-d₄) δ ppm 1.71 (s, 3H), 1.73 (s, 3H), 2.40 (s, 3H), 5.01-5.21 (m, 1H), 7.05 (d, J=7.47 Hz, 1H), 7.32 (t, J=7.76 Hz, 1H), 7.40 (d, 1H), 7.46 (d, J=5.76 Hz, 1H), 7.49 (s, 1H), 8.32 (d, J=6.44 Hz, 1H), 8.38 (d, J=4.64 Hz, 1H), 9.00 (d, J=5.52 Hz, 1H), 9.23 (s, 1H), 9.42 (s, 1H).

### EXAMPLE 7

### [4-(1-Isopropyl-1H-pyrrolo[2,3-c]pyridin-3-yl)-pyrimidin-2-yl]-(4-methoxy-phenyl)-amine [7] (Hereinafter Referred to as “Compound [7]”)

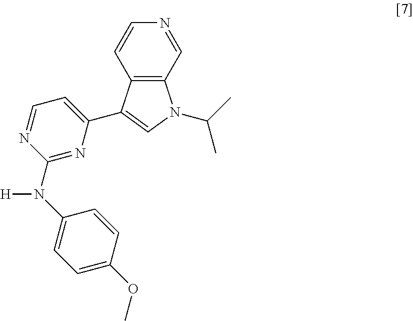

Compound [7] was prepared using a procedure similar to that of Example 4.

Data for Compound [7]: LCMS m/e 360 (M+H); ¹H NMR (400 MHz, Methanol-d₄) δ ppm 1.71 (s, 3H), 1.73 (s, 3H), 3.86 (s, 3H), 5.05-5.24 (m, 1H), 7.04 (d, J=8.98 Hz, 2H), 7.45 (d, J=5.91 Hz, 1H), 7.49 (d, J=8.93 Hz, 2H), 8.32 (s, 1H), 8.94 (s, 1H), 9.25 (s, 1H), 9.43 (s, 1H).

### EXAMPLE 8

### [4-(1-Isopropyl-1H-pyrrolo[2,3-c]pyridin-3-yl)-pyrimidin-2-yl]-(2-methoxy-phenyl)-amine [8] (Hereinafter Referred to as “Compound [8]”)

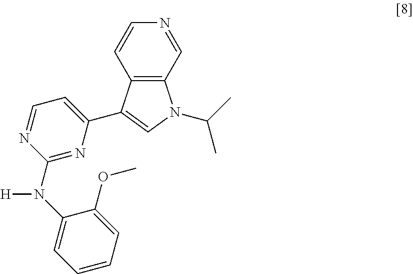

Compound [8] was prepared using a procedure similar to that of Example 4.

Data for Compound [8]: LCMS m/e 360 (M+H); ¹H NMR (400 MHz, Methanol-d₄) δ ppm 1.71 (s, 3H), 1.73 (s, 3H), 3.91 (s, 3H), 5.05-5.18 (m, 1H), 7.08 (m, 1H), 7.16 (d, J=8.20 Hz, 1H), 7.29 (t, J=7.81 Hz, 1H), 7.49 (d, J=5.95 Hz, 1H), 7.89 (d, J=7.91 Hz, 1H), 8.34 (m, 2H), 8.89 (d, J=6.39 Hz, 1H), 9.25 (s, 1H), 9.43 (s, 1H).

### EXAMPLE 9

### [4-(1-Isopropyl-1H-pyrrolo[2,3-c]pyridin-3-yl)-pyrimidin-2-yl]-(3-methoxy-phenyl)-amine [9] (Hereinafter Referred to as “Compound [9]”)

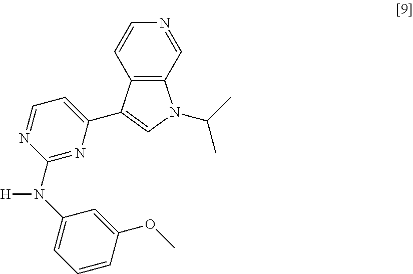

Compound [9] was prepared using a procedure similar to that of Example 4.

Data for Compound [9]: LCMS m/e 360 (M+H); ¹H NMR (400 MHz, Methanol-d₄) δ ppm 1.71 (s, 3H), 1.73 (s, 3H), 3.82 (s, 3H), 5.02-5.20 (m, 1H), 6.69 (dd, J=7.76, 2.00 Hz, 1H), 7.20 (d, 1H), 7.27 (m, 1H), 7.38 (d, J=5.37 Hz, 1H), 7.41 (t, J=2.07 Hz, 1H), 8.30 (d, J=6.54 Hz, 1H), 8.43 (d, J=5.42 Hz, 1H), 9.13 (d, J=6.78 Hz, 1H), 9.15 (s, 1H), 9.39 (s, 1H).

### Example 10

### (2-Fluoro-phenyl)-[4-(1-isopropyl-1H-pyrrolo[2,3-c]pyridin-3-yl)-pyrimidin-2-yl]-amine [10] (Hereinafter Referred to as “Compound [10]”)

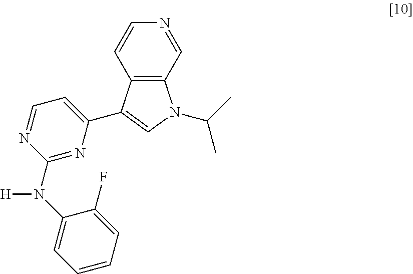

Compound [10] was prepared using a procedure similar to that of Example 4.

Data for Compound [10] LCMS m/e 348 (M+H); ¹H NMR (400 MHz, Methanol-d₄) δ ppm 1.70 (s, 3H), 1.72 (s, 3H), 5.05-5.22 (m, 1H), 7.19-7.33 (m, 3H), 7.45 (d, J=5.66 Hz, 1H), 7.84-7.92 (m, 1H), 8.26 (d, J=6.49 Hz, 1H), 8.41 (d, J=5.56 Hz, 1H), 8.81 (d, J=6.30 Hz, 1H), 9.19 (s, 1H), 9.39 (s, 1H).

### EXAMPLE 11

### (3-Fluoro-phenyl)-[4-(1-isopropyl-1H-pyrrolo[2,3-c]pyridin-3-yl)-pyrimidin-2-yl]-amine [11] (Hereinafter Referred to as “Compound [11]”)

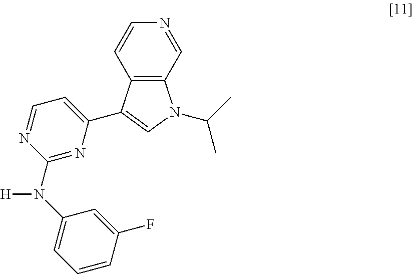

Compound [11] was prepared using a procedure similar to that of Example 4.

Data for Compound [11]: LCMS m/e 348 (M+H); ¹H NMR (400 MHz, Methanol-d₄) δ ppm 1.71 (s, 3H), 1.73 (s, 3H), 5.05-5.20 (m, 1H), 6.80 (m, 1H), 7.31-7.41 (m, 2H), 7.43 (d, J=5.47 Hz, 1H), 7.72 (d, J=11.62 Hz, 1H), 8.34 (d, J=6.54 Hz, 1H), 8.47 (d, J=5.42 Hz, 1H), 9.12 (d, J=6.44 Hz, 1H), 9.17 (s, 1H), 9.41 (s, 1H).

### EXAMPLE 12

### (4-Fluoro-phenyl)-[4-(1-isopropyl-1H-pyrrolo[2,3-c]pyridin-3-yl)-pyrimidin-2-yl]-amine [12] (Hereinafter Referred to as “Compound [12]”)

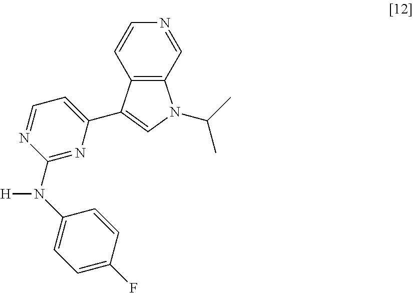

Compound [12] was prepared using a procedure similar to that of Example 4.

Data for Compound [12] LCMS m/e 348 (M+H); ¹H NMR (400 MHz, Methanol-d₄) δ ppm 1.71 (s, 3H), 1.73 (s, 3H), 5.03-5.21 (m, 1H), 7.16 (t, J=8.69 Hz, 2H), 7.43 (dd, J=5.69, 1.10 Hz, 1H), 7.65 (dd, J=9.05, 4.86 Hz, 2H), 8.33 (d, J=6.54 Hz, 1H), 8.39 (d, J=5.66 Hz, 1H), 8.99 (d, J=5.91 Hz, 1H), 9.20 (s, 1H), 9.41 (s, 1H).

### EXAMPLE 13

### (2-Chloro-phenyl)-[4-(1-isopropyl-1H-pyrrolo[2,3-c]pyridin-3-yl)-pyrimidin-2-yl]-amine [13] (Hereinafter Referred to as “Compound [13]”)

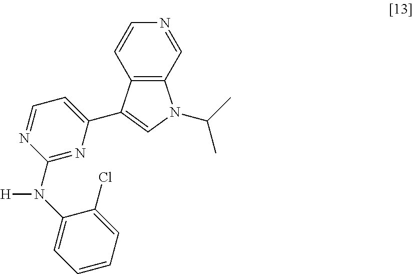

Compound [13] was prepared using a procedure similar to that of Example 4.

Data for Compound [13]: LCMS m/e 364 (M+H); ¹H NMR (400 MHz, Methanol-d₄) δ ppm 1.70 (s, 3H), 1.72 (s, 3H), 5.02-5.21 (m, 1H), 7.27 (t, J=6.96 Hz, 1H), 7.42 (t, J=7.03 Hz, 1H), 7.46 (d, J=5.56 Hz, 1H), 7.56 (dd, J=8.00, 1.42 Hz, 1H), 7.96 (dd, J=8.00, 1.12 Hz, 1H), 8.25 (d, J=6.54 Hz, 1H), 8.42 (d, J=5.47 Hz, 1H), 8.75 (d, J=6.44 Hz, 1H), 9.18 (s, 1H), 9.39 (s, 1H).

### EXAMPLE 14

### (3-Chloro-phenyl)-[4-(1-isopropyl-1H-pyrrolo[2,3-c]pyridin-3-yl)-pyrimidin-2-yl]-amine [14] (Hereinafter Referred to as “Compound [14]”)

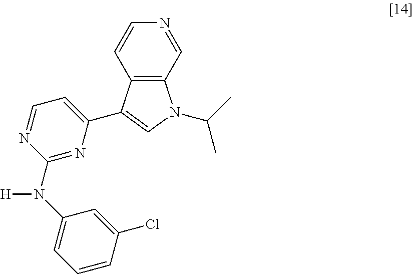

Compound [14] was prepared using a procedure similar to that of Example 4.

Data for Compound [14]: LCMS m/e 364 (M+H); ¹H NMR (400 MHz, Methanol-d₄) δ ppm 1.71 (s, 3H), 1.73 (s, 3H), 5.04-5.25 (m, 1H), 7.06 (dd, J=8.32, 2.32 Hz, 1H), 7.31 (t, J=8.08 Hz, 1H), 7.42 (d, J=5.37 Hz, 1H), 7.48 (dd, J=7.81, 2.29 Hz, 1H), 8.02 (t, J=2.00 Hz, 1H), 8.34 (d, J=6.54 Hz, 1H), 8.47 (d, J=5.32 Hz, 1H), 9.11 (d, J=6.49 Hz, 1H), 9.15 (s, 1H), 9.40 (s, 1H).

### EXAMPLE 15

### (4-Chloro-phenyl)-[4-(1-isopropyl-1H-pyrrolo[2,3-c]pyridin-3-yl)-pyrimidin-2-yl]-amine [15] (Hereinafter Referred to as “Compound [15]”)

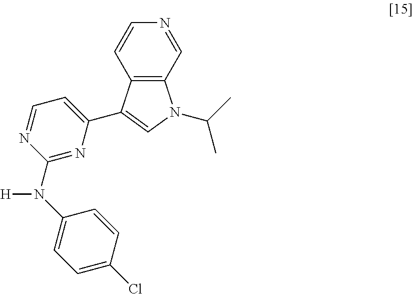

Compound [15] was prepared using a procedure similar to that of Example 4.

Data for Compound [15]: LCMS m/e 364 (M+H); ¹H NMR (400 MHz, Methanol-d₄) δ ppm 1.71 (s, 3H), 1.73 (s, 3H), 5.01-5.23 (m, 1H), 7.35 (d, J=8.83 Hz, 2H), 7.40 (d, J=5.47 Hz, 1H), 7.71 (d, J=8.88 Hz, 2H), 8.34 (d, J=6.54 Hz, 1H), 8.44 (d, J=5.47 Hz, 1H), 9.08 (d, J=6.49 Hz, 1H), 9.16 (s, 1H), 9.40 (s, 1H).

### EXAMPLE 16

### N-[4-(1-Isopropyl-1H-pyrrolo[2,3-c]pyridin-3-yl)-pyrimidin-2-yl]-N′,N′-dimethyl-benzene-1,3-diamine [16] (Hereinafter Referred to as “Compound [16]”)

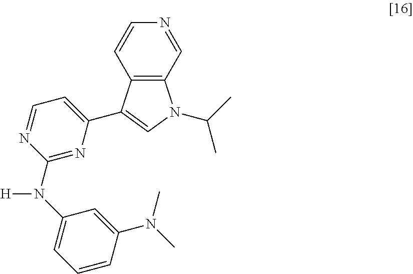

Compound [16] was prepared using a procedure similar to that of Example 4.

Data for Compound [14 LCMS m/e 373 (M+H); ¹H NMR (400 MHz, Methanol-d₄) δ ppm 1.72 (s, 3H), 1.73 (s, 3H), 3.22 (s, 6H), 5.05-5.22 (m, 1H), 7.10 (d, J=6.44 Hz, 1H), 7.48 (d, J=5.52 Hz, 3H), 7.99 (s, 1H), 8.36 (d, J=6.54 Hz, 1H), 8.47 (d, J=5.52 Hz, 1H), 9.17 (d, J=6.49 Hz, 1H), 9.20 (s, 1H), 9.42 (s, 1H).

### EXAMPLE 17

### 4-[4-(1-Isopropyl-1H-pyrrolo[2,3-c]pyridin-3-yl)-pyrimidin-2-ylamino]-benzenesulfonamide [17] (Hereinafter Referred to as “Compound [17]”)

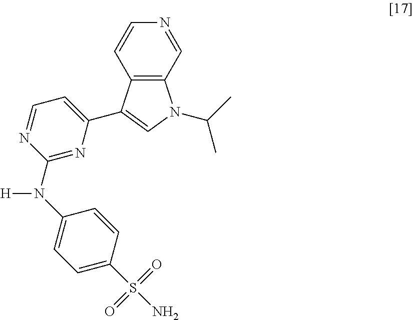

Compound [17] was prepared using a procedure similar to that of Example 4.

Data for Compound [17]: LCMS m/e 409 (M+H); ¹H NMR (400 MHz, Methanol-d₄) δ ppm 1.72 (s, 3H), 1.73 (s, 3H), 5.08-5.17 (m, 1H), 7.45 (d, J=5.32 Hz, 1H), 7.85 (d, 2H), 7.93 (d, 2H), 8.36 (d, J=6.49 Hz, 1H), 8.52 (d, J=5.32 Hz, 1H), 9.12-9.20 (m, 2H), 9.40 (s, 1H).

### EXAMPLE 18

### [4-(1-Isopropyl-1H-pyrrolo[2,3-c]pyridin-3-yl)-pyrimidin-2-yl]-pyridin-2-yl-amine [18] (Hereinafter Referred to as “Compound [18]”)

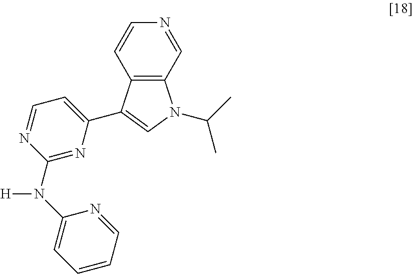

Compound [18] was prepared using a procedure similar to that of Example 4.

Data for Compound [18]: LCMS m/e 331 (M+H); ¹H NMR (400 MHz, Methanol-d₄) δ ppm 1.73 (s, 3H), 1.75 (s, 3H), 5.06-5.25 (m, 1H), 7.44 (t, J=6.74 Hz, 1H), 7.63 (d, J=8.79 Hz, 1H), 7.87 (d, J=5.66 Hz, 1H), 8.32 (m, 1H), 8.43 (dd, J=6.15, 0.98 Hz, 1H), 8.47 (d, J=6.49 Hz, 1H), 8.75 (d, J=5.71 Hz, 1H), 9.29 (d, J=6.49 Hz, 1H), 9.34 (s, 1H), 9.49 (s, 1H).

### EXAMPLE 19

### [4-(1-Isopropyl-1H-pyrrolo[2,3-c]pyridin-3-yl)-pyrimidin-2-yl]-pyridin-3-yl-amine [19] (Hereinafter Referred to as “Compound [19]”)

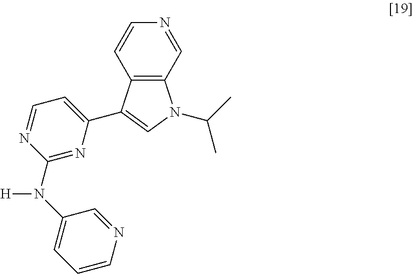

Compound [19] was prepared using a procedure similar to that of Example 4.

Data for Compound [19]: LCMS m/e 331 (M+H); ¹H NMR (400 MHz, Methanol-d₄) δ ppm 1.72 (s, 3H), 1.74 (s, 3H), 5.03-5.25 (m, 1H), 7.61 (d, J=5.42 Hz, 1H), 7.96 (m, 1H), 8.41 (d, J=6.35 Hz, 2H), 8.64 (d, J=5.37 Hz, 1H), 8.68 (d, J=9.57 Hz, 1H), 9.18-9.26 (m, 2H), 9.44 (s, 1H), 9.67 (s, 1H).

### EXAMPLE 20

### (3-Bromo-phenyl)-[4-(1-isopropyl-1H-pyrrolo[2,3-c]pyridin-3-yl)-pyrimidin-2-yl]-amine [20] (Hereinafter Referred to as “Compound [20]”)

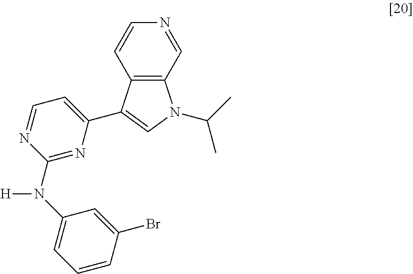

Compound [20] was prepared using a procedure similar to that of Example 4.

Data for Compound [20]: LCMS m/e 409 (M+H); ¹H NMR (400 MHz, Methanol-d₄) δ ppm 1.72 (s, 3H), 1.73 (s, 3H), 5.01-5.22 (m, 1H), 7.21 (d, 1H), 7.27 (t, J=7.91 Hz, 1H), 7.43 (d, J=5.37 Hz, 1H), 7.51 (d, J=8.05 Hz, 1H), 8.18 (t, J=1.83 Hz, 1H), 8.35 (d, J=6.49 Hz, 1H), 8.48 (d, J=5.12 Hz, 1H), 9.10 (d, J=6.49 Hz, 1H), 9.16 (s, 1H), 9.40 (s, 1H).

### EXAMPLE 21

### (3,4-Dichloro-phenyl)-[4-(1-isopropyl-1H-pyrrolo[2,3-c]pyridin-3-yl)-pyrimidin-2-yl]-amine [21] (Hereinafter Referred to as “Compound [21]”)

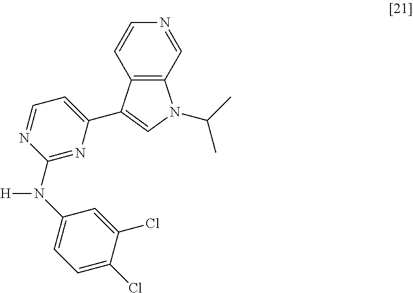

Compound [21] was prepared using a procedure similar to that of Example 4.

Data for Compound [21]: LCMS m/e 398 (M+H); ¹H NMR (400 MHz, Methanol-d₄)δppm 1.72 (s, 3H), 1.73 (s, 3H), 5.03-5.23 (m, 1H), 7.42 (s, 1H), 7.44 (d, J=4.05 Hz, 1H), 7.52 (dd, 1H), 8.20 (s, 1H), 8.36 (d, J=6.54 Hz, 1H), 8.49 (d, J=5.37 Hz, 1H), 9.11 (d, J=6.49 Hz, 1H), 9.15 (s, 1H), 9.41 (s, 1H).

### EXAMPLE 22

### 3-[4-(1-Isopropyl-1H-pyrrolo[2,3-c]pyridin-3-yl)-pyrimidin-2-ylamino]-benzonitrile [22] (Hereinafter Referred to as “Compound [22]”)

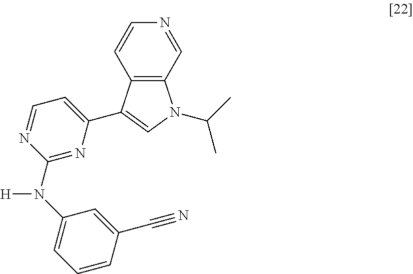

Compound [22] was prepared using a procedure similar to that of Example 4.

Data for Compound [22]: LCMS m/e 355 (M+H); ¹H NMR (400 MHz, Methanol-d₄) δ ppm 1.72 (s, 3H), 1.74 (s, 3H), 7.60-7.70 (m, 3H), 7.84 (d, J=7.52 Hz, 1H), 8.28 (s, 1H), 8.40 (d, J=6.49 Hz, 1H), 8.46 (d, J=6.20 Hz, 1H), 8.85 (d, J=6.35 Hz, 1H), 9.34 (s, 1H), 9.47 (s, 1H).

### EXAMPLE 23

### 4-[4-(1-Isopropyl-1H-pyrrolo[2,3-c]pyridin-3-yl)-pyrimidin-2-ylamino]-benzonitrile [23] (Hereinafter Referred to as “Compound [23]”)

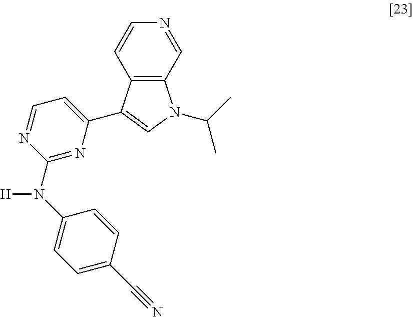

Compound [23] was prepared using a procedure similar to that of Example 4.

Data for Compound [23]: LCMS m/e 355 (M+H); ¹H NMR (400 MHz, Methanol-d₄) δ ppm 1.71 (s, 3H), 1.73 (s, 3H), 5.02-5.22 (m, 1H), 7.47 (d, J=5.37 Hz, 1H), 7.67 (d, J=8.88 Hz, 2H), 7.99 (d, J=8.83 Hz, 2H), 8.38 (d, J=6.20 Hz, 1H), 8.53 (d, J=5.32 Hz, 1H), 9.10 (s, 1H), 9.13 (d, J=6.35 Hz, 1H), 9.36 (s, 2H).

### EXAMPLE 24

### N-[4-(1-Isopropyl-1H-pyrrolo[2,3-c]pyridin-3-yl)-pyrimidin-2-yl]-N′,N′-dimethyl-benzene-1,4-diamine [24] (Hereinafter Referred to as “Compound [24]”)

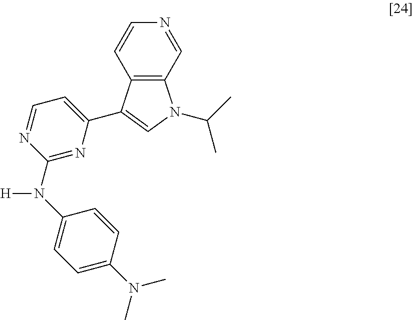

Compound [24] was prepared using a procedure similar to that of Example 4.

Data for Compound [24]: LCMS m/e 373 (M+H); ¹H NMR (400 MHz, Methanol-d₄) δppm 1.72 (d, J=6.64 Hz, 6H), 3.27 (br. s., 6H), 5.06-5.22 (m, 1H), 7.48 (br. s., 2H), 7.91 (br. s., 2H), 8.38 (d, J=6.64 Hz, 1H), 8.48 (br. s., 1H), 9.16 (d, J=6.44 Hz, 1H), 9.19 (s, 1H), 9.42 (s, 1H).

### EXAMPLE 25

### [4-(1-Isopropyl-1H-pyrrolo[2,3-c]pyridin-3-yl)-pyrimidin-2-yl]-(3-trifluoromethyl-phenyl)-amine [25] (Hereinafter Referred to as “Compound [25]”)

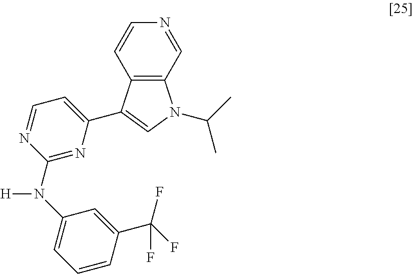

Compound [25] was prepared using a procedure similar to that of Example 4.

Data for Compound [25]: LCMS m/e 398 (M+H); ¹H NMR (400 MHz, Methanol-d₄) δ ppm 1.72 (s, 3H), 1.73 (s, 3H), 5.02-5.27 (m, 1H), 7.34 (d, J=7.76 Hz, 1H), 7.45 (d, J=5.37 Hz, 1H), 7.53 (t, J=7.98 Hz, 1H), 7.84 (d, J=7.08 Hz, 1H), 8.28 (s, 1H), 8.31 (d, J=6.49 Hz, 1H), 8.51 (d, J=5.37 Hz, 1H), 9.06 (d, J=6.44 Hz, 1H), 9.15 (s, 1H), 9.41 (s, 1H).

### EXAMPLE 26

### [4-(1-Isopropyl-1H-pyrrolo[2,3-c]pyridin-3-yl)-pyrimidin-2-yl]-(3-methylsulfanyl-phenyl)-amine [26] (Hereinafter Referred to as “Compound [26]”)

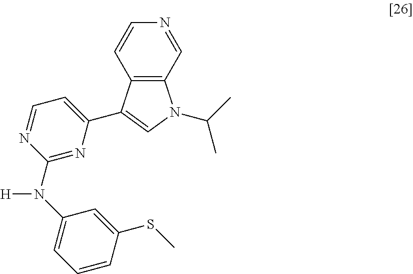

Compound [26] was prepared using a procedure similar to that of Example 4.

Data for Compound [26]: LCMS m/e 376 (M+H); ¹H NMR (400 MHz, Methanol-d₄) δ ppm 1.71 (s, 3H), 1.73 (s, 3H), 2.50 (s, 3H), 5.07-5.22 (m, 1H), 7.26 (dd, J=7.54, 1.44 Hz, 1H), 7.33 (d, J=8.00 Hz, 1H), 7.48 (t, J=7.96 Hz, 1H), 7.55 (s, 1H), 7.70 (d, J=6.78 Hz, 1H), 8.30 (d, J=6.69 Hz, 1H), 8.36 (d, J=6.49 Hz, 1H), 8.76 (s, 1H), 9.42 (s, 1H), 9.48 (s, 1H).

### EXAMPLE 27

### [4-(1-Isopropyl-1H-pyrrolo[2,3-c]pyridin-3-yl)-pyrimidin-2-yl]-(4-methylsulfanyl-phenyl)-amine [27] (Hereinafter Referred to as “Compound [27]”)

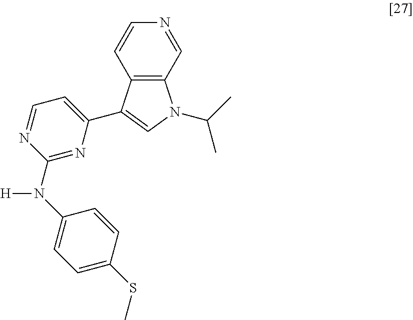

Compound [27] was prepared using a procedure similar to that of Example 4.

Data for Compound [27]: LCMS m/e 376 (M+H); ¹H NMR (400 MHz, Methanol-d₄) δ ppm 1.71 (s, 3H), 1.73 (s, 3H), 2.51 (s, 3H), 5.06-5.18 (m, 1H), 7.35 (d, J=8.74 Hz, 2H), 7.41 (d, J=5.61 Hz, 1H), 7.61 (d, J=8.64 Hz, 2H), 8.32 (d, J=6.49 Hz, 1H), 8.39 (d, J=5.61 Hz, 1H), 9.02 (d, J=6.39 Hz, 1H), 9.19 (s, 1H), 9.41 (s, 1H).

### EXAMPLE 28

### Biphenyl-3-yl-[4-(1-isopropyl-1H-pyrrolo[2,3-c]pyridin-3-yl)-pyrimidin-2-yl]-amine [28] (Hereinafter Referred to as “Compound [28]”)

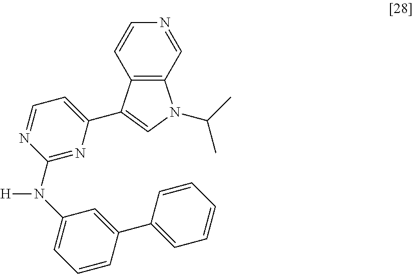

Compound [28] was prepared using a procedure similar to that of Example 4.

Data for Compound [28]: LCMS m/e 406 (M+H); ¹H NMR (400 MHz, Methanol-d₄) δppm 1.68 (s, 3H), 1.70 (s, 3H), 5.02-5.16 (m, 1H), 7.32-7.59 (m, 7H), 7.67 (d, J=7.13 Hz, 2H), 7.85 (br. s., 1H), 8.09 (s, 1H), 8.43 (d, J=5.56 Hz, 1H), 8.90 (d, J=6.30 Hz, 1H), 9.19 (s, 1H), 9.36 (s, 1H).

### EXAMPLE 29

### (3,5-Dimethyl-phenyl)-[4-(1-isopropyl-1H-pyrrolo[2,3-c]pyridin-3-yl)-pyrimidin-2-yl]-amine [29] (Hereinafter Referred to as “Compound [29]”)

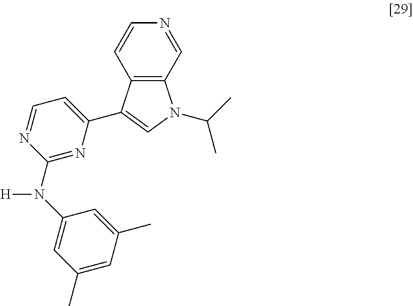

Compound [29] was prepared using a procedure similar to that of Example 4.

Data for Compound [29]: LCMS m/e 358 (M+H); ¹H NMR (400 MHz, Methanol-d₄) δ ppm 1.71 (s, 3H), 1.73 (s, 3H), 2.34 (s, 6H), 5.05-5.19 (m, 1H), 6.87 (s, 1H), 7.25 (s, 2H), 7.43 (d, J=5.76 Hz, 1H), 8.32 (d, J=6.44 Hz, 1H), 8.36 (d, J=5.22 Hz, 1H), 9.00 (d, J=5.81 Hz, 1H), 9.21 (s, 1H), 9.41 (s, 1H).

### EXAMPLE 30

### [4-(1-Isopropyl-1H-pyrrolo[2,3-c]pyridin-3-yl)-pyrimidin-2-yl]-(3-phenoxy-phenyl)-amine [30] (Hereinafter Referred to as “Compound [30]”)

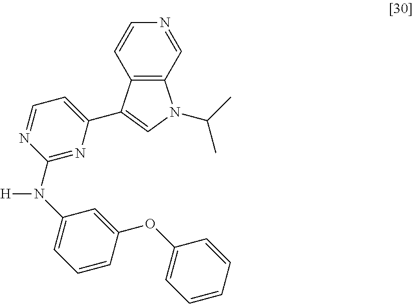

Compound [30] was prepared using a procedure similar to that of Example 4.

Data for Compound [30]: LCMS m/e 422 (M+H); ¹H NMR (400 MHz, Methanol-d₄) δ ppm 1.70 (s, 3H), 1.71 (s, 3H), 5.03-5.19 (m, 1H), 6.69-6.75 (m, 1H), 7.01-7.05 (m, 2H), 7.10 (t, J=7.42 Hz, 1H), 7.31-7.38 (m, 4H), 7.40 (d, J=5.52 Hz, 1H), 7.60-7.64 (m, 1H), 8.15 (d, J=6.49 Hz, 1H), 8.42 (d, J=5.52 Hz, 1H), 9.06 (d, J=6.44 Hz, 1H), 9.15 (s, 1H), 9.39 (s, 1H).

### EXAMPLE 31

### (3,5-Dichloro-phenyl)-[4-(1-isopropyl-1H-pyrrolo[2,3-c]pyridin-3-yl)-pyrimidin-2-yl]-amine [31] (Hereinafter Referred to as “Compound [31]”)

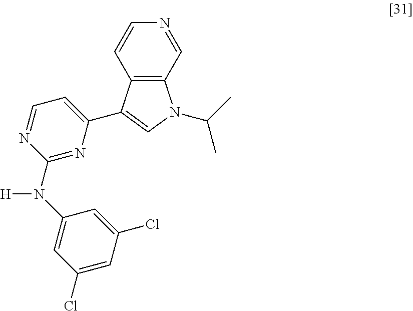

Compound [31] was prepared using a procedure similar to that of Example 4.

Data for Compound [31]: LCMS m/e 398 (M+H); ¹H NMR (400 MHz, Methanol-d₄) δ ppm 1.72 (s, 3H), 1.74 (s, 3H), 5.02-5.25 (m, 1H), 7.04 (t, J=1.83 Hz, 1H), 7.45 (d, J=5.37 Hz, 1H), 7.81 (d, J=1.85 Hz, 2H), 8.36 (d, J=6.54 Hz, 1H), 8.51 (d, J=5.37 Hz, 1H), 9.12 (d, J=6.49 Hz, 1H), 9.15 (s, 1H), 9.41 (s, 1H).

### EXAMPLE 32

### (3-Ethoxy-phenyl)-[4-(1-isopropyl-1H-pyrrolo[2,3-c]pyridin-3-yl)-pyrimidin-2-yl]-amine [32] (Hereinafter Referred to as “Compound [32]”)

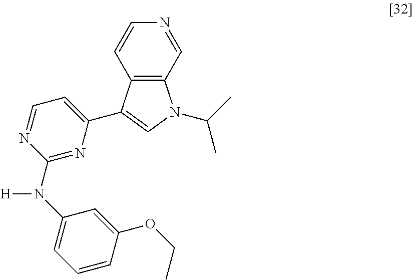

Compound [32] was prepared using a procedure similar to that of Example 4.

Data for Compound [32]: LCMS m/e 374 (M+H); ¹H NMR (400 MHz, Methanol-d₄) δ ppm 1.40 (t, J=6.98 Hz, 3H), 1.71 (s, 3H), 1.73 (s, 3H), 4.07 (q, J=6.98 Hz, 2H), 5.00-5.24 (m, 1H), 6.76 (dd, J=8.22, 1.78 Hz, 1H), 7.15 (dd, J=8.00, 1.17 Hz, 1H), 7.27-7.36 (m, 2H), 7.45 (d, J=5.76 Hz, 1H), 8.32 (d, J=6.54 Hz, 1H), 8.39 (d, J=5.71 Hz, 1H), 9.05 (d, J=6.44 Hz, 1H), 9.21 (s, 1H), 9.41 (s, 1H).

### EXAMPLE 33

### 3-[4-(1-Isopropyl-1H-pyrrolo[2,3-c]pyridin-3-yl)-pyrimidin-2-ylamino]-phenol [33] (Hereinafter Referred to as “Compound [33]”)

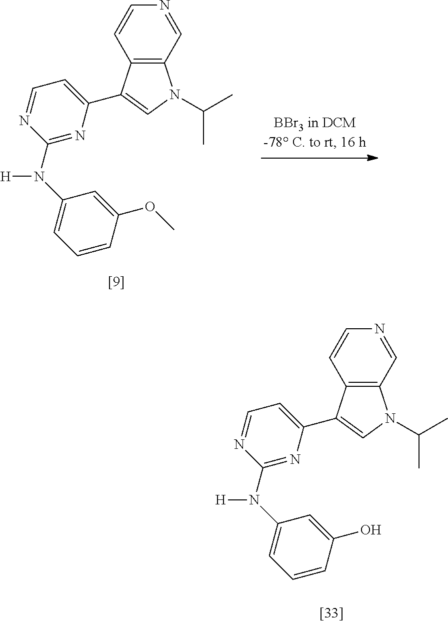

To a 40 mL scintillation vial fitted with a magnetic stir bar was added the starting material Compound [9] (59 mg, 0.164 mmol, 1 eq.). Nitrogen was passed through the vial for 10 minutes and then the reaction vessel cooled to −78° C. and boron tribromide (5 mL of a 1 M solution in CH₂Cl₂, 5 mmol, 30 eq.) was added slowly over 5 minutes. The mixture was allowed to warm to room temperature and then was stirred 16 hours under an N₂ atmosphere. The reaction mixture was quenched with 10 mL methanol concentrated to dryness. The residue was redissolved in 5 mL methanol and purified by reverse-phase preparatory HPLC using acetonitrile and water with 0.05% TFA as the eluent to provide Compound [33].

Data for Compound [33]: LCMS m/e 346 (M+H); ¹H NMR (400 MHz, Methanol-d₄) δ ppm 1.71 (s, 3H), 1.73 (s, 3H), 5.00-5.22 (m, 1H), 6.63 (dd, J=8.13, 2.22 Hz, 1H), 6.97-7.05 (m, 1H), 7.19-7.25 (m, 2H), 8.31 (d, J=6.54 Hz, 1H), 8.38 (d, J=5.71 Hz, 1H), 9.09 (d, J=6.49 Hz, 1H), 9.20 (s, 1H), 9.40 (s, 1H).

### EXAMPLE 34

### 4-[4-(1-Isopropyl-1H-pyrrolo[2,3-c]pyridin-3-yl)-pyrimidin-2-ylamino]-phenol [34] (Hereinafter Referred to as “Compound [34]”)

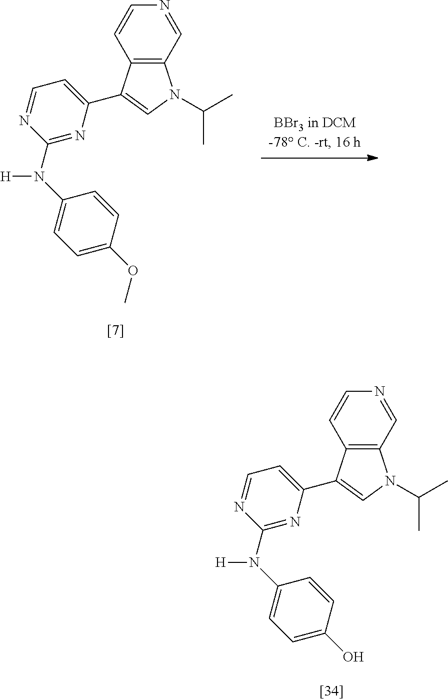

Compound [34] was prepared using a procedure similar to that of Example 33.

Data for Compound [34]: LCMS m/e 346 (M+H); ¹H NMR (400 MHz, Methanol-d₄) δ ppm 1.71 (s, 3H), 1.73 (s, 3H), 5.05-5.26 (m, 1H), 6.93 (d, J=8.74 Hz, 2H), 7.36 (d, J=8.79 Hz, 2H), 7.51 (d, J=6.30 Hz, 1H), 8.25 (d, J=6.25 Hz, 1H), 8.33 (d, J=6.35 Hz, 1H), 8.88 (s, 1H), 9.31 (s, 1H), 9.45 (s, 1H).

### EXAMPLE 35

### [4-(1-Isopropyl-1H-pyrrolo[2,3-c]pyridin-3-yl)-pyrimidin-2-yl]-[3-(4-methyl-piperazin-1-yl)-phenyl]-amine [35] (Hereinafter Referred to as “Compound [35]”)

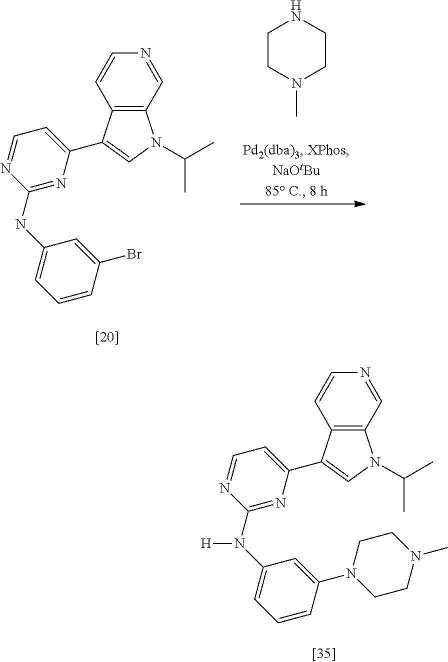

To a 40 mL scintillation vial fitted with a magnetic stir bar was added the starting material Compound [20] (50 mg, 0.198 mmol, 1 eq.). N₂ was bubbled through the solution for 10 minutes. Then 1-methylpiperazine (3 mL) was added and N₂ bubbled for an additional 5 minutes. X-Phos (19 mg, 0.0396 mmol, 0.2 eq.), sodium tert-butoxide (57 mg, 0.594 mmol, 3 eq.), and Pd₂(dba)₃ (9 mg, 0.0099 mmol, 0.05 eq.) were added sequentially, and the mixture was stirred 16 hours under an N₂ atmosphere at 85° C. The mixture was cooled and concentrated to dryness. The residue was redissolved in 5 mL methanol, filtered through a 0.2 micron syringe filter, and purified by reverse-phase preparatory HPLC using acetonitrile and water with 0.05% TFA as the eluent to provide Compound [35].

Data for Compound [351: LCMS m/e 428 (M+H); 1H NMR (400 MHz, Methanol-d₄) δ ppm 1.64 (s, 3H), 1.66 (s, 3H), 2.33 (s, 3H), 2.53-2.61 (m, 4H), 3.17-3.23 (m, 4H), 4.93-5.05 (m, 1H), 6.70 (d, J=7.96 Hz, 1H), 7.14-7.19 (m, 1H), 7.18-7.26 (m, 2H), 7.43 (s, 1H), 8.20 (d, J=5.61 Hz, 1H), 8.30 (d, J=5.42 Hz, 1H), 8.49 (s, 1H), 8.51 (d, J=5.61 Hz, 1H), 8.88 (s, 1H).

### EXAMPLE 36

### [4-(1-Isopropyl-1H-pyrrolo[2,3-c]pyridin-3-yl)-pyrimidin-2-yl]-(4-methanesulfonyl-phenyl)-amine [36] (Hereinafter Referred to as “Compound [36]”)

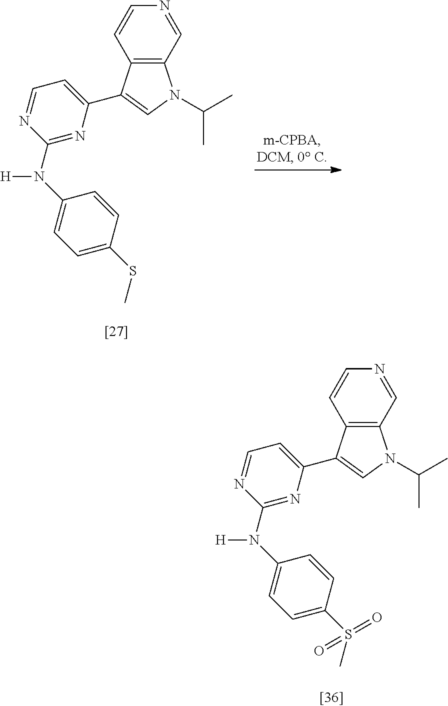

To a 40 mL scintillation vial fitted with a magnetic stir bar was added the starting material Compound [27] (53 mg, 0.140 mmol, 1 eq.) and CH₂Cl₂ (5 mL) and the mixture cooled to 0° C. mCPBA (72.5 mg, 0.420 mmol, 2 eq.) in CH₂Cl₂ (1 mL) was then added dropwise. After stirring for 1 hour, the mixture was worked up by standard procedures and concentrated to dryness. The residue was dissolved in methanol (5 mL) and purified by reverse-phase preparatory HPLC using acetonitrile and water with 0.05% TFA as the gradient to give Compound [36].

Data for Compound [36]: LCMS m/e 408 (M+H); ¹H NMR (400 MHz, Methanol-d₄) δ ppm 1.72 (s, 3H), 1.74 (s, 3H), 3.12 (s, 3H), 5.05-5.24 (m, 1H), 7.48 (d, J=5.37 Hz, 1H), 7.88 (d, J=8.88 Hz, 2H), 8.04 (d, J=8.93 Hz, 2H), 8.38 (d, J=6.54 Hz, 1H), 8.55 (d, J=5.32 Hz, 1H), 9.16 (s, 1H), 9.18 (d, J=6.49 Hz, 1H), 9.41 (s, 1H).

### EXAMPLE 37

### [4-(1-Isopropyl-1H-pyrrolo[2,3-c]pyridin-3-yl)-pyrimidin-2-yl]-(3-methanesulfonyl-phenyl)-amine [37] (Hereinafter Referred to as “Compound [37]”)

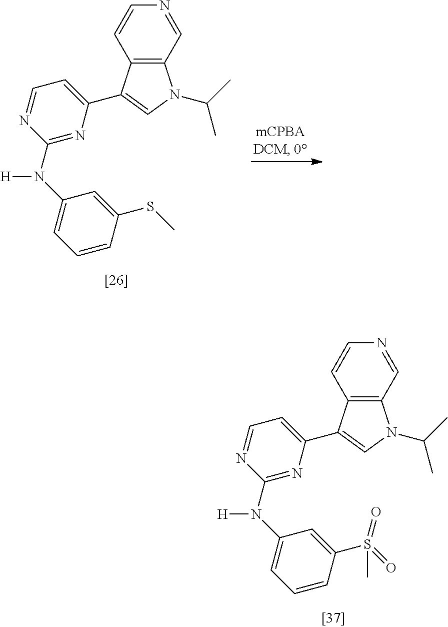

Compound [37] was prepared from Compound [26] using a procedure similar to that of Example 36.

Data for Compound [37]: LCMS m/e 408 (M+H); ¹H NMR (400 MHz, Methanol-d₄) δ ppm 1.73 (s, 3H), 1.75 (s, 3H), 3.15 (s, 3H), 5.01-5.29 (m, 1H), 7.47 (d, J=5.37 Hz, 1H), 7.56-7.65 (m, 2H), 7.77-7.84 (m, 1H), 8.35 (d, J=6.49 Hz, 1H), 8.54 (d, J=5.32 Hz, 1H), 8.75 (d, J=1.66 Hz, 1H), 9.03 (d, J=6.49 Hz, 1H), 9.17 (s, 1H), 9.40 (s, 1H).

### EXAMPLE 38

### [4-(1-Isopropyl-1H-pyrrol[2,3-c]pyridine-3-yl)-pyrimidin-2-yl]-(3-morpholin-4-yl-phenyl)-amine [38] (Hereinafter Referred to as “Compound [38]”)

### (1) 4-(1-Isopropyl-1H-pyrrolo[2,3-c]pyridine-3-yl)-pyrimidin-2-ol (E-7a)

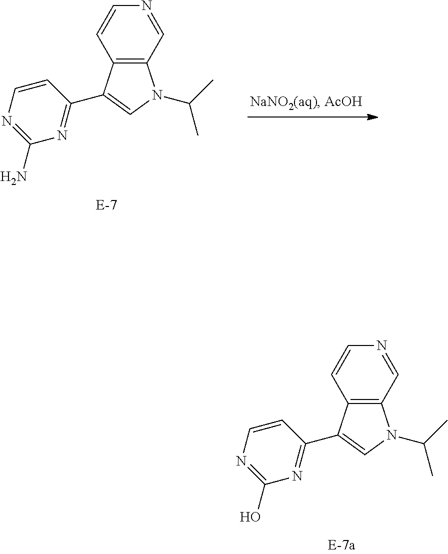

The aminopyrimidine E-7 (0.6276 g, 2.4776 mmol, 1.0 eq) was dissolved in glacial acetic acid (10 mL). Sodium nitrite (0.8548 g, 12.3880 mmol, 5.0 eq) was dissolved in H₂O (6.2 mL) and added to the solution. The mixture was stirred for three hours at room temperature. The solvent was then removed under reduced pressure and the yellow solid was taken back up in methanol and adsorbed onto Celite. Column chromatography using silica gel (gradient 0% to 10% MeOH in CH₂Cl₂) provided Compound E-7a which was taken on to the next step without further characterization:

Data for Compound E-7a: LC/MS m/e 255 (M+H).

### (2) 3-(2-Chloro-pyrimidin-4-yl)-1-isopropyl-1H-pyrrolo[2,3-c]pyridine (E-7b)

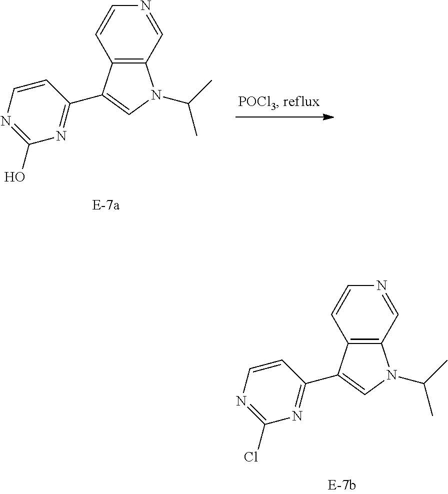

Compound E-7a (0.1420 g, 0.5584 mmol, 1 eq) was dissolved in phosphorous oxychloride (5 mL) and the mixture was heated for 3 hours at 80° C. The reaction was cooled and the solution was slowly added to a stirred solution of 0.01 M HCl. The solution was neutralized with K₂CO₃ and the product was extracted with EtOAc (4×30 mL). The layers were separated and the organic layers were combined, washed with brine and dried over Na₂SO₄. The solution was decanted, and the solvent was removed under reduced pressure. Column chromatography using silica gel (gradient 0% to 10% MeOH in DCM) provided Compound E-7b which was used without further characterization.

Data for Compound E-7b: LC/MS m/e 273 (M+H).

### (3) [4-(1-Isopropyl-1H-pyrrol[2,3-c]pyridine-3-yl)-pyrimidin-2-yl]-(3-morpholin-4-yl-phenyl)-amine [38]

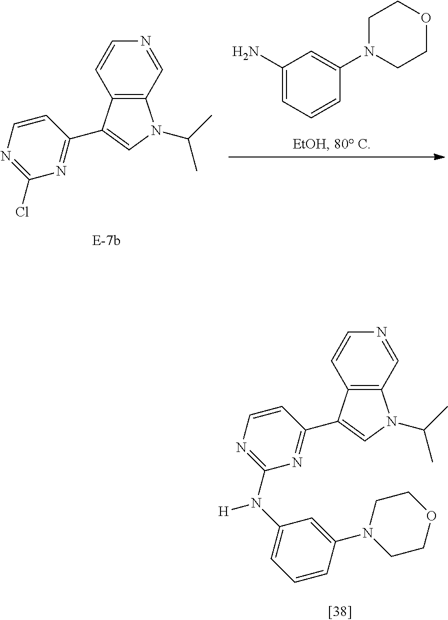

The chloropyrimidine E-7b (26.6 mg, 0.0975 mmol, 1.0 eq) was combined with 3-morpholinoaniline (86.9 mg, 0.4876 mmol, 5.0 eq) in a sealable tube. To this was added ethanol (1.0 mL). The tube was sealed and heated to 80° C. for 2 days. The reaction was cooled and the volatiles were removed under reduced pressure. Purification by reverse phase preparatory HPLC provided Compound [38].

Data for Compound [38]: LC/MS m/e 415 (M+H); ¹H NMR (400 MHz, Methanol-d₄) δ ppm 1.72 (s, 4H), 1.74 (s, 4H), 3.87 (d, J=4.8 Hz, 4H), 5.14 (dt, J=13.3, 6.7 Hz, 1H), 6.99 (dd, J=8.2, 1.8 Hz, 1H), 7.21 (d, J=7.9 Hz, 1H), 7.40 (dt, J=8.1, 4.1 Hz, 1H), 7.46 (s, 1H), 7.51 (d, J=6.0 Hz, 1H), 8.37 (dd, J=9.0, 6.3 Hz, 3H), 9.03 (d, J=6.4 Hz, 1H), 9.27 (s, 1H), 9.45 (s, 1H).

### EXAMPLE 39

### [4-(1-Isopropyl-1H-pyrrolo[2,3-c]pyridine-3-yl)-pyrimidin-2-yl]-(4-morpholin-4-phenyl)-amine [39] (Hereinafter Referred to as “Compound [39]”)

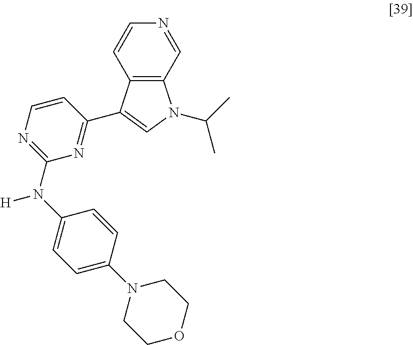

Compound [39] was prepared using a procedure similar to that of Example 38.

Data for Compound [39]: LCMS m/e 415 (M+H); ¹H NMR (400 MHz, Methanol-d₄) δ ppm 1.71 (s, 3H), 1.73 (s, 3H), 3.40 (d, J=4.3 Hz, 3H), 3.95 (d, J=4.8 Hz, 3H), 5.12 (d, J=6.7 Hz, 1H), 7.30 (d, J=8.7 Hz, 2H), 7.53 (d, J=6.1 Hz, 1H), 7.63 (d, J=8.8 Hz, 2H), 8.33 (d, J=6.1 Hz, 1H), 8.36 (d, J=6.5 Hz, 1H), 8.98 (d, J=6.1 Hz, 1H), 9.29 (s, 1H), 9.45 (s, 1H).

### EXAMPLE 40

### 3-[4-(1-Isopropyl-1H-pyrrolo[2,3-c]pyridin-3-yl)-pyrimidin-2-ylamino]benzamide [40] (Hereinafter Referred to as “Compound [40]”)

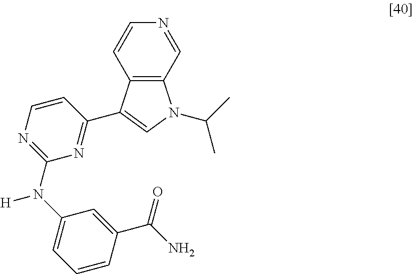

Compound [40] was prepared using a procedure similar to that of Example 38.

Data for Compound [40]: LCMS m/e 373 (M+H); ¹H NMR (400 MHz, Methanol-d₄) δ ppm 1.92 (s, 3H), 1.94 (s, 3H), 5.21 (quin, J=6.7 Hz, 1H), 7.48 (d, J=5.4 Hz, 1H), 7.67 (t, J=7.9 Hz, 1H), 7.77 (d, J=7.8 Hz, 1H), 7.82 (s, 2H), 8.03 (d, J=7.9 Hz, 1H), 8.47 (d, J=5.8 Hz, 1H), 8.52 (s, 1H), 8.63 (d, J=5.3 Hz, 1H), 8.91 (d, J=6.0 Hz, 2H), 9.27 (br. s., 1H).

### EXAMPLE 41

### 3-[4-(1-Isopropyl-1H-pyrrolo[2,3-c]pyridine-3-yl)-pyrimidin-2-ylamino]-4-methoxy-benzamide [41] (Hereinafter Referred to as “Compound [41]”)

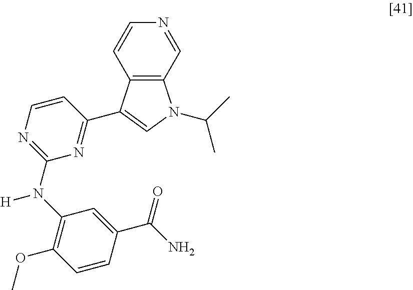

Compound [41] was prepared using a procedure similar to that of Example 38.

Data for Compound [41]: LCMS m/e 403 (M+H); ¹H NMR (400 MHz, Methanol-d₄) δ ppm 1.70 (s, 3H), 1.72 (s, 3H), 3.98 (s, 3H), 5.10 (d, J=6.7 Hz, 1H), 7.19 (d, J=8.6 Hz, 1H), 7.51 (d, J=5.8 Hz, 1H), 7.77 (dd, J=8.6, 2.0 Hz, 1H), 8.28 (d, J=6.4 Hz, 1H), 8.41 (d, J=5.2 Hz, 1H), 8.62 (d, J=1.9 Hz, 1H), 8.85 (d, J=6.4 Hz, 1H), 9.23 (s, 1H), 9.40 (s, 1H).

### EXAMPLE 42-1

### 3-[4-(1-Isopropyl-1H-pyrrolo[2,3-c]pyridine-3-yl)-pyrimidin-2-ylamino]-4-methoxy-benzoic acid methyl ester [42] (Hereinafter Referred to as “Compound [42]”)

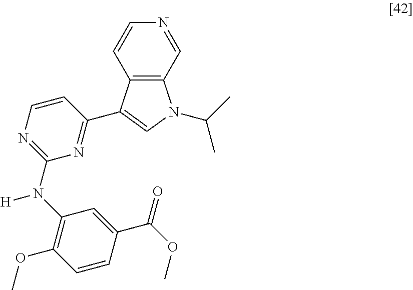

Compound [42] was prepared using a procedure similar to that of Example 38.

Data for Compound [42]: LCMS m/e 418 (M+H); ¹H NMR (400 MHz, DMSO-d₆) δ ppm 1.62 (s, 3H), 1.63 (s, 3H), 3.80 (s, 3H), 3.95 (s, 3H), 5.15 (quin, J=6.6 Hz, 1H), 7.23 (d, J=8.6 Hz, 1H), 7.52 (d, J=5.3 Hz, 1H), 7.77 (d, J=10.6 Hz, 1H), 8.37 (d, J=6.3 Hz, 1H), 8.55 (d, J=3.9 Hz, 1H), 8.70 (d, J=2.0 Hz, 1H), 8.88 (d, J=6.3 Hz, 1H), 9.33 (s, 1H), 9.55 (s, 1H).

### EXAMPLE 42-2

### 3-[4-(1-Isopropyl-1H-pyrrolo[2,3-c]pyridine-3-yl)-pyrimidin-2-ylamino]-4-methoxy-benzoic acid methyl ester [42]

### (1) 3-Guanidino-4-methoxy-benzoic acid methyl ester hydrogen chloride (CII)

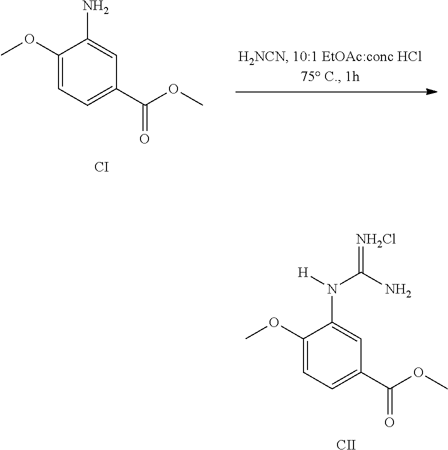

Cyanamide (1.4037 g, 33.3904 mmol, 20 eq) was dissolved in ethyl acetate (15.2 mL). To this was added methyl 3-amino-4-methoxy benzoate CI (0.3025 g, 1.6695 mmol, 1 eq), and the reaction was vigorously stirred for five minutes under an N₂ atmosphere. Concentrated HCl (1.52 mL) was added to the solution, and the reaction mixture was heated to 75° C. and stirred for 1 hour. The reaction was then cooled and filtered, washing sparingly with EtOAc, which yielded Compound CII as a white solid that was carried on without further purification or characterization.

Data for Compound CII: LC/MS m/e 224 (M+H).

### (2) 3-[4-(7-Chloro-1-isoprophy-1H-pyrrolo[2,3-c]pyridine-3-yl)-pyrimidin-2-ylamino]-4-methoxy benzoic acid methyl ester [42a] (Hereinafter Referred to as “Compound [42a]”)

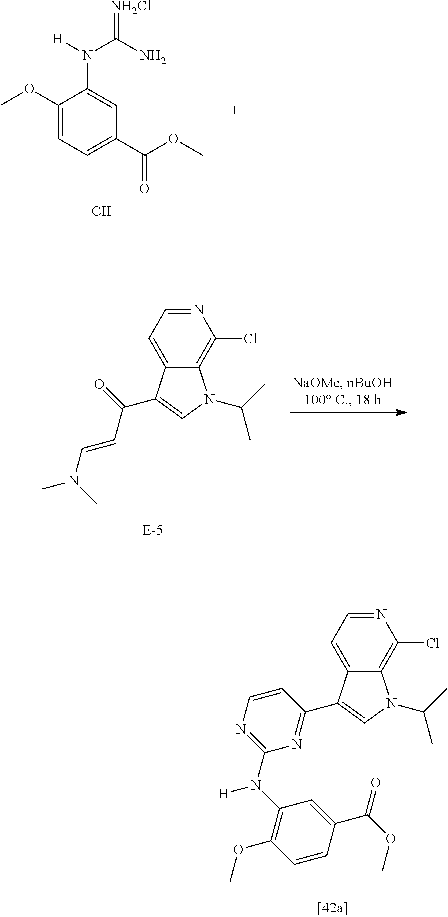

Sodium methoxide (0.2153 g, 3.9840 mmol, 2.0 eq) was suspended in n-butanol (33 mL). To this solution was added the guanidinium CII (0.5173 g, 1.9920 mmol, 1 eq) followed by the azaindole E-5 (0.5812 g, 1.9920 mmol, 1 eq). The reaction was then heated to 100° C. and stirred for 18 hours. The reaction was then cooled and the solvent removed under reduced pressure. The residue was then taken back up in methanol and adsorbed onto Celite under reduced pressure. Purification by silica gel chromatography (0% MeOH to 10% MeOH in CH₂Cl₂) provided Compound [42a] which was used without further characterization.

Data for Compound [42a]: LC/MS m/e 452 (M+H).

### (3) 3-[4-(1-Isopropyl-1H-pyrrolo[2,3-c]pyridine-3-yl)-pyrimidin-2-ylamino]-4-methoxy-benzoic acid methyl ester [42]

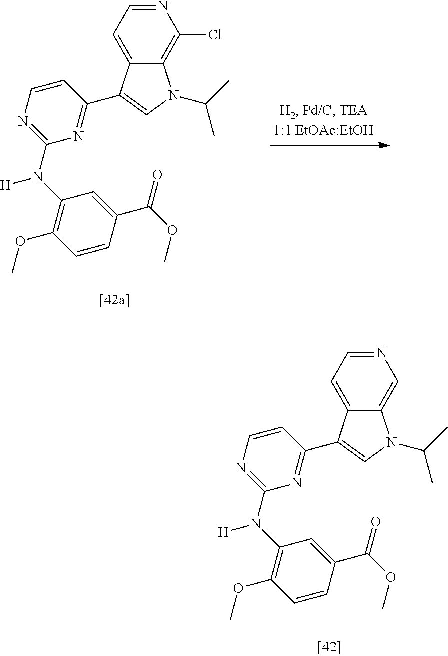

10% Palladium on carbon (0.011 g, 0.1 eq) was taken up in ethanol (5 mL) under an inert atmosphere of N₂. To this was added the chloro-azaindole [42a] (0.1084 g, 0.2399 mmol, 1 eq) dissolved in ethyl acetate (5 mL). To this mixture was then added triethylamine (0.200 mL, 1.4392 mmol, 6.0 eq), and the flask was evacuated and purged with H₂ from a balloon three times. The reaction was stirred under a H₂ atmosphere for 18 hours, recharging the hydrogen as needed. The reaction flask was then evacuated and flushed with N₂. The reaction mixture was filtered through Celite, eluting with EtOAc. The volatiles were then removed under reduced pressure, and the residue purified by silica gel chromatography (gradient 0% MeOH to 7% MeOH in DCM) to afford Compound [42] (spectral data as described previously) which was carried on to the next step without further purification or characterization.

### EXAMPLE 43

### 4-[4-(1-Isopropyl-1H-pyrrolo[2,3-c]pyridine-3-yl)-pyrimidin-2-ylamino]-benzamide [43] (Hereinafter Referred to as “Compound [43]”)

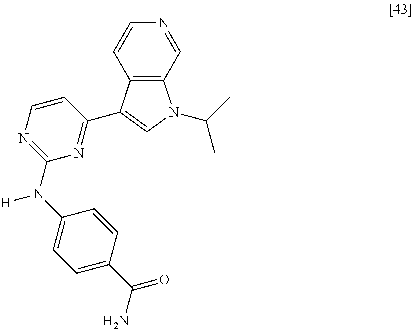

Compound [43] was prepared using a procedure similar to that of Example 38.

Data for Compound [43]: LCMS m/e 373 (M+H); ¹H NMR (400 MHz, Methanol-d₄) δ ppm 1.74 (s, 3H), 1.75 (s, 3H), 5.15 (quin, J=6.7 Hz, 1H), 7.46 (d, J=5.4 Hz, 1H), 7.83-7.96 (m, 4H), 8.40 (d, J=6.5 Hz, 1H), 8.53 (d, J=5.3 Hz, 1H), 9.16-9.21 (m, 2H), 9.42 (s, 1H).

### EXAMPLE 44

### 5-[4-(1-Isopropyl-1H-pyrrolo[2,3-c]pyridin-3-yl)-pyrimidin-2-ylamino]-nicotinonitrile [44] (Hereinafter Referred to as “Compound [44]”)

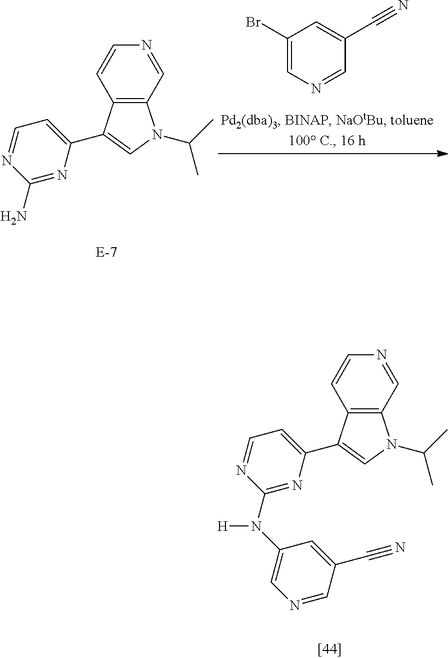

To a 40 mL scintillation vial fitted with a magnetic stir bar was added the starting material E-7 (50 mg, 0.198 mmol, 1 eq.) and N₂ was bubbled through the vessel for 10 minutes. To the vessel was added toluene (5 mL) followed by the addition of 3-bromo-5-cyanopyridine (43 mg, 0.237 mmol, 1.2 eq.), and N₂ was bubbled through the solution for an additional 5 minutes. BINAP (12 mg, 0.0198 mmol, 0.10 eq.), sodium tert-butoxide (57 mg, 0.594 mmol, 3 eq.), and Pd₂(dba)₃ (9 mg, 0.0099 mmol, 0.05 eq.) were then added sequentially. The reaction was stirred for 16 hours under an N₂ atmosphere at 100° C. The reaction mixture was then cooled, and was concentrated to dryness. The residue was re-dissolved in methanol (5 mL), then filtered through a 0.2 micron syringe filter, and purified by reverse-phase preparatory HPLC using acetonitrile and water with 0.05% TFA as the eluent to provide Compound [44].

Data for Compound [44]: LCMS m/e 356 (M+H); ¹H NMR (400 MHz, Methanol-d₄) δ ppm 1.72 (s, 3H), 1.74 (s, 3H), 5.05-5.24 (m, 1H), 7.52 (d, J=5.32 Hz, 1H), 8.40 (d, J=6.49 Hz, 1H), 8.50 (d, J=1.46 Hz, 1H), 8.59 (d, J=5.37 Hz, 2H), 8.88-8.94 (m, 1H), 9.03 (d, J=2.34 Hz, 1H), 9.14 (d, J=6.49 Hz, 1H), 9.18 (s, 1H), 9.43 (s, 1H).

### EXAMPLE 45

### 3-Bromo-5-[4-(1-isopropyl-1H-pyrrolo[2,3-c]pyridin-3-yl)-pyrimidin-2-ylamino]-benzonitrile [45] (Hereinafter Referred to as “Compound [45]”)

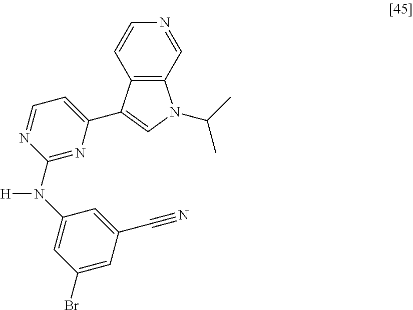

Compound [45] was prepared using a procedure similar to that of Example 44.

Data for Compound [45]: LCMS m/e 434 (M+H); ¹H NMR (400 MHz, Methanol-d₄) δ ppm 1.72 (s, 3H), 1.74 (s, 3H), 5.06-5.24 (m, 1H), 7.45-7.55 (m, 2H), 8.20-8.26 (m, 1H), 8.32 (t, J=1.85 Hz, 1H), 8.39 (d, J=6.44 Hz, 1H), 8.56 (d, J=5.27 Hz, 1H), 9.11 (d, 1H), 9.16 (s, 1H), 9.42 (s, 1H).

### EXAMPLE 46

### 3-[4-(1-Isopropyl-1H-pyrrolo[2,3-c]pyridin-3-yl)-pyrimidin-2-ylamino]-4-methoxy-benzonitrile [46] (Hereinafter Referred to as “Compound [46]”)

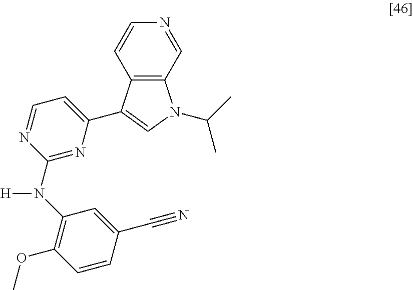

Compound [46] was prepared using a procedure similar to that of Example 44.

Data for Compound [46]: LCMS m/e 385 (M+H); ¹H NMR (400 MHz, Methanol-d₄) δ ppm 1.72 (s, 3H), 1.74 (s, 3H), 3.99 (s, 3H), 5.06-5.24 (m, 1H), 7.35 (d, J=8.64 Hz, 1H), 7.73 (d, J=6.64 Hz, 1H), 7.78 (dd, J=8.59, 2.05 Hz, 1H), 8.25 (d, J=1.51 Hz, 1H), 8.40 (t, J=6.88 Hz, 2H), 8.74 (d, J=5.95 Hz, 1H), 9.40 (s, 1H), 9.49 (s, 1H).

### EXAMPLE 47

### [4-(1-Isopropyl-1H-pyrrolo[2,3-c]pyridin-3-yl)-pyrimidin-2-yl]-[3-(4-methyl-piperazin-1-ylmethyl)-phenyl]-amine [47] (Hereinafter Referred to as “Compound [47]”)

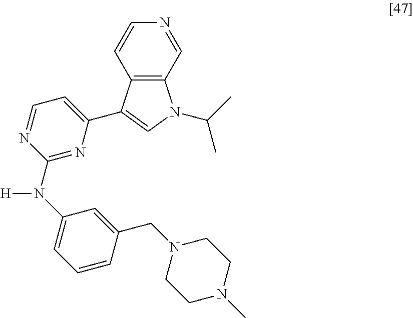

Compound [47] was prepared using a procedure similar to that of Example 44.

Data for Compound [47]: LCMS m/e 442 (M+H); ¹H NMR (400 MHz, Methanol-d₄) δ ppm 1.65 (s, 3H), 1.66 (s, 3H), 2.25 (s, 3H), 2.48 (br. s., 8H), 3.57 (s, 2H), 4.93-5.13 (m, 1H), 7.04 (d, J=7.61 Hz, 1H), 7.24 (d, J=5.42 Hz, 1H), 7.31 (t, J=7.81 Hz, 1H), 7.62 (dd, J=8.05, 1.27 Hz, 1H), 7.71 (s, 1H), 8.19 (d, J=5.61 Hz, 1H), 8.32 (d, J=5.37 Hz, 1H), 8.49 (d, J=0.54 Hz, 1H), 8.50 (s, 1H), 8.89 (s, 1H).

### EXAMPLE 48

### [4-(1-Isopropyl-1H-pyrrolo[2,3-c]pyridin-3-yl)-pyrimidin-2-yl]-(2-methoxy-5-methyl-phenyl)-amine [48] (Hereinafter Referred to as “Compound [48]”)

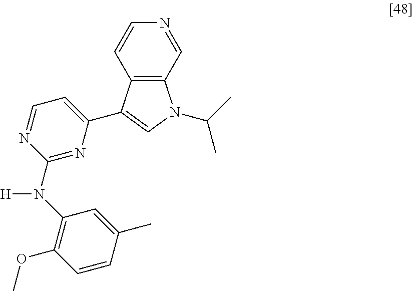

Compound [48] was prepared using a procedure similar to that of Example 44.

Data for Compound [48]: LCMS m/e 374 (M+H); ¹H NMR (400 MHz, Methanol-d₄) δ ppm 1.72 (s, 3H), 1.73 (s, 3H), 2.35 (s, 3H), 3.88 (s, 3H), 5.05-5.26 (m, 1H), 7.03-7.07 (m, 1H), 7.12 (dd, 1H), 7.51 (d, J=6.05 Hz, 1H), 7.70 (d, J=1.61 Hz, 1H), 8.34 (t, J=6.25 Hz, 2H), 8.90 (d, J=6.35 Hz, 1H), 9.28 (s, 1H), 9.45 (s, 1H).

### EXAMPLE 49

### (5-Fluoro-2-methoxy-phenyl)-[4-(1-isopropyl-1H-pyrrolo[2,3-c]pyridin-3-yl)-pyrimidin-2-yl]-amine [49] (Hereinafter Referred to as “Compound [49]”)

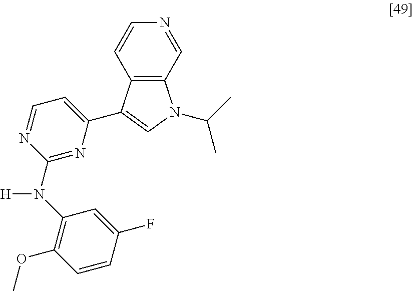

Compound [49] was prepared using a procedure similar to that of Example 44.

Data for Compound [49]: LCMS m/e 378 (M+H); ¹H NMR (400 MHz, Methanol-d₄) δ ppm 1.72 (s, 3H), 1.73 (s, 3H), 3.94 (s, 3H), 4.97-5.21 (m, 1H), 6.75-6.86 (m, 1H), 7.03 (dd, J=9.01, 5.05 Hz, 1H), 7.45 (d, J=5.47 Hz, 1H), 8.13 (dd, J=10.84, 3.07 Hz, 1H), 8.35 (d, J=6.54 Hz, 1H), 8.48 (d, J=5.52 Hz, 1H), 8.97 (d, J=6.49 Hz, 1H), 9.17 (s, 1H), 9.41 (s, 1H).

### EXAMPLE 50

### (2,5-Dimethoxy-phenyl)-[4-(1-isopropyl-1H-pyrrolo[2,3-c]pyridin-3-yl)-pyrimidin-2-yl]-amine [50] (Hereinafter Referred to as “Compound [50]”)

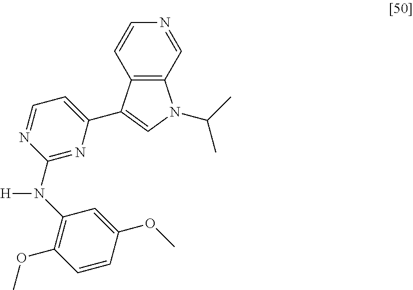

Compound [50] was prepared using a procedure similar to that of Example 44.

Data for Compound [50]: LCMS m/e 390 (M+H); ¹H NMR (400 MHz, Methanol-d₄) δ ppm 1.71 (s, 3H), 1.73 (s, 3H), 3.79 (s, 3H), 3.88 (s, 3H), 5.06-5.19 (m, 1H), 6.78 (dd, J=8.93, 3.03 Hz, 1H), 7.04 (d, J=8.98 Hz, 1H), 7.47 (d, J=5.76 Hz, 1H), 7.71 (d, J=3.03 Hz, 1H), 8.33 (d, J=6.49 Hz, 1H), 8.39 (d, J=5.76 Hz, 1H), 8.95 (d, J=6.49 Hz, 1H), 9.22 (s, 1H), 9.42 (s, 1H).

### EXAMPLE 51

### [4-(1-Isopropyl-1H-pyrrolo[2,3-c]pyridin-3-yl)-pyrimidin-2-yl]-(2-trifluoromethoxy-phenyl)-amine [51] (Hereinafter Referred to as “Compound [51]”)

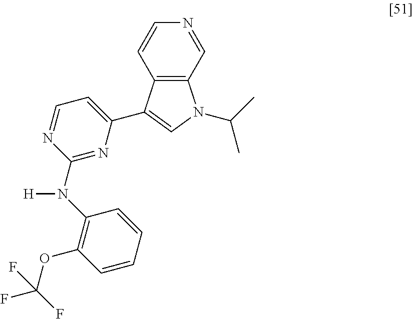

Compound [51] was prepared using a procedure similar to that of Example 44.

Data for Compound [51]: LCMS m/e 414 (M+H); ¹H NMR (400 MHz, Methanol-d₄) δ ppm 1.70 (s, 3H), 1.72 (s, 3H), 5.02-5.24 (m, 1H), 7.31-7.38 (m, 1H), 7.42-7.50 (m, 3H), 8.01 (dd, J=8.15, 1.32 Hz, 1H), 8.27 (d, J=6.49 Hz, 1H), 8.42 (d, J=5.56 Hz, 1H), 8.76 (d, J=5.66 Hz, 1H), 9.18 (s, 1H), 9.40 (s, 1H).

### EXAMPLE 52

### (3,5-Difluoro-2-methoxy-phenyl)-[4-(1-isopropyl-1H-pyrrolo[2,3-c]pyridin-3-yl)-pyrimidin-2-yl]-amine [52] (Hereinafter Referred to as “Compound [52]”)

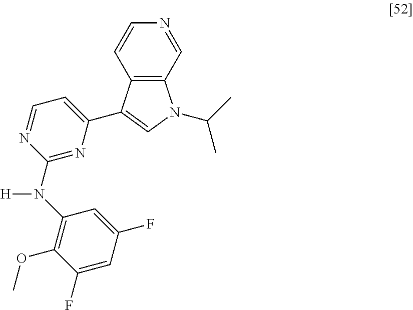

Compound [52] was prepared using a procedure similar to that of Example 44.

Data for Compound [54 LCMS m/e 396 (M+H); ¹H NMR (400 MHz, Methanol-d₄) δ ppm 1.72 (s, 3H), 1.74 (s, 3H), 5.04-5.24 (m, 1H), 6.58-6.74 (m, 1H), 7.49 (d, J=5.37 Hz, 1H), 8.08 (d, J=5.91 Hz, 1H), 8.36 (d, J=6.49 Hz, 1H), 8.50-8.56 (m, 1H), 8.98-9.04 (m, 1H), 9.16 (s, 1H), 9.42 (s, 1H).

### EXAMPLE 53

### (5-Chloro-2-methoxy-phenyl)-[4-(1-isopropyl-1H-pyrrolo[2,3-c]pyridin-3-yl)-pyrimidin-2-yl]-amine [53] (Hereinafter Referred to as “Compound [53]”)

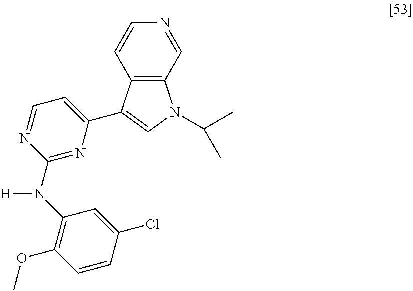

Compound [53] was prepared using a procedure similar to that of Example 44.

Data for Compound [153]: LCMS m/e 394 (M+H); ¹H NMR (400 MHz, Methanol-d₄) δ ppm 1.72 (s, 3H), 1.74 (s, 3H), 5.04-5.24 (m, 1H), 6.58-6.74 (m, 1H), 7.49 (d, J=5.37 Hz, 1H), 8.08 (d, J=5.91 Hz, 1H), 8.36 (d, J=6.49 Hz, 1H), 8.50-8.56 (m, 1H), 8.98-9.04 (m, 1H), 9.16 (s, 1H), 9.42 (s, 1H).

### EXAMPLE 54

### (4-Chloro-2-methoxy-phenyl)-[4-(1-isopropyl-1H-pyrrolo[2,3-c]pyridin-3-yl)-pyrimidin-2-yl]-amine [54] (Hereinafter Referred to as “Compound [54]”)

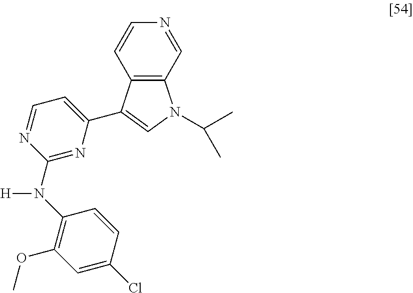

Compound [54] was prepared using a procedure similar to that of Example 44.

Data for Compound [54]: LCMS m/e 394 (M+H); ¹H NMR (400 MHz, Methanol-d₄) δ ppm 1.71 (s, 3H), 1.73 (s, 3H), 3.93 (s, 3H), 5.03-5.18 (m, 1H), 7.05 (dd, J=8.54, 2.25 Hz, 1H), 7.14 (d, J=2.25 Hz, 1H), 7.45 (d, J=5.66 Hz, 1H), 8.03 (d, J=8.54 Hz, 1H), 8.34 (d, J=6.49 Hz, 1H), 8.40 (d, J=5.66 Hz, 1H), 8.90 (d, J=6.49 Hz, 1H), 9.19 (s, 1H), 9.42 (s, 1H).

### EXAMPLE 55

### (3,4-Difluoro-2-methoxy-phenyl)-[4-(1-isopropyl-1H-pyrrolo[2,3-c]pyridin-3-yl)-pyrimidin-2-yl]-amine [55] (Hereinafter Referred to as “Compound [55]”)

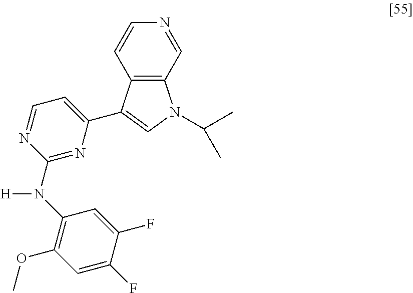

Compound [55] was prepared using a procedure similar to that of Example 44.

Data for Compound [55]: LCMS m/e 396 (M+H); ¹H NMR (400 MHz, Methanol-d₄) δ ppm 1.72 (s, 3H), 1.73 (s, 3H), 3.93 (s, 3H), 5.02-5.23 (m, 1H), 7.07 (dd, J=12.10, 7.42 Hz, 1H), 7.45 (d, J=5.52 Hz, 1H), 8.23 (dd, J=12.76, 8.71 Hz, 1H), 8.35 (d, J=6.54 Hz, 1H), 8.47 (d, J=5.47 Hz, 1H), 8.97 (d, J=6.49 Hz, 1H), 9.17 (s, 1H), 9.42 (s, 1H).

### EXAMPLE 56

### 4-Fluoro-3-[4-(1-isopropyl-1H-pyrrolo[2,3-c]pyridin-3-yl)-pyrimidin-2-ylamino]-benzonitrile [56] (Hereinafter Referred to as “Compound [56]”)

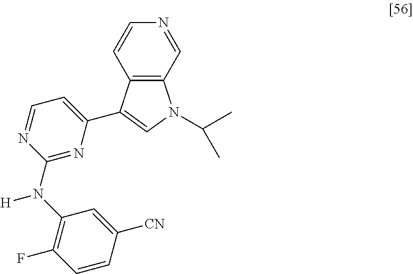

Compound [56] was prepared using a procedure similar to that of Example 44.

Data for Compound [54 LCMS m/e 372 (M+H); ¹H NMR (400 MHz, Methanol-d₄) δppm 1.71 (d, J=6.7 Hz, 6H), 5.06-5.17 (m, 1H), 7.40 (dd, J=10.8, 8.5 Hz, 1H), 7.47-7.53 (m, 2H), 8.35 (d, J=6.5 Hz, 1H), 8.54 (d, J=5.3 Hz, 1H), 8.77 (dd, J=7.5, 2.0 Hz, 1H), 9.02 (d, J=6.5 Hz, 1H), 9.16 (s, 1H), 9.40 (s, 1H).

### EXAMPLE 57

### 3-[4-(1-Isopropyl-1H-pyrrolo[2,3-c]pyridin-3-yl)-pyrimidin-2-ylamino]-4-methyl-benzonitrile [57] (Hereinafter Referred to as “Compound [57]”)

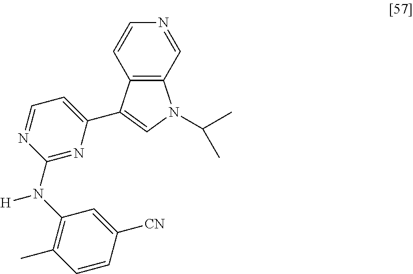

Compound [57] was prepared using a procedure similar to that of Example 44.

Data for Compound [57]: LCMS m/e 369 (M+H); ¹H NMR (400 MHz, Methanol-d₄) δppm 1.69 (s, 3H), 1.71 (s, 3H), 2.43 (s, 3H), 5.04-5.17 (m, 1H), 7.47-7.60 (m, 3H), 8.14 (d, J=1.37 Hz,1H), 8.28 (d, J=6.52 Hz, 1H), 8.42 (d, J=5.74 Hz, 1H), 8.66 (d, J=6.47 Hz, 1H), 9.22 (s, 1H), 9.41 (s, 1H).

### EXAMPLE 58

### 3-Fluoro-4-[4-(1-isopropyl-1H-pyrrolo[2,3-c]pyridin-3-yl)-pyrimidin-2-ylamino]-benzenesulfonamide [58] (Hereinafter Referred to as “Compound [58]”)

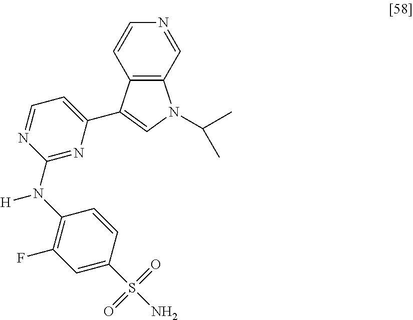

Compound [58] was prepared using a procedure similar to that of Example 44.

Data for Compound [58]: LCMS m/e 427 (M+H); ¹H NMR (400 MHz, Methanol-d₄) δ ppm 1.70 (s, 3H), 1.72 (s, 3H), 4.98-5.19 (m, 1H), 7.49 (d, J=5.37 Hz, 1H), 7.66-7.78 (m, 2H), 8.30 (d, J=6.54 Hz, 1H), 8.38 (t, J=8.30 Hz, 1H), 8.51 (d, J=5.37 Hz, 1H), 8.94 (d, J=6.47 Hz, 1H), 9.16 (s, 1H), 9.38 (s, 1H).

### EXAMPLE 59

### 3-[4-(1-Isopropyl-1H-pyrrolo[2,3-c]pyridin-3-yl)-pyrimidin-2-ylamino]Benzenesulfonamide [59] (Hereinafter Referred to as “Compound [59]”)

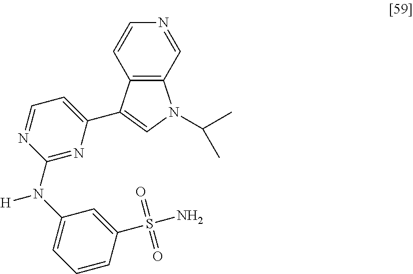

Compound [59] was prepared using a procedure similar to that of Example 44.

Data for Compound [59]: LCMS m/e 409 (M+H); ¹H NMR (400 MHz, Methanol-d₄) δppm 1.71 (s, 3H), 1.73 (s, 3H), 5.07-5.16 (m, 1H), 7.45 (d, J=5.37 Hz, 1H), 7.51 (t, J=7.88 Hz, 1H), 7.58-7.66 (m, 2H), 8.33 (d, J=6.54 Hz, 1H), 8.49 (d, J=5.37 Hz, 1H), 8.66-8.69 (m, 1H), 8.99 (d, J=6.47 Hz, 1H), 9.16 (s, 1H), 9.38 (s, 1H).

### EXAMPLE 60

### 3-Bromo-2-[4-(1-isopropyl-1H-pyrrolo[2,3-c]pyridin-3-yl)-pyrimidin-2-ylamino]-benzonitrile [60] (Hereinafter Referred to as “Compound [60]”)

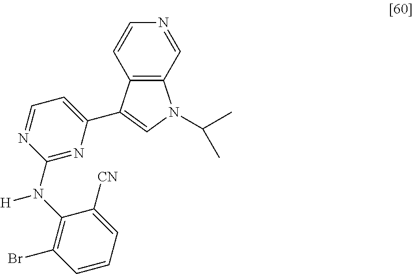

Compound [60] was prepared using a procedure similar to that of Example 44.

Data for Compound [60]: LCMS m/e 434 (M+H); ¹H NMR (400 MHz, Methanol-d₄) δppm 1.75 (s, 3H), 1.76 (s, 3H), 5.08-5.18 (m, 1H), 7.32 (t, J=7.82 Hz, 1H), 7.75 (d, J=7.61 Hz, 1H), 8.16(dd, J=7.61, 1.42 Hz, 1H), 8.24 (dd, J=8.02, 1.40 Hz, 1H), 8.49 (d, J=6.47 Hz, 1H), 9.12 (d, J=7.57 Hz, 1H), 9.38 (s, 1H), 9.46 (s, 1H), 9.56 (d, J=6.44 Hz, 1H).

### EXAMPLE 61

### (3,5-Dibromo-phenyl)-[4-(1-isopropyl-1H-pyrrolo[2,3-c]pyridin-3-yl)-pyrimidin-2-yl]-amine [61] (Hereinafter Referred to as “Compound [61]”)

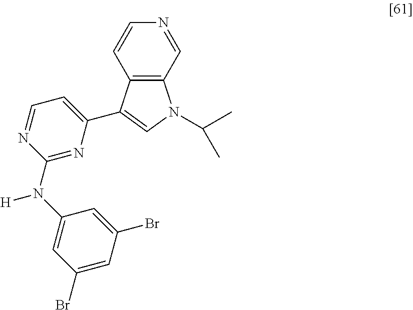

Compound [61] was prepared using a procedure similar to that of Example 44.

Data for Compound [61]: LCMS m/e 488 (M+H); ¹H NMR (400 MHz, Methanol-d₄) δ ppm 1.71 (s, 3H), 1.72 (s, 3H), 5.06-5.17 (m, 1H), 7.27 (t, J=1.64 Hz, 1H), 7.42 (d, J=5.34 Hz, 1H), 7.93 (s, 1H), 7.93 (s, 1H), 8.35 (d, J=6.52 Hz, 1H), 8.47 (d, J=5.34 Hz, 1H), 9.06 (d, J=6.49 Hz, 1H), 9.12 (s, 1H), 9.39 (s, 1H).

### EXAMPLE 62

### 3-[4-(1-Isopropyl-1H-pyrrolo[2,3-c]pyridin-3-yl)-pyrimidin-2-ylamino]-N-methyl-benzenesulfonamide [62] (Hereinafter Referred to as “Compound [62]”)

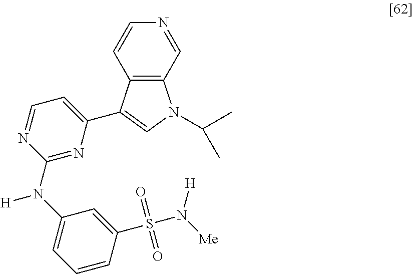

Compound [62] was prepared using a procedure similar to that of Example 44.

Data for Compound [62]: LCMS m/e 423 (M+1); ¹H NMR (400 MHz, Methanol-d₄) δppm 1.72 (s, 3H), 1.73 (s, 3H), 2.54 (s, 3H), 5.04-5.18 (m, 1H), 7.44 (d, J=5.32 Hz, 1H), 7.47-7.58 (m, 2H), 7.71 (d, J=7.74 Hz, 1H), 8.34 (d, J=6.52 Hz, 1H), 8.51 (d, J=5.30 Hz, 1H), 8.63 (s, 1H), 9.02 (d, J=6.49 Hz, 1H), 9.16 (s, 1H), 9.38 (s, 1H).

### EXAMPLE 63

### 4-[4-(1-Isopropyl-1H-pyrrolo[2,3-c]pyridin-3-yl)-pyrimidin-2-ylamino]-3-methyl-benzenesulfonamide [63] (Hereinafter Referred to as “Compound [63]”)

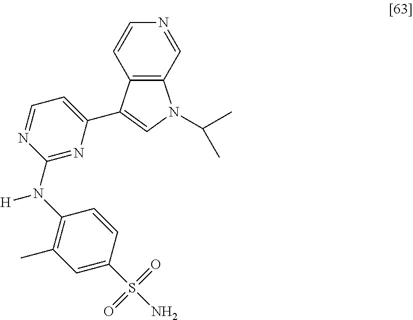

Compound [63] was prepared using a procedure similar to that of Example 44.

Data for Compound [63]: LCMS m/e 423 (M+H); 1H NMR (400 MHz, Methanol-d₄) δppm 1.68 (s, 3H), 1.70 (s, 3H), 2.39 (s, 3H), 5.03-5.18 (m, 1H), 7.45 (d, J=5.64 Hz, 1H), 7.77-7.85 (m, 2H), 7.89 (s, 1H), 8.19 (d, J=6.52 Hz, 1H), 8.41 (d, J=5.64 Hz, 1H), 8.45 (d, J=6.49 Hz, 1H), 9.18 (s, 1H), 9.37 (s, 1H).

### EXAMPLE 64

### 5-[4-(1-Isopropyl-1H-pyrrolo[2,3-c]pyridin-3-yl)-pyrimidin-2-ylamino]-2-methoxy-benzonitrile [64] (Hereinafter Referred to as “Compound [64]”)

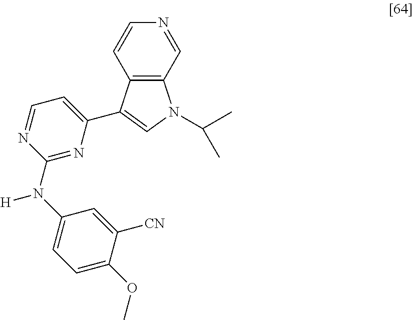

Compound [64] was prepared using a procedure similar to that of Example 44.

Data for Compound [64]: LCMS m/e 385 (M+H); ¹H NMR (400 MHz, Methanol-d₄) δppm 1.70 (s, 3H), 1.72 (s, 3H), 3.96 (s, 3H), 5.06-5.16 (m, 1H), 7.20 (d, J=9.13 Hz, 1H), 7.41 (d, J=5.49 Hz, 1H), 7.76 (dd, J=9.08, 2.71 Hz, 1H), 8.18 (d, J=2.71 Hz, 1H), 8.35 (d, J=6.52 Hz, 1H), 8.43 (d, J=5.49 Hz, 1H), 9.01 (d, J=6.49 Hz, 1H), 9.16 (s, 1H), 9.40 (s, 1H).

### EXAMPLE 65

### 3-[4-(1-Isopropyl-1H-pyrrolo[2,3-c]pyridin-3-yl)-pyrimidin-2-ylamino]-5-methoxy-benzonitrile [65] (Hereinafter Referred to as “Compound [65]”)

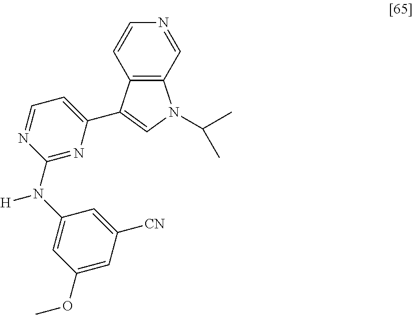

Compound [65] was prepared using a procedure similar to that of Example 44.

Data for Compound [65]: LCMS m/e 385 (M+H); ¹H NMR (400 MHz, Methanol-d₄) δ ppm 1.71 (s, 3H), 1.72 (s, 3H), 3.85 (s, 3H), 5.04-5.18 (m, 1H), 6.92 (dd, J=2.18, 1.31 Hz, 1H), 7.44 (d, J=5.37 Hz, 1H), 7.56 (t, J=2.12 Hz, 1H), 7.89 (t, J=1.54 Hz, 1H), 8.35 (d, J=6.52 Hz, 1H), 8.50 (d, J=5.37 Hz, 1H), 9.11 (d, J=6.49 Hz, 1H), 9.14 (s, 1H), 9.40 (s, 1H).

### EXAMPLE 66

### 3-[4-(1-Isopropyl-1H-pyrrolo[2,3-c]pyridine-3-yl)-pyrimidin-2-ylamino]-2-methyl-benzonitrile [66] (Hereinafter Referred to as “Compound [66]”)

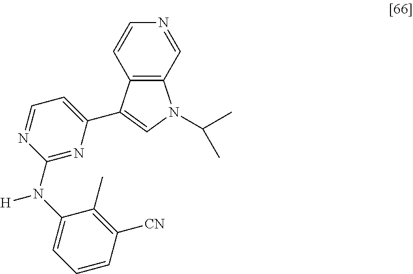

Compound [66] was prepared using a procedure similar to that of Example 44.

Data for Compound [66]: LCMS m/e 369 (M+H); 1H NMR (400 MHz, Methanol-d₄) δppm 1.69 (s, 3H), 1.70 (s, 3H), 2.50 (s, 3H), 5.05-5.16 (m, 1H), 7.46-7.52 (m, 2H), 7.70 (d, J=6.91 Hz,1H), 7.84 (d, J=7.27 Hz, 1H), 8.23 (d, J=6.52 Hz, 1H), 8.37 (d, J=5.83 Hz, 1H), 8.51 (d, J=6.44 Hz, 1H), 9.22 (s, 1H), 9.40 (s, 1H).

### EXAMPLE 67

### 5-[4-(1-Isopropyl-1H-pyrrolo[2,3-c]pyridin-3-yl)-pyrimidin-2-ylamino]-isoindole-1,3-dione [67] (Hereinafter Referred to as “Compound [67]”)

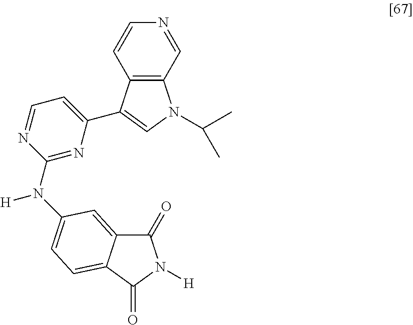

Compound [67] was prepared using a procedure similar to that of Example 44.

Data for Compound [67]: LCMS m/e 399 (M+H); 1H NMR (400 MHz, Methanol-d₄) δ ppm 1.72 (s, 3H), 1.73 (s, 3H), 5.06-5.18 (m, 1H), 7.50 (d, J=5.32 Hz, 1H), 7.75 (d, J=8.22 Hz, 1H), 7.89 (dd, J=8.24, 1.94 Hz, 1H), 8.36 (d, J=6.54 Hz, 1H), 8.57 (d, J=5.32 Hz, 1H), 8.63 (d, J=1.81 Hz, 1H), 9.12-9.19 (m, 2H), 9.40 (s, 1H).

### EXAMPLE 68

### 3-Bromo-4-[4-(1-isopropyl-1H-pyrrolo[2,3-c]pyridin-3-yl)-pyrimidin-2-ylamino]-benzonitrile [68] (Hereinafter Referred to as “Compound [68]”)

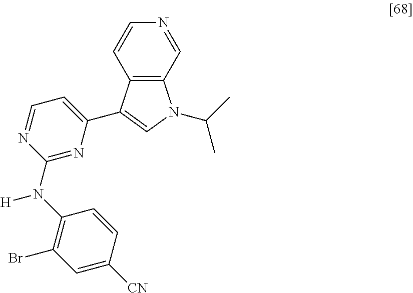

Compound [68] was prepared using a procedure similar to that of Example 44.

Data for Compound [68]: LCMS m/e 434 (M+H); 1H NMR (400 MHz, Methanol-d₄) δppm 1.70 (s, 3H), 1.72 (s, 3H), 5.07-5.17 (m, 1H), 7.56 (d, J=5.37 Hz, 1H), 7.75 (dd, J=8.64, 1.93 Hz, 1H), 8.07 (d, J=1.88 Hz, 1H), 8.37 (d, J=6.54 Hz, 1H), 8.54-8.60 (m, 2H), 8.94 (d, J=6.49 Hz, 1H), 9.18 (s, 1H), 9.41 (s, 1H).

### EXAMPLE 69

### 3-[4-(1-Isopropyl-1H-pyrrolo[2,3-c]pyridin-3-yl)-pyrimidin-2-ylamino]-N,N-dimethyl-benzenesulfonamide [69] (Hereinafter Referred to as “Compound [69]”)

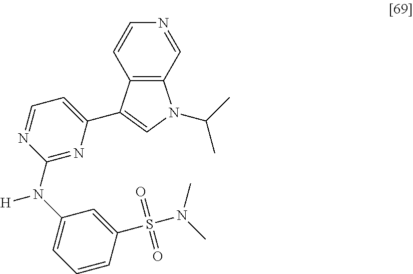

Compound [69] was prepared using a procedure similar to that of Example 44.

Data for Compound [69]: LCMS m/e 437 (M+H); 1H NMR (400 MHz, Methanol-d₄) δ ppm 1.71 (s, 3H), 1.73 (s, 3H), 2.69 (s, 6H), 5.06-5.15 (m, 1H), 7.39-7.45 (m, 2H), 7.55 (t, J=7.96 Hz,1H), 7.79 (dd, J=7.83, 1.61 Hz, 1H), 8.32 (d, J=6.52 Hz, 1H), 8.44 (t, J=1.85 Hz, 1H), 8.47 (d, J=5.44 Hz, 1H), 8.99 (d, J=6.47 Hz, 1H), 9.13 (s, 1H), 9.37 (s, 1H).

### EXAMPLE 70

### 3-Chloro-5-[4-(1-isopropyl-1H-pyrrolo[2,3-c]pyridin-3-yl)-pyrimidin-2-ylamino]-benzonitrile [70] (Hereinafter Referred to as “Compound [70]”)

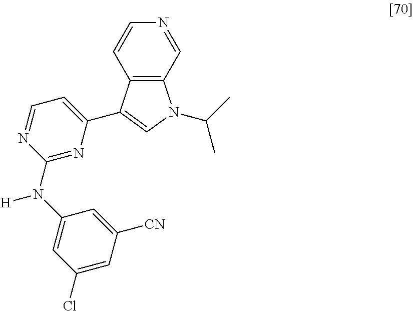

Compound [70] was prepared using a procedure similar to that of Example 44.

Data for Compound [70]: LCMS m/e 390 (M+1); ¹H NMR (400 MHz, Methanol-d₄) δ ppm 1.71 (s, 3H), 1.73 (s, 3H), 5.07-5.17 (m, 1H), 7.35 (m, 1H), 7.48 (d, J=5.32 Hz, 1H), 8.15 (t, J=1.87 Hz, 1H), 8.18 (m, 1H), 8.38 (d, J=6.49 Hz, 1H), 8.55 (d, J=5.32 Hz, 1H), 9.12 (d, J=6.49 Hz, 1H), 9.15 (s, 1H), 9.41 (s, 1H).

### Example 71

### 3-[4-(1-Isopropyl-1H-pyrrolo[2,3-c]pyridin-3-yl)-pyrimidin-2-ylamino]-4-methoxy-N-methyl-benzamide [71] (Hereinafter Referred to as “Compound [71]”)

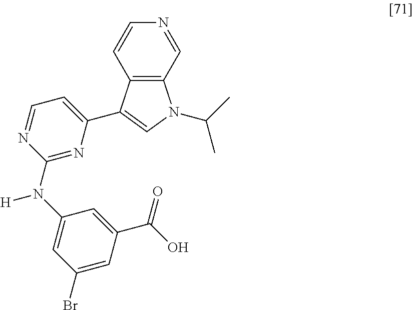

Compound [71] was prepared using a procedure similar to that of Example 44.

Data for Compound [71]: LCMS m/e 417 (M+1); ¹H NMR (400 MHz, Methanol-d₄) δ ppm 1.71 (s, 3H), 1.72 (s, 3H), 2.90 (s, 3H), 3.98 (s, 3H), 5.05-5.16 (m, 1H), 7.18 (d, J=8.61 Hz, 1H), 7.51 (d, J=5.83 Hz, 1H), 7.69 (dd, J=8.57, 2.22 Hz, 1H), 8.26 (d, J=6.49 Hz, 1H), 8.41 (d, J=5.81 Hz, 1H), 8.58 (d, J=2.17 Hz, 1H), 8.86 (d, J=6.49 Hz, 1H), 9.24 (s, 1H), 9.41 (s, 1H).

### EXAMPLE 72

### 3-[4-(1-Isopropyl-1H-pyrrolo[2,3-c]pyridin-3-yl)-pyrimidin-2-ylamino[-4-methyl-benzoic acid methyl ester [72] (Hereinafter Referred to as “Compound [72]”)

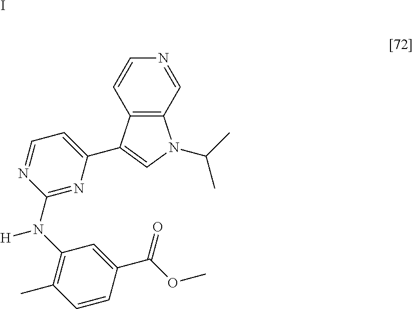

Compound [72] was prepared using a procedure similar to that of Example 44.

Data for Compound [72]: LCMS m/e 402 (M+H); ¹H NMR (400 MHz, Methanol-d₄) δ ppm 1.69 (s, 3H), 1.71 (s, 3H), 2.40 (s, 3H), 3.87 (s, 3H), 5.02-5.15 (m, 1H), 7.46-7.52 (m, 2H). 7.91 (dd, J=7.98, 1.66 Hz, 1H) 8.15 (d, J=6.49 Hz, 1H) 8.28 (d, J=1.64 Hz, 1H) 8.37 (d, J=5.83 Hz, 1H) 8.59 (d, J=6.42 Hz, 1H) 9.22 (s, 1H) 9.38 (s, 1H).

### EXAMPLE 73

### 3-Bromo-5-[4-(1-isopropyl-1H-pyrrolo[2,3-c]pyridine-3-yl)-pyrimidin-2-ylamino]-benzoic acid [73] (Hereinafter Referred to as “Compound [73]”)

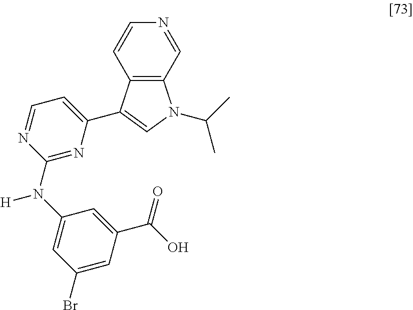

Compound [73] was prepared using a procedure similar to that of Example 44.

Data for Compound [73]: LCMS m/e 453 (M+H); ¹H NMR (400 MHz, Methanol-d₄) δ ppm 1.71 (s, 3H), 1.73 (s, 3H), 5.06-5.17 (m, 1H), 7.44 (d, J=5.32 Hz, 1H), 7.77 (t, J=1.55 Hz, 1H), 8.26 (dd, 1H), 8.33 (d, J=6.52 Hz, 1H), 8.36-8.40 (m, 1H), 8.51 (d, J=5.30 Hz, 1H), 9.08 (d, J=6.47 Hz, 1H), 9.15 (s, 1H), 9.39 (s, 1H).

### EXAMPLE 74

### 3-[4-(1-Isopropyl-1H-pyrrolo[2,3-c]pyridin-3-yl)-pyrimidin-2-ylamino]-benzoic acid [74] (Hereinafter Referred to as “Compound [74]”)

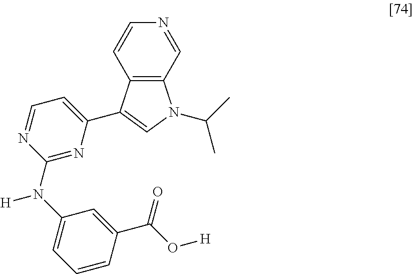

Compound [74] was prepared using a procedure similar to that of Example 44.

Data for Compound [74]: LCMS m/e 374 (M+H); ¹H NMR (400 MHz, Methanol-d₄) δppm 1.70 (s, 3H), 1.72 (s, 3H), 5.05-5.16 (m, 1H), 7.42-7.51 (m, 2H), 7.75 (d, J=2.15 Hz, 2H), 8.28 (d, J=6.64 Hz, 1H), 8.54 (t, J=1.56 Hz, 1H), 9.00 (d, J=6.44 Hz, 1H), 9.18 (s, 1H), 9.38 (s, 1H).

### EXAMPLE 75

### 2-Fluoro-3-[4-(1-isopropyl-1H-pyrrolo[2,3-c]pyridin-3-yl)-pyrimidin-2-ylamino]-benzonitrile [75] (Hereinafter Referred to as “Compound [75]”)

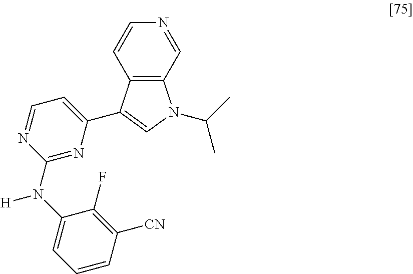

Compound [75] was prepared using a procedure similar to that of Example 44.

Data for Compound [75]: LCMS m/e 373 (M+H); 1H NMR (400 MHz, Methanol-d₄) δ ppm 1.63 (s, 3H), 1.65 (s, 3H), 4.93-5.03 (m, 1H), 7.32-7.40 (m, 2H), 7.44-7.51 (m, 1H), 8.18 (d, J=5.64 Hz, 1H), 8.30-8.42 (m, 3H), 8.52 (s, 1H), 8.89 (s, 1H).

### EXAMPLE 76

### 3-Fluoro-5-[4-(1-isopropyl-1H-pyrrolo[2,3-c]pyridin-3-yl)-pyrimidin-2-ylamino]-benzonitrile [76] (Hereinafter Referred to as “Compound [76]”)

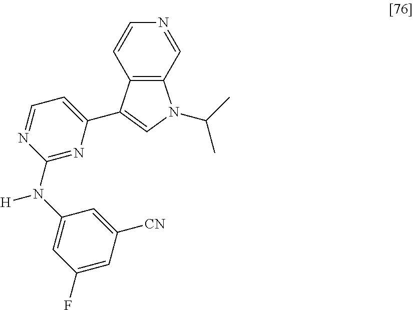

Compound [76] was prepared using a procedure similar to that of Example 44.

Data for Compound [76]: LCMS m/e 373 (M+H); ¹H NMR (400 MHz, Methanol-d₄) δ ppm 1.65 (s, 3H), 1.66 (s, 3H), 4.95-5.03 (m, 1H), 7.06-7.11 (m, 1H), 7.36 (d, J=5.39 Hz, 1H), 7.93-7.99 (m, 1H), 8.08-8.11 (m, 1H), 8.26 (d, J=5.61 Hz, 1H), 8.42 (d, J=5.39 Hz, 1H), 8.52-8.54 (m, 2H), 8.90 (s, 1H).

### EXAMPLE 77

### 2-Fluoro-5-[4-(1-isopropyl-1H-pyrrolo[2,3-c]pyridine-3-yl)-pyrimidin-2-ylamino]-benzonitrile [77] (Hereinafter Referred to as “Compound [77]”)

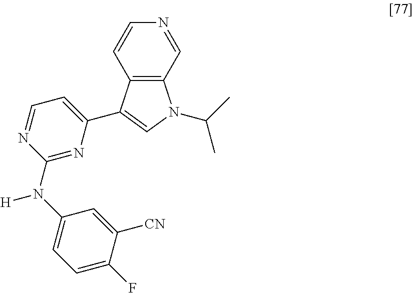

Compound [77] was prepared using a procedure similar to that of Example 44.

Data for Compound [77]: LCMS m/e 373 (M+H); 1H NMR (400 MHz, Methanol-d₄) δ ppm 1.71 (s, 3H), 1.72 (s, 3H), 5.06-5.17 (m, 1H), 7.33 (t, J=8.98 Hz, 1H), 7.44 (d, J=5.32 Hz, 1H), 7.86-7.92 (m, 1H), 8.34-8.42 (m, 2H), 8.51 (d, J=5.32 Hz, 1H), 9.09 (d, J=6.47 Hz, 1H), 9.14 (s, 1H), 9.40 (s, 1H).

### EXAMPLE 78

### 3-[4-(1-Isopropyl-1H-pyrrolo[2,3-c]pyridine-3-yl)-pyrimidin-2-ylamino]-4-methyl-benzoic acid [78] (Hereinafter Referred to as “Compound [78]”)

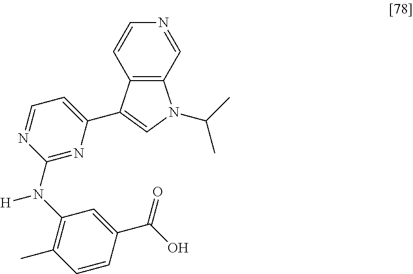

Compound [78] was prepared using a procedure similar to that of Example 44.

Data for Compound [78]: LCMS m/e 388 (M+H); ¹H NMR (400 MHz, Methanol-d₄) δppm 1.69 (s, 3H), 1.70 (s, 3H), 2.41 (s, 3H), 5.05-5.15 (m, 1H), 7.48-7.50 (m, 1H) 7.51 (s, 1H) 7.94 (dd, J=7.92, 1.67 Hz, 1H) 8.16 (d, J=6.49 Hz, 1H) 8.27 (d, J=1.56 Hz, 1H) 8.36 (d, J=5.95 Hz, 1H) 8.56 (d, J=6.54 Hz, 1H) 9.24 (s, 1H) 9.39 (s,1H).

### EXAMPLE 79

### 3-Bromo-5-[4-(1-isopropyl-1H-pyrrolo[2,3-c]pyridin-3-yl)-pyrimidin-2-ylamino]-benzoic acid methyl ester [79] (Hereinafter Referred to as “Compound [79]”)

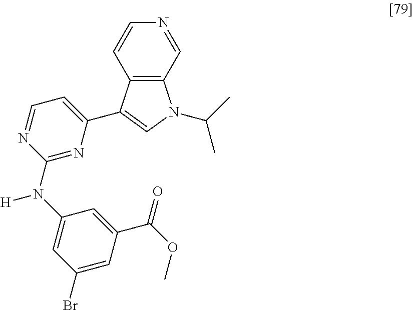

Compound [79] was prepared using a procedure similar to that of Example 44.

Data for Compound [79]: LCMS m/e 467 (M+H); ¹H NMR (400 MHz, Methanol-d₄) δ ppm 1.71 (s, 3H), 1.73 (s, 3H), 3.91 (s, 3H), 5.07-5.17 (m, 1H), 7.45 (d, J=5.30 Hz, 1H), 7.78 (t, J=1.59 Hz, 1H), 8.31 (t, J=1.93 Hz, 1H), 8.34 (d, J=6.52 Hz, 1H), 8.38-8.41 (m, 1H), 8.53 (d, J=5.30 Hz, 1H), 9.09 (d, J=6.47 Hz, 1H), 9.16 (s, 1H), 9.40 (s, 1H).

### EXAMPLE 80

### 5-[4-(1-Isopropyl-1H-pyrrolo[2,3-c]pyridin-3-yl)-pyrimidin-2-ylamino]-2-methyl-benzenesulfonamide [80] (Hereinafter Referred to as “Compound [80]”)

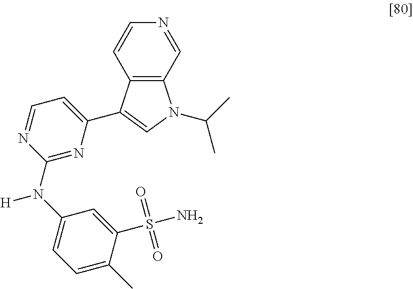

Compound [80] was prepared using a procedure similar to that of Example 44.

Data for Compound [80]: LCMS m/e 423 (M+H); ¹H NMR (400 MHz, Methanol-d₄) δppm 1.72 (s, 3H), 1.73 (s, 3H), 2.67 (s, 3H), 5.06-5.16 (m, 1H), 7.34 (d, J=8.25 Hz, 1H), 7.42 (d, J=5.42 Hz, 1H), 7.55 (dd, J=8.11, 2.31 Hz, 1H), 8.32 (d, J=6.49 Hz, 1H), 8.46 (d, J=5.37 Hz, 1H), 8.67 (d, J=2.27 Hz, 1H), 8.94 (d, J=6.54 Hz, 1H), 9.19 (s, 1H), 9.37 (s, 1H).

### EXAMPLE 81

### N,N-Diethyl-4-[4-(1-isopropyl-1H-pyrrolo[2,3-c]pyridin-3-yl)-pyrimidin-2-ylamino]-3-methoxy-benzamide [81] (Hereinafter Referred to as “Compound [81]”)

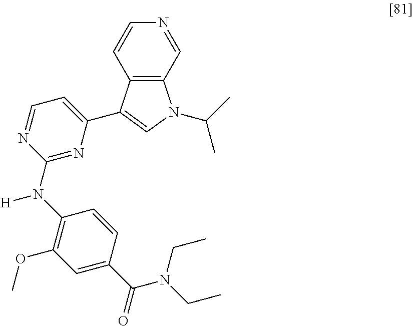

Compound [81] was prepared using a procedure similar to that of Example 44.

Data for Compound [81]: LCMS m/e 459 (M+H); ¹H NMR (400 MHz, Methanol-d₄) δ ppm 1.24 (br. s., 6H), 1.70 (s, 3H), 1.72 (s, 3H), 3.38-3.49 (m, 2H), 3.50-3.63 (m, 2H), 3.94 (s, 3H),5.05-5.16 (m, 1H), 7.07 (dd, J=8.11, 1.72 Hz, 1H), 7.13 (d, J=1.66 Hz, 1H), 7.49 (d, J=5.83 Hz, 1H), 8.10 (d, J=8.10 Hz, 1H), 8.37 (d, J=6.56 Hz,1H), 8.41 (d, J=5.81 Hz, 1H), 8.82 (d, J=6.49 Hz, 1H), 9.21 (s, 1H), 9.40 (s, 1H).

### EXAMPLE 82

### [4-(1-Isopropyl-1H-pyrrolo[2,3-c]pyridin-3-yl)-pyrimidin-2-yl]-[3-(piperazine-1-sulfonyl)-phenyl]-amine [82] (Hereinafter Referred to as “Compound [82]”)

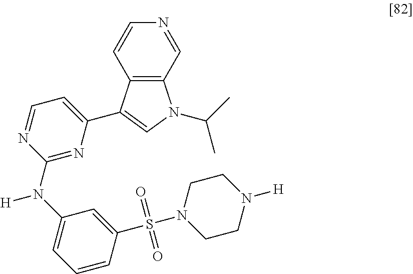

Compound [82] was prepared using a procedure similar to that of Example 44.

Data for Compound [82]: LCMS m/e 478 (M+H); ¹H NMR (400 MHz, DMSO-d₆) δ ppm 1.60 (s, 3H), 1.62 (s, 3H), 3.13 (br. s., 4H), 3.20 (br. s., 4H), 5.06-5.23 (m, 1H), 7.35 (d, J=7.83 Hz, 1H), 7.55 (d, J=5.32 Hz, 1H), 7.65 (t, J=8.02 Hz, 1H), 8.13 (d, J=8.27 Hz, 1H), 8.33 (s, 1H), 8.47 (d, J=6.37 Hz, 1H), 8.57 (d, J=5.32 Hz, 1H), 8.63 (br.s., 2H), 9.02 (d, J=6.00 Hz, 1H), 9.27 (s, 1H), 9.53 (s, 1H), 10.11 (s, 1H).

### EXAMPLE 83

### N-(2-Hydroxy-ethyl)-4-[4-(1-isopropyl-1H-pyrrolo[2,3-c]pyridin-3-yl)-pyrimidin-2-ylamino]-benzamide [83] (Hereinafter Referred to as “Compound [83]”)

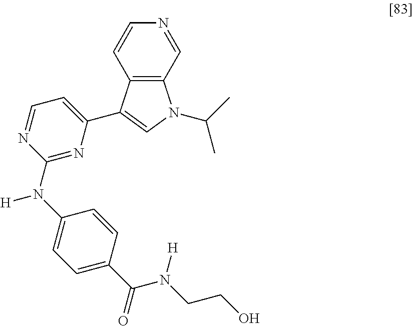

Compound [83] was prepared using a procedure similar to that of Example 44.

Data for Compound [83]: LCMS m/e 417 (M+H); ¹H NMR (400 MHz, Methanol-d₄) δ ppm 1.70 (s, 3H), 1.72 (s, 3H), 3.53 (t, J=5.81 Hz, 2H), 3.73 (t, J=5.81 Hz, 2H), 5.02-5.15 (m, 1H), 7.43 (d, J=5.54 Hz, 1H), 7.72-7.87 (m, 4H), 8.35 (d, J=6.52 Hz, 1H), 8.44 (d, J=5.52 Hz, 1H), 9.06 (d, J=6.49 Hz, 1H), 9.14 (s, 1H), 9.37 (s, 1H).

### EXAMPLE 84

### 3-[4-(1-Isopropyl-1H-pyrrolo[2,3-c]pyridin-3-yl)-pyrimidin-2-ylamino]-4,5-dimethoxy-benzonitrile [84] (Hereinafter Referred to as “Compound [84]”)

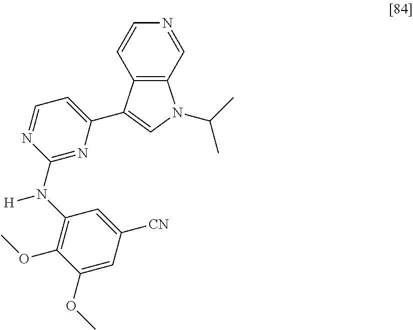

Compound [84] was prepared using a procedure similar to that of Example 44.

Data for Compound [84]: LCMS m/e 415 (M+H); ¹H NMR (400 MHz, Methanol-d₄) δppm 1.71 (s, 3H), 1.73 (s, 3H), 3.93 (s, 3H), 3.98 (s, 3H), 5.07-5.17 (m, 1H), 7.14 (d, J=1.83 Hz, 1H), 7.49 (d, J=5.49 Hz, 1H), 8.38 (d, J=6.52 Hz, 1H), 8.45 (d, J=1.83 Hz, 1H), 8.50 (d, J=5.44 Hz, 1H), 8.94 (d, J=6.49 Hz, 1H), 9.16 (s, 1H), 9.42 (s, 1H).

### EXAMPLE 85

### 3-[4-(1-Isopropyl-1H-pyrrolo[2,3-c]pyridin-3-yl)-pyrimidin-2-ylamino]-4-methyl-benzamide [85] (Hereinafter Referred to as “Compound [85]”)

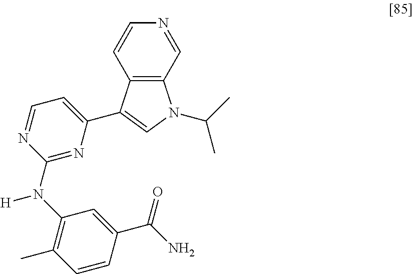

Compound [85] was prepared using a procedure similar to that of Example 44.

Data for Compound [85]: LCMS m/e 387 (M+H); 1H NMR (400 MHz, Methanol-d₄) δppm 1.68 (s, 3H), 1.70 (s, 3H), 2.39 (s, 3H), 5.03-5.14 (m, 1H), 7.48 (s, 1H), 7.49 (s, 1H), 7.79 (dd, J=7.94, 1.82 Hz, 1H), 8.13 (d, J=1.73 Hz, 1H), 8.16 (d, J=6.49 Hz, 1H), 8.36 (d, J=5.83 Hz, 1H), 8.55 (d, J=6.37 Hz, 1H), 9.23 (s, 1H), 9.38 (s, 1H).

### EXAMPLE 86

### 4-[4-(1-Isopropyl-1H-pyrrolo[2,3-c]pyridine-3-yl)-pyrimidin-2-ylamino]-3-methyl-benzamide [86] (Hereinafter Referred to as “Compound [86]”)

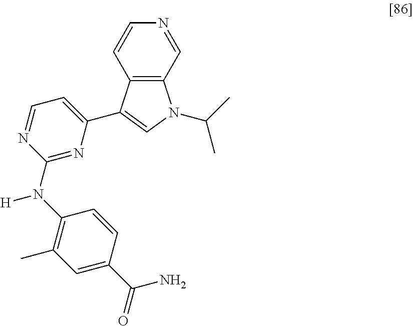

Compound [86] was prepared using a procedure similar to that of Example 44.

Data for Compound [86]: LCMS m/e 387 (M+H); ¹H NMR (400 MHz, Methanol-d₄) δ ppm 1.69 (s, 3H), 1.71 (s, 3H), 2.39 (s, 3H), 5.05-5.15 (m, 1H), 7.52 (d, J=5.98 Hz, 1H), 7.72 (d, J=8.30 Hz, 1H), 7.80-7.86 (m, 1H), 7.90 (d, J=1.61 Hz, 1H), 8.22 (d, J=6.49 Hz, 1H), 8.38 (d, J=5.95 Hz, 1H), 8.55 (d, J=6.49 Hz, 1H), 9.25 (s, 1H), 9.40 (s, 1H).

### EXAMPLE 87

### 2-Fluoro-5-[4-(1-isopropyl-1H-pyrrolo[2,3-c]pyridin-3-yl)-pyrimidin-2-ylamino]-benzoic acid [87] (Hereinafter Referred to as “Compound [87]”)

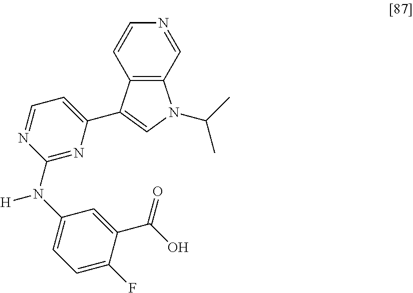

Compound [87] was prepared using a procedure similar to that of Example 44.

Data for Compound [87]: LCMS m/e 392 (M+H); 1H NMR (400 MHz, Methanol-d₄) δppm 1.70 (s, 3H), 1.72 (s, 3H), 5.06-5.15 (m, 1H), 7.20 (dd, J=10.27, 8.98 Hz, 1H), 7.39 (d, J=5.32 Hz, 1H), 7.74-7.80 (m, 1H), 8.29 (d, J=6.61 Hz, 1H), 8.44 (dd, J=6.52, 2.98 Hz, 1H), 8.46 (d, J=5.32 Hz, 1H), 9.03 (d, J=6.42 Hz, 1H), 9.14 (s, 1H), 9.38 (s, 1H).

### EXAMPLE 88

### 3-Bromo-5-[4-(1-isopropyl-1H-pyrrolo[2,3-c]pyridin-3-yl)-pyrimidin-2-ylamino]-benzamide [88] (Hereinafter Referred to as “Compound [88]”)

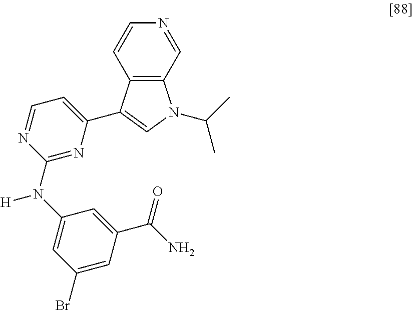

Compound [88] was prepared using a procedure similar to that of Example 44.

Data for Compound [88]: LCMS m/e 452 (M+H); ¹H NMR (400 MHz, Methanol-d₄) δ ppm 1.71 (s, 3H), 1.72 (s, 3H), 5.06-5.15 (m, 1H), 7.44 (d, J=5.44 Hz, 1H), 7.66 (t, J=1.57 Hz, 1H), 8.15 (t, J=1.72 Hz, 1H), 8.17 (t, J=1.83 Hz, 1H), 8.31 (d, J=6.54 Hz, 1H), 8.47 (d, J=5.42 Hz, 1H), 9.03 (d, J=6.52 Hz, 1H), 9.15 (s, 1H), 9.38 (s, 1H).

### EXAMPLE 89

### 3-[4-(1-Isopropyl-1H-pyrrolo[2,3-c]pyridin-3-yl)-pyrimidin-2-ylamino]-5-trifluoromethyl-benzoic acid [89] (Hereinafter Referred to as “Compound [89]”)

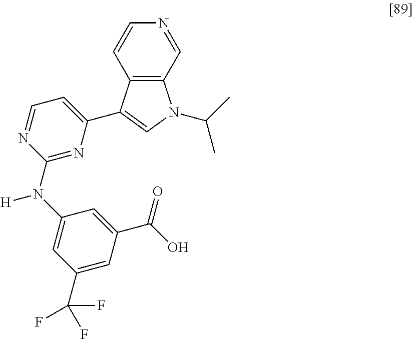

Compound [89] was prepared using a procedure similar to that of Example 44.

Data for Compound [89]: LCMS m/e 442 (M+H); ¹H NMR (400 MHz, Methanol-d₄) δppm 1.71 (s, 3H), 1.73 (s, 3H), 5.07-5.17 (m, 1H), 7.46 (d, J=5.30 Hz, 1H), 7.88 (s, 1H), 8.31 (d, J=6.47 Hz, 1H), 8.37 (br. s., 1H), 8.54 (d, J=5.27 Hz, 1H), 8.71 (s, 1H), 9.06 (d, J=6.49 Hz, 1H), 9.14 (s, 1H), 9.40 (s, 1H).

### EXAMPLE 90

### 3-Chloro-5-[4-(1-isopropyl-1H-pyrrolo[2,3-c]pyridin-3-yl)-pyrimidin-2-ylamino]-benzoic acid [90] (Hereinafter Referred to as “Compound [90]”)

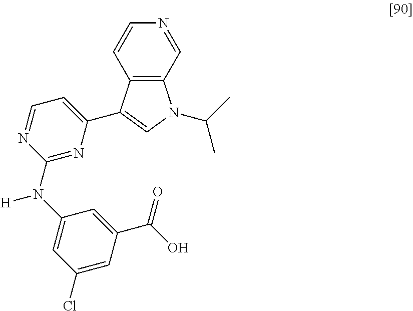

Compound [90] was prepared using a procedure similar to that of Example 44.

Data for Compound [90]: LCMS m/e 408 (M+H); ¹H NMR (400 MHz, Methanol-d₄) δppm 1.71 (s, 3H), 1.73 (s, 3H), 5.06-5.17 (m, 1H), 7.45 (d, J=5.34 Hz, 1H), 7.62 (t, J=1.62 Hz, 1H), 8.11 (t, J=1.99 Hz, 1H), 8.32 (d, J=6.52 Hz, 1H), 8.35 (t, J=1.67 Hz, 1H), 8.51 (d, J=5.32 Hz, 1H), 9.09 (d, J=6.49 Hz, 1H), 9.15 (s, 1H), 9.39 (s, 1H).

### EXAMPLE 91

### {3-[4-(1-Isopropyl-1H-pyrrolo[2,3-c]pyridin-3-yl)-pyrimidin-2-ylamino]-phenyl}-acetic acid [91] (Hereinafter Referred to as “Compound [91]”)

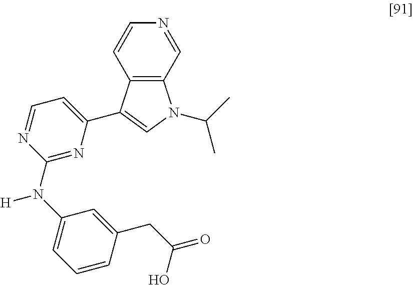

Compound [91] was prepared using a procedure similar to that of Example 44.

Data for Compound [91]: LCMS m/e 388 (M+H); ¹H NMR (400 MHz, Methanol-d₄) δppm 1.70 (s, 3H), 1.72 (s, 3H), 3.64 (s, 2H), 5.05-5.16 (m, 1H), 7.10 (d, J=7.54 Hz, 1H), 7.36 (t, J=7.83 Hz, 1H), 7.41 (d, J=5.64 Hz, 1H), 7.51 (dd, J=7.63, 1.57 Hz, 1H), 7.66 (m, 1H), 8.31 (d, J=6.52 Hz, 1H), 8.40 (d, J=5.64 Hz, 1H), 8.97 (d, J=6.44 Hz, 1H), 9.18 (s, 1H), 9.39 (s, 1H).

### EXAMPLE 92

### 2-{3-[4-(1-Isopropyl-1H-pyrrolo[2,3-c]pyridin-3-yl)-pyrimidin-2-ylamino]-phenyl}-acetamide [92] (Hereinafter Referred to as “Compound [92]”)

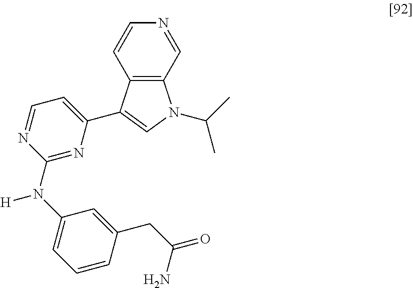

Compound [92] was prepared using a procedure similar to that of Example 44.

Data for Compound [92]: LCMS m/e 387 (M+H); ¹H NMR (400 MHz, Methanol-d₄) δ ppm 1.70 (s, 3H), 1.72 (s, 3H), 3.54 (s, 2H), 5.04-5.15 (m, 1H), 7.07 (d, J=7.81 Hz, 1H), 7.30-7.41 (m,2H), 7.52 (d, J=8.08 Hz, 1H), 7.68 (s, 1H), 8.32 (d, J=6.59 Hz, 1H), 8.41 (d, J=5.47 Hz, 1H), 9.03 (d, J=6.61 Hz, 1H), 9.15 (s, 1H), 9.37 (s, 1H).

### EXAMPLE 93

### 4-Benzyloxy-3-[4-(1-isopropyl-1H-pyrrolo[2,3-c]pyridin-3-yl)-pyrimidin-2-ylamino]-benzonitrile [93] (Hereinafter Referred to as “Compound [93]”)

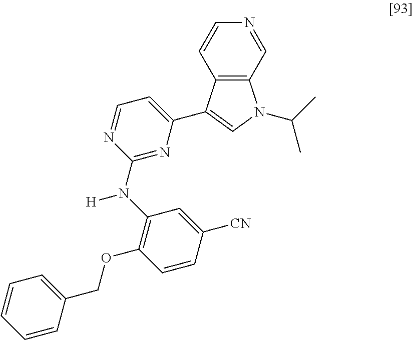

Compound [93] was prepared using a procedure similar to that of Example 44.

Data for Compound [93]: LCMS m/e 461 (M+H); ¹H NMR (400 MHz, Methanol-d₄) δppm 1.71 (s, 3H), 1.72 (s, 3H), 5.06-5.17 (m, 1H), 5.32 (s, 2H), 7.21-7.51 (m, 8H), 8.32 (d, J=6.49 Hz,1H), 8.49 (d, J=5.37 Hz, 1H), 8.75 (d, J=2.00 Hz, 1H), 8.86 (d, J=6.47 Hz, 1H), 9.15 (s, 1H), 9.41 (s, 1H).

### EXAMPLE 94

### 4-Benzyloxy-3-[4-(1-isopropyl-1H-pyrrolo[2,3-c]pyridin-3-yl)-pyrimidin-2-ylamino]-benzamide [94] (Hereinafter Referred to as “Compound [94]”)

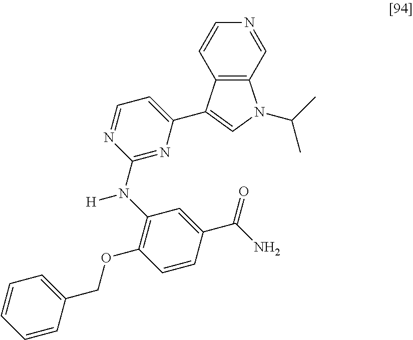

Compound [94] was prepared using a procedure similar to that of Example 44.

Data for Compound [94]: LCMS m/e 479 (M+H); ¹H NMR (400 MHz, DMSO-d₆) δ ppm 1.61 (s, 3H), 1.63 (s, 3H), 5.08-5.20 (m, 1H), 5.25 (s, 2H), 7.19-7.34 (m, 4H), 7.39-7.45 (m, 2H), 7.49 (d, J=5.37 Hz, 1H), 7.71 (dd, J=8.55, 2.01 Hz, 1H), 7.89 (br. s., 1H), 8.26 (d, J=6.42 Hz, 1H), 8.47 (d, J=2.07 Hz, 1H), 8.50 (d, J=5.34 Hz, 1H), 8.69 (br. s., 1H), 8.72 (d, J=6.42 Hz, 1H), 9.34 (s, 1H), 9.56 (s, 1H).

### EXAMPLE 95

### 3-[4-(1-Isopropyl-1H-pyrrolo[2,3-c]pyridin-3-yl)-pyrimidin-2-ylamino]-5-nitro-benzamide [95] (Hereinafter Referred to as “Compound [95]”)

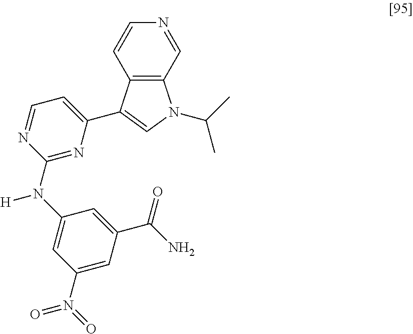

Compound [95] was prepared using a procedure similar to that of Example 44.

Data for Compound [95]: LCMS m/e 418 (M+H); ¹H NMR (400 MHz, Methanol-d₄) δ ppm 1.72 (s, 3H), 1.74 (s, 3H), 5.07-5.19 (m, 1H), 7.49 (d, J=5.32 Hz, 1H), 8.30-8.35 (m, 2H), 8.53 (t, J=1.56 Hz, 1H), 8.56 (d, J=5.27 Hz, 1H), 9.04-9.08 (m, 2H), 9.17 (s, 1H), 9.40 (s, 1H).

### EXAMPLE 96

### 1-Amino-3-[4-(1-Isopropyl-1H-pyrrolo[2,3-c]pyridin-3-yl)-pyrimidin-2-ylamino]-4-trifluoromethoxy-benzene [96] (Hereinafter Referred to as “Compound [96]”)

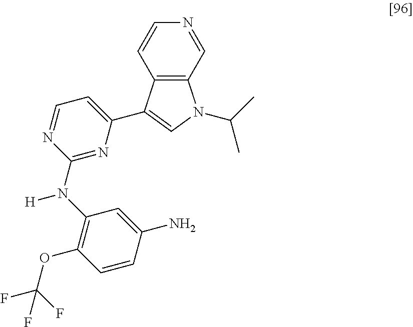

Compound [96] was prepared using a procedure similar to that of Example 44.

Data for Compound [96]: LCMS m/e 429 (M+H); ¹H NMR (400 MHz, Methanol-d₄) δppm 1.70 (s, 3H), 1.72 (s, 3H), 5.07-5.16 (m, 1H), 7.00 (dd, J=8.79, 2.71 Hz, 1H), 7.41 (dd, J=8.79, 1.27 Hz, 1H), 7.54 (d, J=5.66 Hz, 1H), 8.04 (d, J=2.59 Hz, 1H), 8.35 (d, J=6.49 Hz, 1H), 8.46 (d, J=5.64 Hz, 1H), 8.96 (d, J=6.49 Hz, 1H), 9.21 (s, 1H), 9.42 (s, 1H).

### EXAMPLE 97

### (3-Bromo-5-trifluoromethoxy-phenyl)-[4-(1-isopropyl-1H-pyrrolo[2,3-c]pyridin-3-yl)-pyrimidin-2-yl]-amine [97] (Hereinafter Referred to as “Compound [97]”)

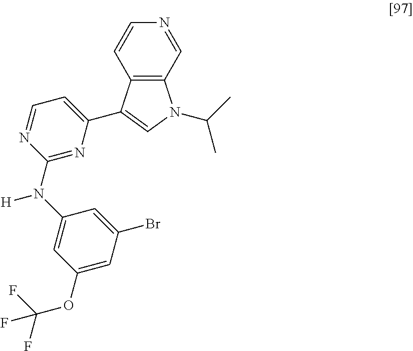

Compound [97] was prepared using a procedure similar to that of Example 44.

Data for Compound [97]: LCMS m/e 493 (M+H); ¹H NMR (400 MHz, Methanol-d₄) δppm 1.70 (s, 3H), 1.72 (s, 3H), 5.06-5.16 (m, 1H), 7.05 (s, 1H), 7.44 (d, J=5.34 Hz, 1H), 7.72 (s, 1H), 8.07 (t, J=1.74 Hz, 1H), 8.35 (d, J=6.52 Hz, 1H), 8.50 (d, J=5.34 Hz, 1H), 9.11 (d, J=6.54 Hz, 1H), 9.13 (s, 1H), 9.40 (s, 1H).

### EXAMPLE 98

### (3-Bromo-5-methyl-phenyl)-[4-(1-isopropyl-1H-pyrrolo[2,3-c]pyridin-3-yl)-pyrimidin-2-yl]-amine [98] (Hereinafter Referred to as “Compound [98]”)

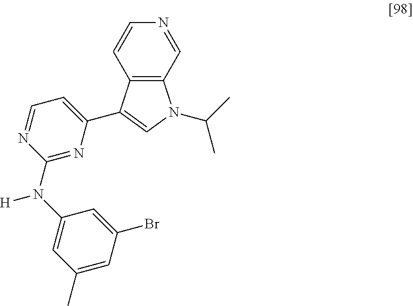

Compound [98] was prepared using a procedure similar to that of Example 44.

Data for Compound [98]: LCMS m/e 423 (M+H); ¹H NMR (400 MHz, Methanol-d₄) δppm 1.70 (s, 3H), 1.72 (s, 3H), 2.33 (s, 3H), 5.05-5.16 (m, 1H), 7.12 (br. s., 1H), 7.30 (br. s., 1H), 7.46 (d, J=5.76 Hz, 1H), 7.83 (br. s., 1H), 8.34 (d, J=6.49 Hz, 1H), 8.39 (d, J=5.74 Hz, 1H), 8.97 (d, J=6.47 Hz, 1H), 9.19 (s, 1H), 9.41 (s, 1H).

### EXAMPLE 99

### (3-Bromo-5-tert-butyl-phenyl)-[4-(1-isopropyl-1H-pyrrolo[2,3-c]pyridin-3-yl)-pyrimidin-2-yl]-amine [99] (Hereinafter Referred to as “Compound [99]”)

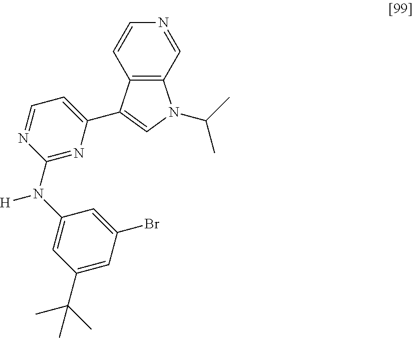

Compound [99] was prepared using a procedure similar to that of Example 44.

Data for Compound [99]: LCMS m/e 465 (M+H); ¹H NMR (400 MHz, Methanol-d₄) δppm 1.33 (s, 9H), 1.70 (s, 3H), 1.72 (s, 3H), 5.06-5.17 (m, 1H), 7.26 (t, J=1.62 Hz, 1H), 7.42 (d, J=5.47 Hz, 1H), 7.47 (t, J=1.65 Hz, 1H), 8.07 (t, J=1.78 Hz, 1H), 8.34 (d, J=6.52 Hz, 1H), 8.46 (d, J=5.47 Hz, 1H), 9.07 (d, J=6.47 Hz, 1H), 9.16 (s, 1H), 9.40 (s, 1H).

### EXAMPLE 100

### 3-[4-(1-Isopropyl-1H-pyrrolo[2,3-c]pyridin-3-yl)-pyrimidin-2-ylamino]-5-methyl-benzonitrile [100] (Hereinafter Referred to as “Compound [100]”)

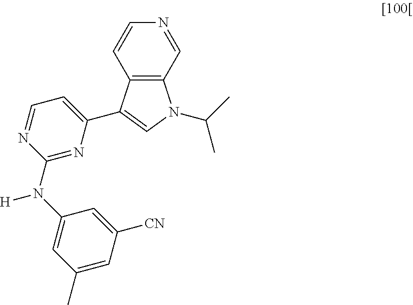

Compound [100] was prepared using a procedure similar to that of Example 44.

Data for Compound [100]: LCMS m/e 369 (M+H); ¹H NMR (400 MHz, Methanol-d₄) δppm 1.71 (s, 3H), 1.72 (s, 3H), 2.39 (s, 3H), 5.05-5.18 (m, 1H), 7.20 (br. s., 1H), 7.43 (d, J=5.39 Hz, 1H), 7.68 (br. s., 1H), 8.17 (br. s., 1H), 8.36 (d, J=6.52 Hz, 1H), 8.49 (d, J=5.37 Hz, 1H), 9.08 (d, J=6.49 Hz, 1H), 9.15 (s, 1H), 9.40 (s, 1H).

### EXAMPLE 101

### N-Benzyl-3-[4-(1-isopropyl-1H-pyrrolo[2,3-c]pyridin-3-yl)-pyrimidin-2-ylamino]-4-methoxy-benzamide [101] (Hereinafter Referred to as “Compound [101]”)

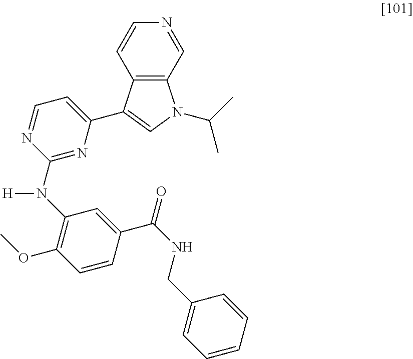

Compound [101] was prepared using a procedure similar to that of Example 44.

Data for Compound [101]: LCMS m/e 493 (M+H); ¹H NMR (400 MHz, Methanol-d₄) δ ppm 1.70 (d, J=6.69 Hz, 6H), 4.00 (s, 3H), 4.54 (s, 2H), 5.00-5.14 (m, 1H), 7.11-7.32 (m, 6H), 7.41 (d, J=5.37 Hz, 1H), 7.64 (dd, J=8.52, 2.17 Hz, 1H), 8.10 (d, J=6.44 Hz, 1H), 8.45 (d, J=5.37 Hz, 1H), 8.83-8.90 (m, 2H), 9.15 (s, 1H), 9.31 (s, 1H).

### EXAMPLE 102

### 4-(2-Dimethylamino-ethoxy)-3-[4-(1-isopropyl-1H-pyrrolo[2,3-c]pyridin-3-yl)-pyrimidin-2-ylamino]-benzonitrile [102] (Hereinafter Referred to as “Compound [102]”)

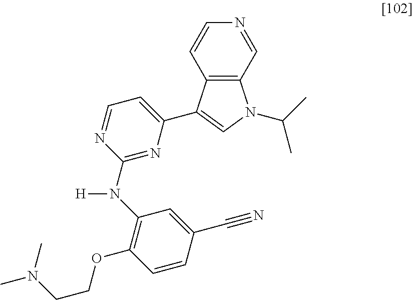

Compound [102] was prepared using a procedure similar to that of Example 44.

Data for Compound [102]: LCMS m/e 442 (M+H); ¹H NMR (400 MHz, Methanol-d₄) δ ppm 1.72 (d, J=6.69 Hz, 6H), 3.03 (s, 6H), 3.74 (m,2H), 4.58 (m,2H), 5.04-5.23 (m, 1H), 7.31 (d, J=8.54 Hz, 1H), 7.53-7.60 (m, 2H), 8.39 (d, J=6.49 Hz, 1H), 8.53 (d, J=5.61 Hz, 1H), 8.72 (d, J=1.81 Hz, 1H), 9.23 (s, 1H), 9.46 (s, 1H).

### EXAMPLE 103

### 4-[4-(1-Isopropyl-1H-pyrrolo[2,3-c]pyridine-3-yl)-pyrimidin-2-ylamino]-N,N-dimethyl-benzamide [103] (Hereinafter Referred to as “Compound [103]”)

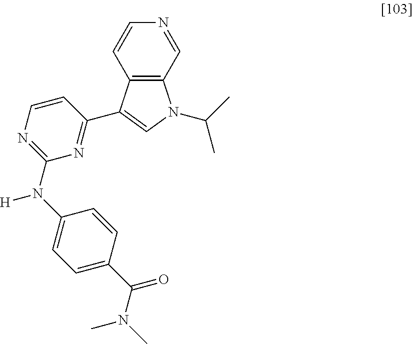

### (1) Trifluoromethanesulfonic acid 4-(1-isopropyl-1H-pyrrolo[2,3-c]pyridine-3-yl)-pyrimidin-2-yl ester (E-7c)

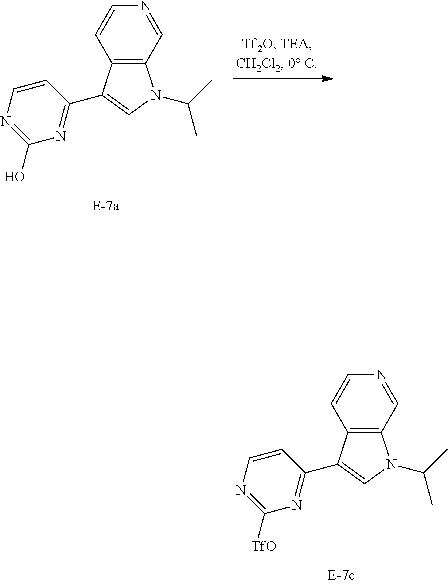

Compound E-7a (0.2600 g, 1.022 mmol, 1.0 eq) was dissolved in CH₂Cl₂ (10 mL) and the solution was cooled to 0° C. in an ice bath. To this solution was first added Et₃N (0.4273 mL, 3.066 mmol, 3.0 eq) followed by the addition of triflic anhydride (0.3439 mL, 2.044 mmol, 2.0 eq). The reaction stirred for 45 minutes and then quenched at 0° C. with water. The product was then extracted with CH₂Cl₂ (4×30 mL). The combined organic layers were washed with brine (2×75 mL) and dried over MgSO₄. The solution was filtered by gravity and then concentrated under reduced pressure. Column chromatography using silica gel (gradient 0% to 10% MeOH in CH₂Cl₂) provided Compound E-7c which was used without further characterization or purification: LCMS m/e 387.

### (2) 4-[4-(1-Isopropyl-1H-pyrrolo[2,3-c]pyridine-3-yl)-pyrimidin-2-ylamino]-N,N-dimethyl-benzamide [103]

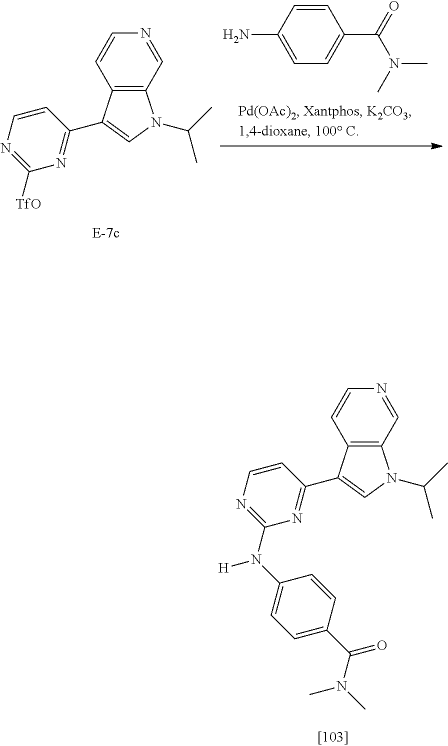

The triflate E-7c (22.1 mg, 0.05720 mmol, 1.0 eq) was combined with 4-(amino)-N,N-dimethyl benzamide (11.27 mg, 0.06864 mmol, 1.2 eq), palladium(II)acetate (1.28 mg, 0.005720 mmol, 0.1 eq), Xantphos (6.62 mg, 0.01144 mmol, 0.2 eq) and potassium carbonate (180.9 mg, 1.1440 mmol, 20 eq). To this was added degassed 1,4-dioxane (3.0 mL). The vessel was then sealed, evacuated, and flushed with N₂. The reaction was heated to 100° C. and stirred for 18 hours. The reaction was then cooled. The solvent was removed under reduced pressure. The residue was taken up in 4 mL of methanol and filtered through a 0.45 micron syringe filter. Purification by reverse phase preparatory HPLC provided Compound [103].

Data for Compound [103]: LCMS m/e 401; ¹H NMR (400 MHz, Methanol-d₄) δ ppm 1.72 (s, 3H), 1.76 (s, 3H), 3.14 (s, 6H), 5.10-5.19 (m, 1H), 7.40-7.46 (m, 1H), 7.46-7.53 (m, 2H), 7.81-7.91 (m, 2H), 8.34-8.44 (m, 1H), 8.47-8.57 (m, 1H), 9.11-9.18 (m, 2H), 9.37-9.45 (m, 1H).

### EXAMPLE 104

### (4-Isopropoxy-phenyl)-[4-(1-isopropyl-1H-pyrrolo[2,3-c]pyridine-3-yl)-pyrimidin-2-yl]-amine [104] (Hereinafter Referred to as “Compound [104]”)

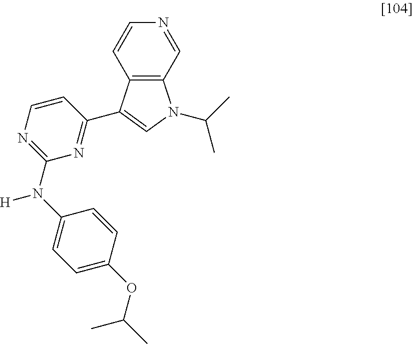

Compound [104] was prepared using a procedure similar to that of Example 103.

Data for Compound [104]: LCMS m/e 388 (M+H); ¹H NMR (400 MHz, Methanol-d₄) δ ppm 1.37 (s, 3H), 1.38 (s, 3H), 1.73 (s, 3H), 1.75 (s, 3H), 4.65 (dt, J=12.1, 6.1 Hz, 1H), 5.14 (dt, J=13.4, 6.7 Hz, 1H), 7.03 (d, J=8.9 Hz, 2H), 7.44 (d, J=5.8 Hz, 1H), 7.50 (d, J=8.9 Hz, 2H), 8.32 (t, J=6.2 Hz, 2H), 8.98 (d, J=6.1 Hz, 0H), 9.24 (s, 1H), 9.43 (s, 1H).

### EXAMPLE 105

### [4-(1-Isopropyl-1H-pyrrolo[2,3-c]pyridine-3-yl)-pyrimidin-2-yl]-(4-pyrrolidin-1-yl-phenyl)-amine [105] (Hereinafter Referred to as “Compound [105]”)

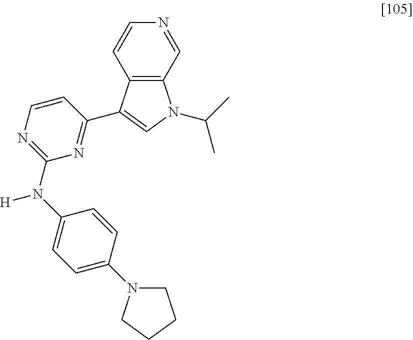

Compound [105] was prepared using a procedure similar to that of Example 103.

Data for Compound [105]: LCMS m/e 399; ¹H NMR (400 MHz, Methanol-d₄) δ ppm 1.73 (s, 3H), 1.75 (s, 3H), 2.17 (br. s., 4H), 3.52 (br. s., 4H), 5.15 (dt, J=13.2, 6.5 Hz, 1H), 7.02 (d, J=7.4 Hz, 2H), 7.55 (d, J=7.7 Hz, 3H), 8.29 (d, J=5.8 Hz, 1H), 8.38 (d, J=6.5 Hz, 1H), 9.06 (d, J=5.6 Hz, 1H), 9.33 (s, 1H), 9.48 (s, 1H).

### EXAMPLE 106

### 3-[4-(1-Isopropyl-1H-pyrrolo[2,3-c]pyridine-3-yl)-pyrimidin-2-ylamino]-benzoic acid methyl ester [106] (Hereinafter Referred to as “Compound [106]”)

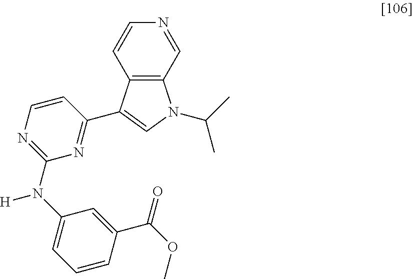

Compound [106] was prepared using a procedure similar to that of Example 103.

Data for Compound [106]: LCMS m/e 388 (M+H); ¹H NMR (400 MHz, Methanol-d₄) δ ppm 1.74 (s, 3H), 1.76 (s, 3H), 3.93 (s, 3H), 5.15 (dt, J=13.4, 6.8 Hz, 1H), 7.45 (d, J=5.4 Hz, 1H), 7.49 (t, J=7.9 Hz, 1H), 7.75 (d, J=7.8 Hz, 1H), 7.84 (d, J=8.3 Hz, 1H), 8.32 (d, J=6.5 Hz, 1H), 8.51 (d, J=5.3 Hz, 1H), 8.62 (s, 0H), 9.11 (d, J=6.5 Hz, 1H), 9.19 (s, 1H), 9.42 (s, 1H).

### EXAMPLE 107

### 4-[4-(1-Isopropyl-1H-pyrrolo[2,3-c]pyridine-3-yl)-pyrimidin-2-yl-amino]-N-methyl-benzamide [107] (Hereinafter Referred to as “Compound [107]”)

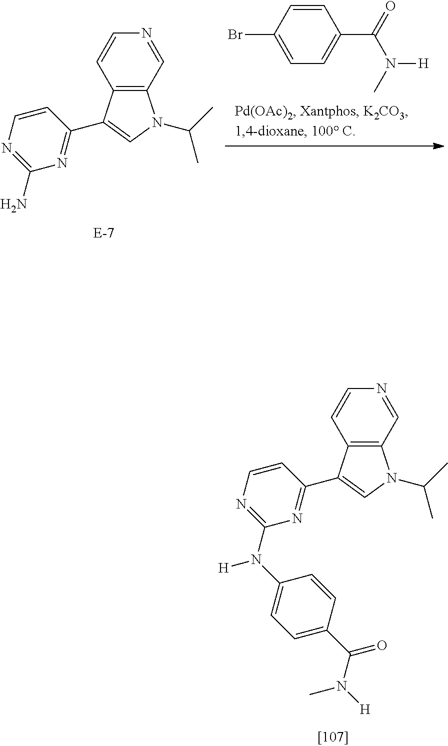

The aminopyrimidine derivative E-7 (81.6 mg, 0.3221 mmol, 1.2 eq) was combined with 4-bromo N-methyl-benzamide (57.5 mg, 0.2684 mmol, 1.0 eq), palladium(II) acetate (6.0 mg, 0.02684 mmol, 0.1 eq), Xantphos (31.1 mg, 0.5368 mmol, 2.0 eq) and potassium carbonate (371 mg, 2.684 mmol, 20.0 eq). To this was added previously degassed 1,4-dioxane (2.0 mL) in a sealed vessel. The vial was flushed with N₂ and sealed. The reaction was heated to 100° C. and allowed to stir for 18 hours. The reaction was then cooled, diluted with methanol (2.0 mL), and filtered through a 0.2 micron syringe filter. Purification by reverse phase preparatory HPLC afforded Compound [107].

Data for Compound [107]: LCMS m/e 387 (M+H); ¹H NMR (400 MHz, Methanol-d₄) δ ppm 1.70 (s, 3H), 1.71 (s, 3H), 2.93 (s, 3H), 5.10 (dt, J=13.3, 6.6 Hz, 1H), 7.44 (d, J=5.5 Hz, 1H), 7.75-7.85 (m, 4H), 8.35 (d, J=6.5 Hz, 1H), 8.44 (d, J=5.5 Hz, 1H), 9.05 (d, J=6.4 Hz, 1H), 9.15 (s, 1H), 9.38 (s, 1H).

### EXAMPLE 108

### 4-[4-(1-Isopropyl-1H-pyrrolo[2,3-c]pyridine-3-yl)-pyrimidin-2-ylamino]-benzoic acid methyl ester [108] (Hereinafter Referred to as “Compound [108]”)

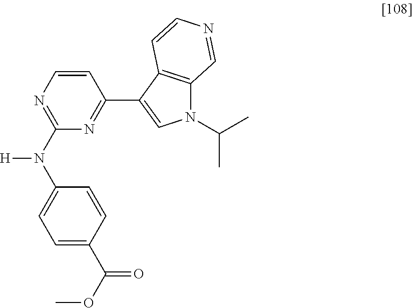

Compound [108] was prepared using a procedure similar to that of Example 107.

Data for Compound [108]: LCMS m/e 388 (M+H); ¹H NMR (400 MHz, Methanol-d₄) δ ppm 1.73 (s, 3H), 1.74 (s, 3H), 3.91 (s, 3H), 5.13 (spt, J=6.6 Hz, 1H), 7.46 (d, J=5.4 Hz, 1H), 7.87 (d, J=8.8 Hz, 2H), 7.99 (d, J=8.7 Hz, 2H), 8.37 (d, J=6.5 Hz, 1H), 8.52 (d, J=5.4 Hz, 1H), 9.13-9.20 (m, 2H), 9.41 (s, 1H).

### EXAMPLE 109

### 3-[4-(1-Isopropyl-1H-pyrrolo[2,3-c]pyridine-3-yl)-pyrimidin-2-ylamino]-benzoic acid tert-butyl ester [109] (Hereinafter Referred to as “Compound [109]”)

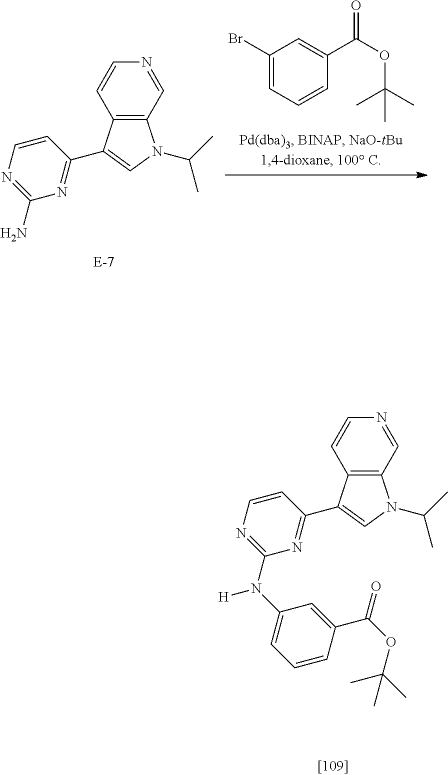

The aminopyrimidine derivative E-7 (58.8 mg, 0.2321 mmol, 1.0 eq) was combined with 3-bromo-benzoic acid t-butyl ester (71.6 mg, 0.2785 mmol, 1.2 eq), tris(dibenzylideneacetone)dipalladium (10.6 mg, 0.01160 mmol, 0.05 eq), BINAP (14.4 mg, 0.02321 mmol, 0.10 eq), and sodium t-butoxide (66.9 mg, 0.6963 mmol, 3.0 eq) in a sealed tube. To this was added previously degassed 1,4-dioxane (2.5 mL). The vial was flushed with N₂ and heated to 100° C. The reaction was stirred for 18 hours. The reaction vessel was cooled and diluted with methanol (2.5 mL). The solution was passed through a 0.2 micron syringe filter. Purification by reverse phase preparatory HPLC provided compound Compound [109].

Data for Compound [109]: LCMS m/e 430 (M+H); ¹H NMR (400 MHz, Methanol-d₄) δ ppm 1.58 (s, 9H), 1.71 (s, 3H), 1.73 (s, 3H), 5.03-5.19 (m, 1H), 7.46 (t, J=7.9 Hz, 2H), 7.71 (d, J=7.8 Hz, 1H), 7.76 (dd, J=8.0, 1.0 Hz, 1H), 8.28 (d, J=6.5 Hz, 1H), 8.44 (d, J=5.5 Hz, 1H), 8.47 (s, 1H), 8.96 (d, J=6.4 Hz, 1H), 9.21 (s, 1H), 9.40 (s, 1H).

### EXAMPLE 110

### 4-Chloro-3-[4-(1-isopropyl-1H-pyrrolo[2,3-c]pyridine-3-yl)-pyrimidin-2-ylamino]-benzonitrile [110] (Hereinafter Referred to as “Compound [110]”)

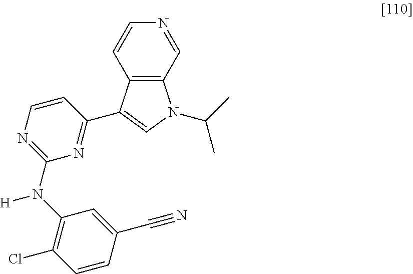

Compound [110] was prepared using a procedure similar to that of Example 109.

Data for Compound [110]: LCMS m/e 389 (M+H); ¹H NMR (400 MHz, Methanol-d₄) δ ppm 1.74 (s, 3H), 1.75 (s, 3H), 5.15 (dt, J=13.3, 6.7 Hz, 1H), 7.50 (d, J=9.7 Hz, 1H), 7.56 (d, J=5.3 Hz, 1H), 7.73 (d, J=8.2 Hz, 1H), 8.39 (d, J=6.5 Hz, 1H), 8.57 (d, J=5.3 Hz, 1H), 8.80 (s, 1H), 8.95 (d, J=6.5 Hz, 1H), 9.20 (s, 1H), 9.44 (s, 1H).

### EXAMPLE 111

### 3-[4-(1-Isopropyl-1H-pyrrolo[2,3-c]pyridin-3-yl)-pyrimidin-2-ylamino]-N-methyl-benzamide [111] (Hereinafter Referred to as “Compound [111]”)

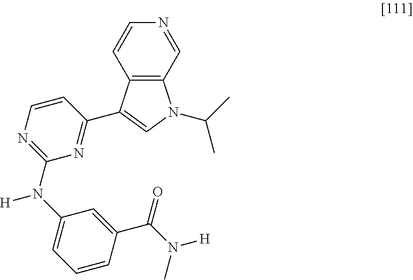

Compound [111] was prepared using a procedure similar to that of Example 109.

Data for Compound [111]: LCMS m/e 387 (M+H); ¹H NMR (400 MHz, Methanol-d₄) δ ppm 1.71 (s, 3H), 1.73 (s, 3H), 2.92 (d, J=4.2 Hz, 3H), 5.12 (dt, J=13.3, 6.6 Hz, 1H), 7.41-7.54 (m, 4H), 7.57 (d, J=7.7 Hz, 1H), 7.71 (d, J=7.3 Hz, 1H), 7.80 (d, J=7.4 Hz, 1H), 8.29 (d, J=4.1 Hz, 2H), 8.44 (d, J=5.6 Hz, 1H), 8.99 (d, J=6.3 Hz, 1H), 9.22 (s, 1H), 9.41 (s, 1H).

### EXAMPLE 112

### 3-[4-(1-Isopropyl-1H-pyrrolo[2,3-c]pyridine-3-yl)-pyrimidin-2-ylamino]-N,N-dimethyl-benzamide [112] (Hereinafter Referred to as “Compound [112]”)

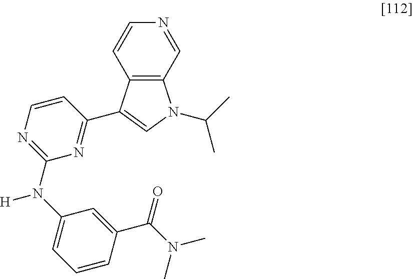

Compound [112] was prepared using a procedure similar to that of Example 109.

Data for Compound [112]: LCMS m/e 401 (M+H); ¹H NMR (400 MHz, Methanol-d₄) δ ppm 1.66 (s, 3H), 1.67 (s, 3H), 2.98 (s, 3H), 3.05 (s, 3H), 5.00-5.14 (m, 1H), 7.19 (d, J=7.5 Hz, 1H), 7.40-7.52 (m, 2H), 7.58 (d, J=9.0 Hz, 1H), 7.84 (s, 1H), 8.31 (d, J=6.5 Hz, 1H), 8.35 (d, J=5.9 Hz, 1H), 8.86 (d, J=6.3 Hz, 1H), 9.19 (s, 1H), 9.36 (s, 1H).

### EXAMPLE 113

### 3-Bromo-5-[4-(1-isopropyl-1H-pyrrolo[2,3-c]pyridin-3-yl)-pyrimidin-2-ylamino]-N-methyl-benzamide [113] (Hereinafter Referred to as “Compound [113]”)

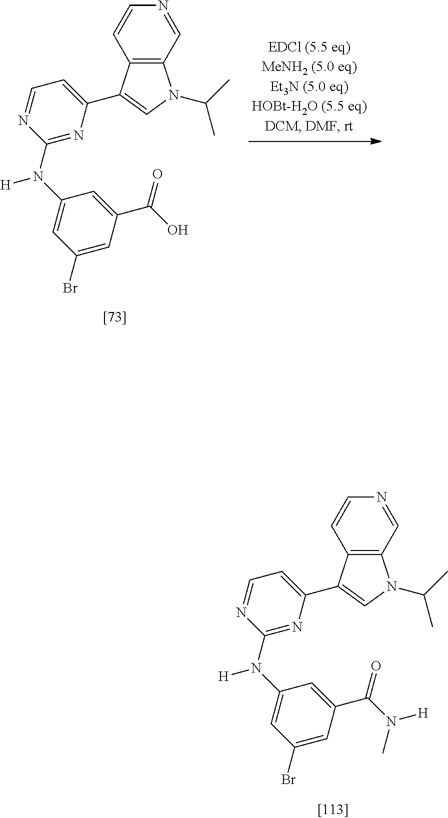

Compound [73] (40 mg, 0.088 mmol) was dissolved in DCM (1 mL), CH₃CN (1 mL), and DMF (2 mL). To this solution was added Et₃N (44 mg, 0.44 mmol, 5.0 eq), EDCI (18.6 mg, 0.097 mmol, 1.1 eq), HOBt-H₂O (14 mg, 0.097 mmol, 1.1 eq), and MeNH₂ (0.044 mL, 0.088 mmol, 1.0 eq). After 2 hours, EDCI (75 mg, 0.39 mmol, 4.4 eq), HOBt-H₂O (64 mg, 00.40 mmol, 4.4 eq), and MeNH₂ (0.18 mL, 0.34 mmol, 4.0 eq) were added. After 22 hours, the reaction was deemed complete. The reaction was concentrated. The residue was dissolved in 80% MeOH in water (4 mL). The solution was purified by preparative reverse-phase preparative HPLC to provide Compound [113] (2.2 mg, 5.5%). Data for Compound [113]: LCMS m/e 466 (M+H); ¹H NMR (400 MHz, Methanol-d₄) δ ppm 1.71 (s, 3H), 1.73 (s, 3H), 2.90 (s, 3H), 5.06-5.16 (m, 1H), 7.44 (d, J=5.30 Hz, 1H), 7.59 (t, J=1.56 Hz, 1H), 8.15 (t, J=1.70 Hz, 1H), 8.21 (t, J=1.84 Hz, 1H), 8.32 (d, J=6.52 Hz, 1H), 8.51 (d, J=5.30 Hz, 1H), 9.08 (d, J=6.44 Hz, 1H), 9.14 (s, 1H), 9.38 (s, 1H).

### EXAMPLE 114

### 3-[4-(1-Isopropyl-1H-pyrrolo[2,3-c]pyridin-3-yl)-pyrimidin-2-ylamino]-5-methoxy-benzamide [114] (Hereinafter Referred to as “Compound [114]”)

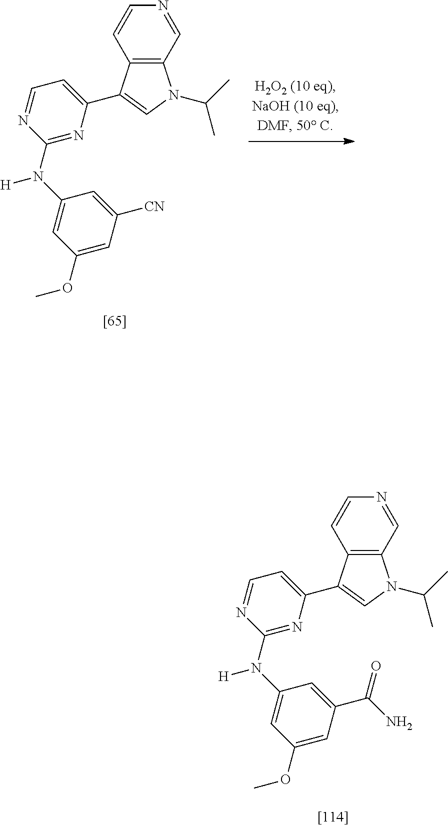

Compound [65] (38 mg, 0.10 mmol) was dissolved in DMF (1 mL). To this solution was added 5 M aqueous NaOH (0.1 mL, 0.5 mmol, 5 eq) and 30% aqueous H₂O₂ (0.10 mL, 1.0 mmol, 10 eq). The mixture was heated at 50° C. for 3 hours and the reaction was deemed complete. The reaction was then cooled to room temperature and treated with saturated Na₂SO₃ (5 mL). The mixture was extracted with EtOAc (three times 10 mL) and dried over anhydrous MgSO₄. After filtration, the mixture was concentrated. Purification by reverse-phase preparatory HPLC provided Compound [114] (10.7 mg) as a yellowish solid.

Data for Compound [114]: LCMS m/e 403 (M+H); ¹H NMR (400 MHz, Methanol-d₄) δ ppm 1.71 (s, 3H), 1.73 (s, 3H), 3.88 (s, 3H), 5.07-5.16 (m, 1H), 7.16-7.18 (m, 1H), 7.41-7.47 (m, 2H), 7.88 (t, J=1.54 Hz, 1H), 8.29 (d, J=6.49 Hz, 1H), 8.46 (d, J=5.47 Hz, 1H), 9.08 (d, J=6.49 Hz, 1H), 9.17 (s, 1H), 9.39 (s, 1H).

### EXAMPLE 115

### 3-[4-(1-Isopropyl-1H-pyrrolo[2,3-c]pyridin-3-yl)-pyrimidin-2-ylamino]-5-trifluoromethoxy-benzamide [115] (Hereinafter Referred to as “Compound [115]”)

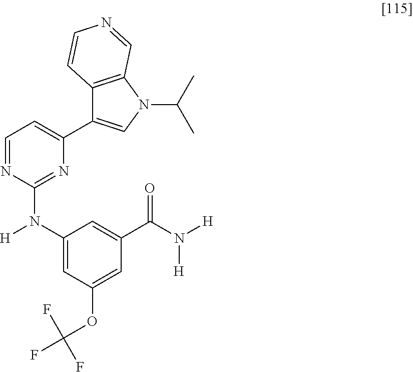

Compound [115] was prepared using a procedure similar to that of Example 114.

Data for Compound [115]: LCMS m/e 457 (M+H); ¹H NMR (400 MHz, Methanol-d₄) δppm 1.70 (s, 3H), 1.72 (s, 3H), 5.05-5.17 (m, 1H), 7.40 (br. s., 1H), 7.45 (d, J=5.39 Hz, 1H), 7.95 (br. s., 1H), 8.27 (br. s., 1H), 8.31 (d, J=6.52 Hz, 1H), 8.50 (d, J=5.37 Hz, 1H), 9.08 (d, J=6.49 Hz, 1H), 9.14 (s, 1H), 9.39 (s, 1H).

### EXAMPLE 116

### 3-[4-(1-Isopropyl-1H-pyrrolo[2,3-c]pyridin-3-yl)-pyrimidin-2-ylamino]-5-methyl-benzamide [116] (Hereinafter Referred to as “Compound [116]”)

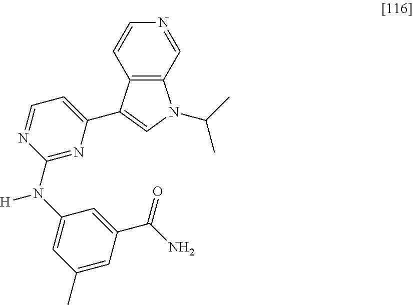

Compound [116] was prepared using a procedure similar to that of Example 114.

Data for Compound [116]: LCMS m/e 387 (M+H); ¹H NMR (400 MHz, Methanol-d₄) δppm 1.70 (s, 3H), 1.72 (s, 3H), 2.43 (s, 3H), 5.04-5.16 (m, 1H), 7.42-7.46 (m, 2H), 7.58 (br. s., 1H), 8.12 (br. s., 1H), 8.28 (d, J=6.52 Hz, 1H), 8.43 (d, J=5.54 Hz, 1H), 9.02 (d, J=6.44 Hz, 1H), 9.18 (s, 1H), 9.38 (s, 1H).

### EXAMPLE 117

### 3-[4-(1-Isopropyl-1H-pyrrolo[2,3-c]pyridin-3-yl)-pyrimidin-2-ylamino]-4-methoxy-N-(2-methoxy-ethyl)-benzamide [117] (Hereinafter Referred to as “Compound [117]”)

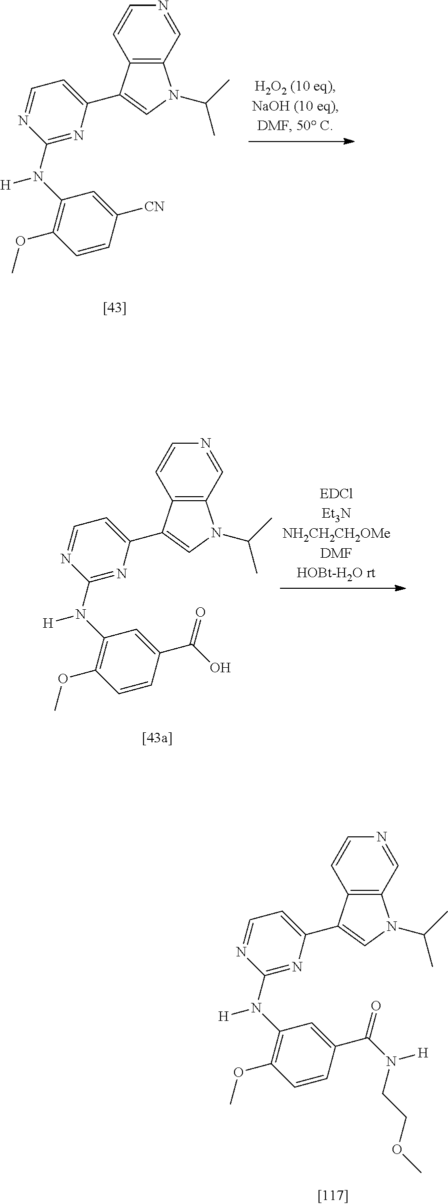

Compound [43] (0.24 g, 0.62 mmol) was dissolved in EtOH (6 mL) and 6 N KOH (6 mL, 36 mmol, 58 eq). The mixture was heated at 80° C. for overnight and additional KOH (2.0 g, 35.7 mmol, 58 eq) was added. The mixture was heated to reflux for 16 hours and the reaction was deemed complete. The reaction was then cooled to rt and treated with saturated water (10 mL), extracted with EtOAc (three times 15 mL), and the organic phases dried over anhydrous MgSO₄. After filtration, the mixture was concentrated. Purification through silica gel chromatography provided Compound [43a] (0.25 g) as a yellowish solid.

Using Compound [43a] as the starting material, Compound [117] was prepared using an amidation procedure similar to that for Compound [113].

Data for Compound [117]: LCMS m/e 461 (M+H); ¹H NMR (400 MHz, Methanol-d₄) δ ppm 1.70 (s, 3H), 1.72 (s, 3H), 3.34 (s, 3H), 3.52-3.57 (m, 4H), 3.96 (s, 3H), 5.06-5.15 (m, 1H), 7.20 (d, J=8.64 Hz, 1H), 7.55 (d, J=6.10 Hz, 1H), 7.77 (dd, J=8.59, 2.22 Hz, 1H), 8.29 (d, J=6.49 Hz, 1H), 8.38 (d, J=6.05 Hz, 1H), 8.48 (d, J=2.15 Hz,1H), 8.79 (d, J=6.44 Hz, 1H), 9.28 (s, 1H), 9.42 (s, 1H).

### EXAMPLE 118

### 3-[4-(1-Isopropyl-1H-pyrrolo[2,3-c]pyridin-3-yl)-pyrimidin-2-ylamino]-4-methoxy-N-piperidin-4-yl-benzamide [118] (Hereinafter Referred to as “Compound [118]”)

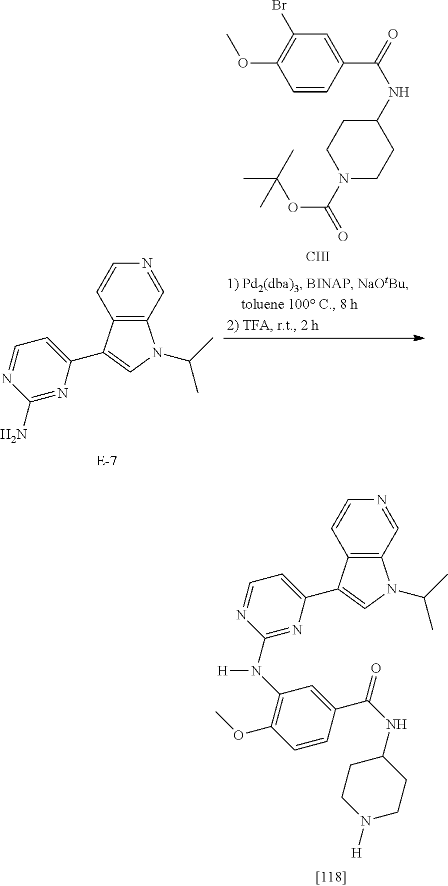

To a 40 mL scintillation vial fitted with a magnetic stir bar was added the starting material E-7 (0.050 g, 0.198 mmol, 1 eq.) and N₂ was bubbled through the vessel for 10 minutes. To the vessel was added toluene (5 mL) followed by the addition of the bromide CIII (0.097 g, 0.237 mmol, 1.2 eq.) and N₂ bubbled through the solution for an additional 5 minutes. BINAP (0.012 g, 0.0198 mmol, 0.10 eq.), sodium tert-butoxide (0.057 g, 0.594 mmol, 3 eq.), and Pd₂(dba)₃ (0.009 g, 0.0099 mmol, 0.05 eq.) were then added sequentially. The reaction was stirred 16 hours under N₂ at 100° C. The reaction mixture was then cooled, concentrated to dryness, and the residue was stirred with TFA (2 mL) at room temperature for 2 hours. The mixture was concentrated and re-dissolved in methanol (5 mL) and purified by reverse-phase preparatory HPLC using acetonitrile and water with 0.05% TFA as the eluent to provide Compound [118].

Data for Compound [118]: LCMS m/e 486 (M+H); ¹H NMR (400 MHz, Methanol-d₄) δ ppm 1.71 (s, 3H), 1.73 (s, 3H), 1.78-1.93 (m, 2H), 2.18 (d, J=11.47 Hz, 2H), 3.05-3.23 (m, 2H), 3.42-3.51 (m, 2H), 3.99 (s, 3H), 4.09-4.20 (m, 1H), 5.05-5.17 (m, 1H), 7.18 (d, J=8.64 Hz, 1H), 7.48 (d, J=5.66 Hz, 1H), 7.70 (dd, J=8.54, 2.10 Hz, 1H), 8.30 (d, J=6.44 Hz, 1H), 8.43 (d, J=5.61 Hz, 1H), 8.64 (d, J=1.95 Hz, 1H), 8.92 (d, J=6.49 Hz, 1H), 9.23 (s, 1H), 9.41 (s, 1H).

### EXAMPLE 119

### 5-[4-(1-Isopropyl-1H-pyrrolo[2,3-c]pyridin-3-yl)-pyrimidin-2-ylamino]-isophthalonitrile [119] (Hereinafter Referred to as “Compound [119]”)

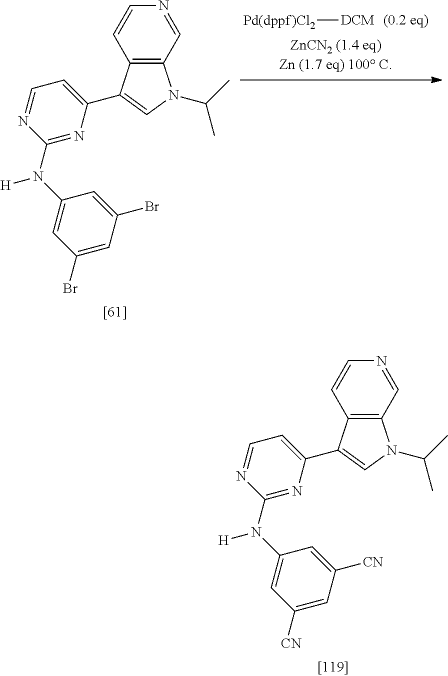

To a solution of Compound [61] (36 mg, 0.074 mmol) in DMF (2 mL) was added ZnCN₂ (12 mg, 0.10 mmol, 1.4 eq) and Zn (8 mg, 0.12 mmol, 1.7 eq). The reaction vessel was evacuated and refilled with nitrogen. This process was carried out three times, then Pd(dppf)Cl₂-DCM (12 mg, 0.015 mmol, 0.2 eq) was added. The solution was evacuated and filled with nitrogen again, and the vial sealed and heated at 100° C. for 2 hours. The reaction was then cooled to room temperature and dissolved in MeOH (4 mL). The solution was filtered through a 0.45 micron syringe filter. Purification by reverse-phase preparative HPLC provided Compound [119] (0.6 mg) as a white solid.

Data for Compound [119]: LCMS m/e 380 (M+H); ¹H NMR (400 MHz, Methanol-d₄) δ ppm 1.71 (s, 3H) 1.73 (s, 3H) 5.01-5.21 (m, 1H) 7.52 (d, J=5.30 Hz, 1H) 7.73 (t, J=1.35 Hz, 1H) 8.40 (d, J=6.49 Hz, 1H) 8.50 (s, 1H) 8.51 (s, 1H) 8.59 (d, J=5.32 Hz, 1H) 9.11 (d, J=6.35 Hz, 1H) 9.17 (s, 1H) 9.41 (s, 1H).

### EXAMPLE 120

### 3-[4-(1-Isopropyl-1H-pyrrolo[2,3-c]pyridin-3-yl)-pyrimidin-2-ylamino]-5-trifluoromethoxy-benzonitrile [120] (Hereinafter Referred to as “Compound [120]”)

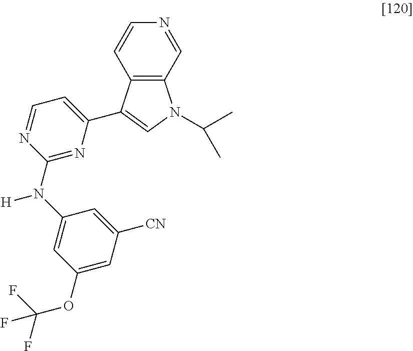

Compound [120] was prepared using a procedure similar to that of Example 119.

Data for Compound [120]: LCMS m/e 439 (M+H); ¹H NMR (400 MHz, Methanol-d₄) δppm 1.71 (s, 3H), 1.73 (s, 3H), 5.07-5.17 (m, 1H), 7.26 (br. s., 1H), 7.49 (d, J=5.32 Hz, 1H), 8.08 (br. s., 1H), 8.31 (t, J=1.51 Hz, 1H), 8.38 (d, J=6.52 Hz, 1H), 8.57 (d, J=5.32 Hz, 1H), 9.14 (d, J=6.61 Hz, 1H), 9.15 (s, 1H), 9.41 (s, 1H).

### EXAMPLE 121

### 4-Hydroxy-3-[4-(1-isopropyl-1H-pyrrolo[2,3-c]pyridin-3-yl)-pyrimidin-2-ylamino]-benzamide [121] (Hereinafter Referred to as “Compound [121]”)

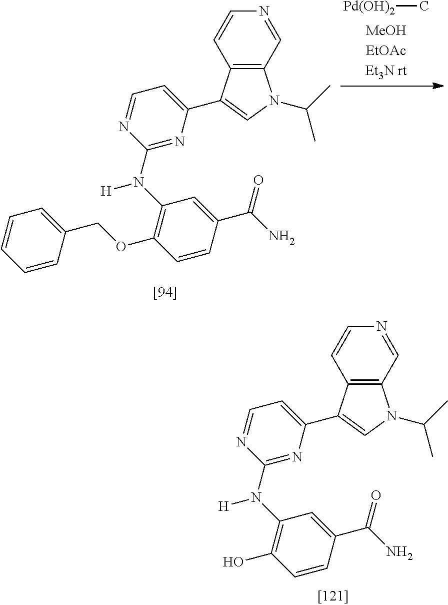

Compound [94] (10 mg, 0.021 mmol) was dissolved in MeOH (1 mL) and EtOAc (1 mL), Et₃N (1 drop), and Pd(OH)₂-C (10 mg). The reaction vessel was evacuated and refilled with hydrogen. This process was carried out three times. The hydrogenation was carried out via balloon hydrogenation. After 0.5 hour, the reaction was deemed complete. The reaction mixture was filtered through a thin layer of Celite that was washed with wet MeOH. The mixture was concentrated. Purification through a preparative C₁₈ HPLC column provided Compound [121] (1.8 mg) as a white solid.

Data for Compound [121]: LCMS m/e 389 (M+H); ¹H NMR (400 MHz, Methanol-d₄) δppm 1.70 (s, 3H), 1.72 (s, 3H), 5.06-5.15 (m, 1H), 7.00 (d, J=8.44 Hz, 1H), 7.47 (d, J=5.69 Hz, 1H), 7.62 (dd, J=8.41, 2.23 Hz, 1H), 8.27 (d, J=6.44 Hz, 1H), 8.42 (d, J=5.64 Hz, 1H), 8.55 (d, J=2.07 Hz, 1H), 8.94 (d, J=6.42 Hz, 1H), 9.21 (s, 1H), 9.39 (s, 1H).

### EXAMPLE 122

### 3-[4-(1-Isopropyl-1H-pyrrolo[2,3-c]pyridin-3-yl)-pyrimidin-2-ylamino]-5-trifluoromethyl-benzamide [122] (Hereinafter Referred to as “Compound [122]”)

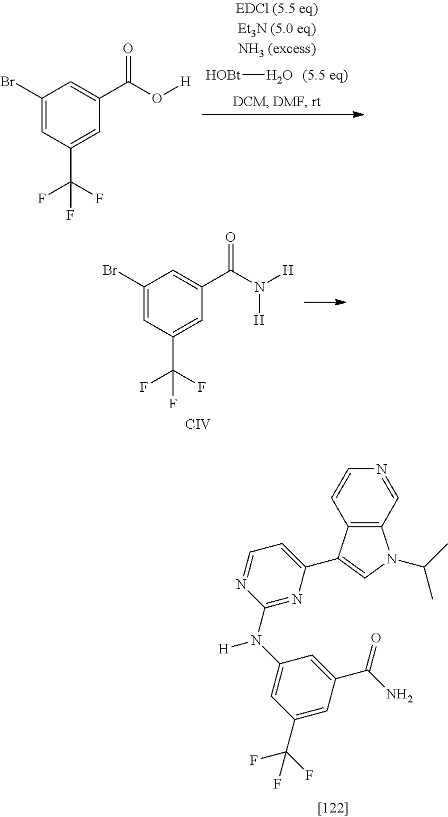

3-Bromo-5-trifluoromethyl benzoic acid (269 mg, 1 mmol) was dissolved in DCM (5 mL). To this solution was added Et₃N (0.51 g, 5.0 mmol, 5.0 eq), EDCI (0.21 g, 1.1 mmol, 1.1 eq), HOBt-H₂O (0.15 g, 1.1 mmol, 1.1 eq), and NH₃ (excess). After 2 hours, the reaction was deemed complete. The reaction mixture was treated water (10 mL), extracted with EtOAc (2×15 mL), and dried over anhydrous MgSO₄. After filtration, the mixture was concentrated onto Celite. Column chromatography using silica gel (gradient 0% to 50% MeOH in DCM) provided Compound CIV (0.20 g) which was used without further characterization.

Using Compound CIV as the starting material, Compound [122] was prepared using a procedure similar to that for the formation of Example 44.

LCMS m/e 441 (M+H); ¹H NMR (400 MHz, Methanol-d₄) δppm 1.71 (s, 3H), 1.72 (s, 3H), 5.07-5.17 (m, 1H), 7.47 (d, J=5.32 Hz, 1H), 7.81 (s, 1H), 8.28-8.33 (m,2H), 8.51-8.56 (m, 2H), 9.06 (d, J=6.39 Hz, 1H), 9.16 (s, 1H), 9.39 (s, 1H).

### EXAMPLE 123

### 3-[4-(1-Isopropyl-1H-pyrrolo[2,3-c]pyridin-3-yl)-pyrimidin-2-ylamino]-N-methyl-5-trifluoromethyl-benzamide [123] (Hereinafter Referred to as “Compound [123]”)

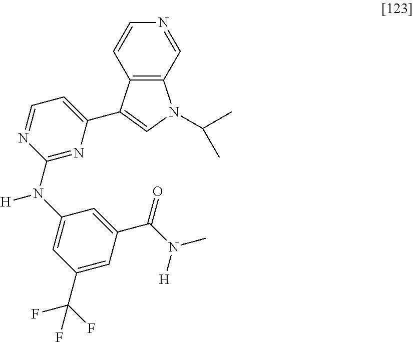

Compound [123] was prepared using a procedure similar to those of Examples 44 and 122.

Data for Compound [123]: LCMS m/e 455 (M+H); ¹H NMR (400 MHz, Methanol-d₄) δppm 1.70 (s, 3H), 1.72 (s, 3H), 2.93 (s, 3H), 5.04-5.15 (m, 1H), 7.44 (d, J=5.47 Hz, 1H), 7.71 (s, 1H),8.22 (s, 1H), 8.26 (d, J=6.47 Hz, 1H), 8.43 (s, 1H), 8.47 (d, J=5.39 Hz, 1H), 8.97 (d, J=6.22 Hz, 1H), 9.13 (s, 1H), 9.38 (s, 1H).

### EXAMPLE 124

### 5-[4-(1-Isopropyl-1H-pyrrolo[2,3-c]pyridin-3-yl)-pyrimidin-2-ylamino]-N,N′-dimethyl-isophthalamide [124] (Hereinafter Referred to as “Compound [124]”)

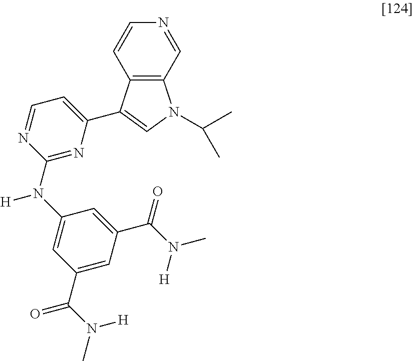

Compound [124] was prepared using a procedure similar to those of Examples 44 and 122.

Data for Compound [124]: LCMS m/e 444 (M+H); ¹H NMR (400 MHz, Methanol-d₄) δppm 1.70 (s, 3H), 1.72 (s, 3H), 2.93 (s, 6H), 5.06-5.16 (m, 1H), 7.46 (d, J=5.54 Hz, 1H), 7.89 (t, J=1.46 Hz, 1H), 8.25 (d, J=6.52 Hz, 1H), 8.30 (s, 1H), 8.30 (s, 1H), 8.47 (d, J=5.49 Hz, 1H), 8.98 (d, J=6.42 Hz, 1H), 9.18 (s, 1H), 9.38 (s, 1H).

### EXAMPLE 125

### 3-Chloro-5-[4-(1-isopropyl-1H-pyrrolo[2,3-c]pyridin-3-yl)-pyrimidin-2-ylamino]-N-methyl-benzamide [125] (Hereinafter Referred to as “Compound [125]”)

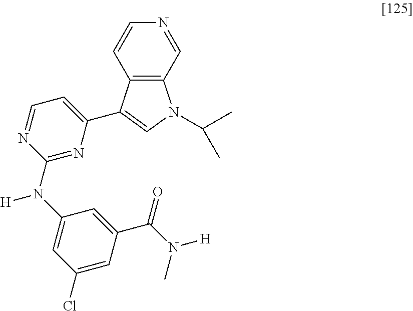

Compound [125] was prepared using a procedure similar to those of Examples 44 and 122.

Data for Compound [125]: LCMS m/e 421 (M+H); ¹H NMR (400 MHz, Methanol-d₄) δ ppm 1.71 (s, 3H), 1.72 (s, 3H), 2.91 (s, 3H), 5.05-5.15 (m, 1H), 7.42-7.46 (m, 2H), 7.99 (br. s., 1H), 8.08 (m, 1H), 8.30 (d, J=6.52 Hz, 1H), 8.47 (d, J=5.42 Hz, 1H), 9.04 (d, J=6.44 Hz, 1H), 9.15 (s, 1H), 9.38 (s, 1H).

### EXAMPLE 126

### N-Cyclohexyl-3-[4-(1-isopropyl-1H-pyrrolo[2,3-c]pyridin-3-yl)-pyrimidin-2-ylamino]-4-methoxy-benzamide [126] (Hereinafter Referred to as “Compound [126]”)

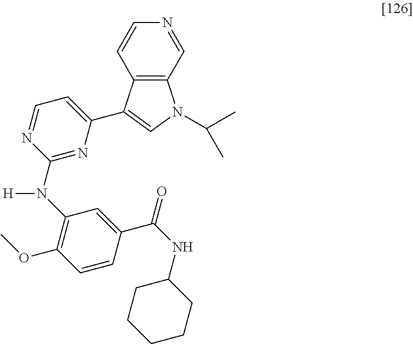

### (1) 3-Bromo-N-cyclohexyl-4-methoxy-benzamide (CV)

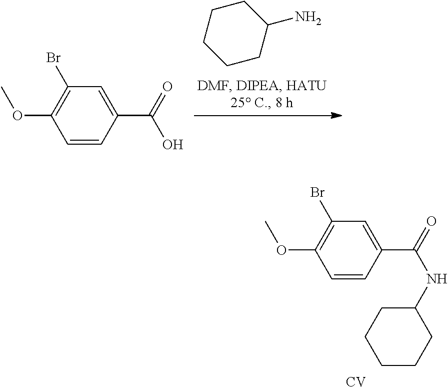

To a solution of 3-bromo-4-methoxy-benzoic acid (0.23 g, 1 mmol, 1.0 eq.) in anhydrous DMF (5 mL) and DIPEA (1.29 g, 10.0 mmol, 10.0 eq.) was added cyclohexylamine (0.198 g, 2.0 mmol, 2.0 eq.) followed by HATU (0.76 g, 2 mmol, 2.0 eq.). The mixture was stirred at 25° C. for 8 hours. The reaction mixture was concentrated and the residue was purified on a silica gel chromatography using CHCl₃/MeOH (10%) as eluent to provide Compound CV: LCMS m/e 312 (M+H); 1H NMR (400 MHz, Chloroform-d) δ ppm 1.13-1.30 (m, 4H), 1.63-1.71 (m, 1H), 1.70-1.82 (m, 2H), 1.95-2.11 (m, 2H), 3.94 (s, 3H), 5.83 (br. s., 1H), 6.92 (d, J=8.54 Hz, 1H), 7.73 (dd, J=8.59, 2.15 Hz, 1H), 7.93 (d, J=2.15 Hz, 1H).

### (2) N-Cyclohexyl-3-[4-(1-isopropyl-1H-pyrrolo[2,3-c]pyridin-3-yl)-pyrimidin-2-ylamino]-4-methoxy-benzamide [126]

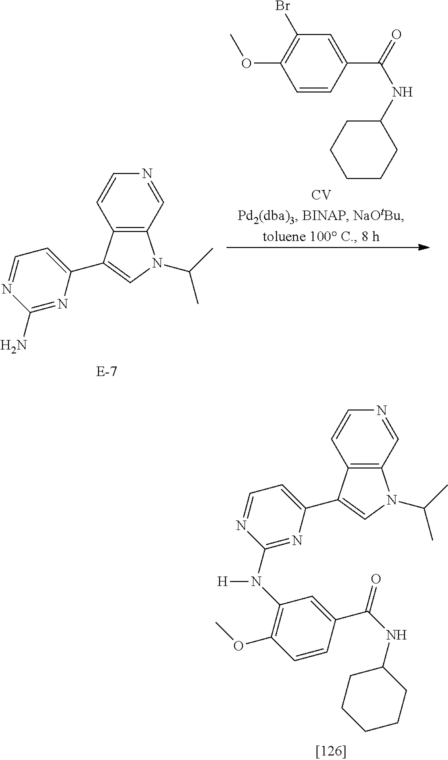

Procedure similar to that for Example 44: To a 40 mL scintillation vial fitted with a magnetic stir bar was added the starting material E-7 (0.050 g, 0.198 mmol, 1 eq.) and N₂ was bubbled through the vessel for 10 minutes. To the vessel was added toluene (5 mL) followed by the addition of the bromide CV (0.074 g, 0.237 mmol, 1.2 eq.), and N₂ was bubbled through the solution for an additional 5 minutes. BINAP (0.012 g, 0.0198 mmol, 0.10 eq.), sodium tert-butoxide (0.057 g, 0.594 mmol, 3 eq.), and Pd₂(dba)₃ (0.009 g, 0.0099 mmol, 0.05 eq.) were then added sequentially. The reaction was stirred 16 hours under N₂ at 100° C. The reaction mixture was then cooled, concentrated to dryness, and the residue re-dissolved in methanol (5 mL), filtered through a 0.2 micron syringe filter, and purified by reverse-phase preparatory HPLC using acetonitrile and water with 0.05% TFA as the eluent to provide Compound [126]. Data for Compound [126]: LCMS m/e 485 (M+H); ¹H NMR (400 MHz, Methanol-d₄) δ ppm 1.14-1.44 (m, 6H), 1.78 (d, J=12.79 Hz, 2H), 1.88 (d, J=10.84 Hz, 2H), 3.77-3.88 (m, 1H), 5.05-5.17 (m, 1H), 7.17 (d, J=8.64 Hz, 1H), 7.49 (d, J=5.76 Hz, 1H), 7.69 (dd, J=8.54, 2.15 Hz, 1H), 8.25 (d, J=6.49 Hz, 1H), 8.41 (d, J=5.76 Hz, 1H), 8.58 (d, J=2.10 Hz, 1H), 8.86 (d, J=6.44 Hz, 1H), 9.24 (s, 1H), 9.41 (s, 1H).

### EXAMPLE 127

### 3-[4-(1-Isopropyl-1H-pyrrolo[2,3-c]pyridin-3-yl)-pyrimidin-2-ylamino]-4-methoxy-N-(4-methyl-cyclohexyl)-benzamide [127] (Hereinafter Referred to as “Compound [127]”)

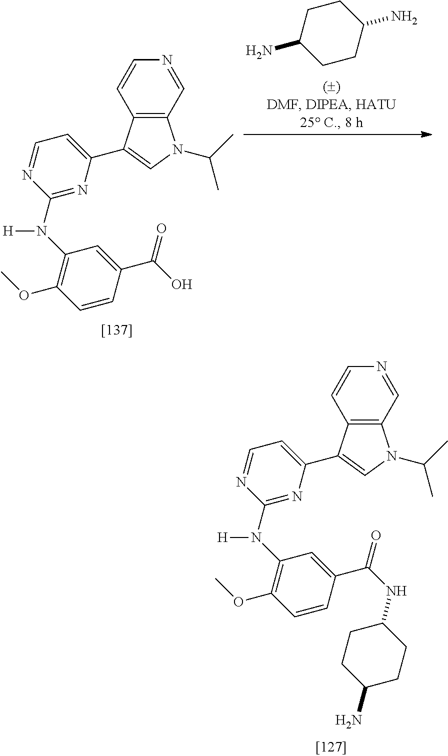

To a solution of Compound [137] (0.025 g, 0.062 mmol, 1.0 eq.) in anhydrous DMF (2 mL) and DIPEA (0.08 g, 0.62 mmol, 10.0 eq.) was added (±)-trans-1,4-diaminocyclohexane (0.014 g, 0.124 mmol, 2.0 eq.) followed by HATU (0.0706 g, 0.186 mmol, 3.0 eq.), and the mixture was stirred at 25° C. for 8 hours. The reaction mixture was concentrated, and the residue was dissolved in methanol and purified on a reverse phase preparatory PLC using acetonitrile and water with 0.05% TFA as the eluent to provide Compound [127].

Data for Compound [127]: LCMS m/e 500 (M+H); ¹H NMR (400 MHz, Methanol-d₄) δ ppm 1.47-1.57 (m, 2H), 1.71 (s, 3H), 1.72 (s, 3H), 1.78-1.98 (m, 8H), 2.01-2.17 (m, 2H), 3.11 (br. s., 1H), 3.98 (s, 3H), 4.05 (br. s., 1H), 5.07-5.18 (m, 1H), 7.20 (d, J=8.59 Hz, 1H), 7.54 (d, J=5.91 Hz, 1H), 7.72-7.79 (m, 2H), 8.25-8.34 (m, 1H), 8.37-8.44 (m, 1H), 8.46-8.56 (m, 1H), 8.82-8.91 (m, 1H), 9.27 (s, 1H), 9.42 (s, 1H).

### EXAMPLE 128

### N-Furan-2-ylmethyl-3-[4-(1-isopropyl-1H-pyrrolo[2,3-c]pyridin-3-yl)-pyrimidin-2-ylamino]-4-methoxy-benzamide [128] (Hereinafter Referred to as “Compound [128]”)

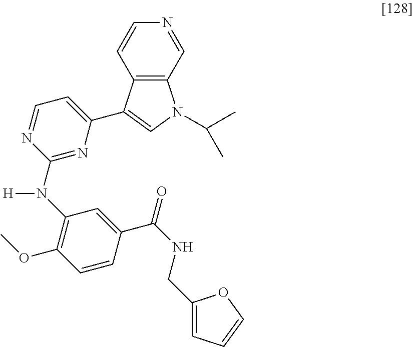

Compound [128] was prepared using a procedure similar to that of Example 127.

Data for Compound [128]: LCMS m/e 483 (M+H); ¹H NMR (400 MHz, Methanol-d₄) δ ppm 1.70 (s, 3H), 1.72 (s, 3H), 3.99 (s, 3H), 4.53 (s, 2H), 5.05-5.15 (m, 1H), 6.24 (d, J=3.03 Hz, 1H), 6.29-6.34 (m, 1H), 7.16 (d, J=8.59 Hz, 1H), 7.36 (d, J=0.93 Hz, 1H), 7.46 (d, J=5.56 Hz, 1H), 7.67 (dd, J=8.52, 2.17 Hz, 1H), 8.19 (d, J=6.49 Hz, 1H), 8.43 (d, J=5.52 Hz, 1H), 8.74 (d, J=2.15 Hz, 1H), 8.89 (d, J=6.49 Hz, 1H), 9.20 (s, 2H), 9.37 (s, 1H).

### EXAMPLE 129

### N-Cyclopentyl-3-[4-(1-isopropyl-1H-pyrrolo[2,3-c]pyridin-3-yl)-pyrimidin-2-ylamino]-4-methoxy-benzamide [129] (Hereinafter Referred to as “Compound [129]”)

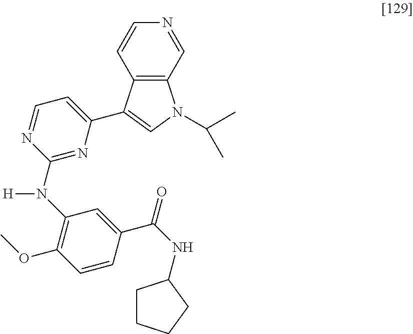

Compound [129] was prepared using a procedure similar to that of Example 127.

Data for Compound [129]: LCMS m/e 471 (M+H); ¹H NMR (400 MHz, Methanol-d₄) δ ppm 1.45-1.57 (m, 2H), 1.57-1.65 (m, 2H), 1.72 (d, J=6.69 Hz, 6H), 1.87-2.02 (m, 2H), 3.98 (s, 3H), 4.23-4.34 (m, 1H), 5.02-5.18 (m, 1H), 7.17 (d, J=8.59 Hz, 1H), 7.49 (d, J=5.71 Hz, 1H), 7.68 (dd, J=8.54, 2.10 Hz, 1H), 8.26 (d, J=6.44 Hz, 1H), 8.42 (d, J=5.66 Hz, 1H), 8.62 (d, J=1.81 Hz, 1H), 8.89 (d, J=6.44 Hz, 1H), 9.23 (s, 1H), 9.40 (s, 1H).

### EXAMPLE 130

### 3-[4-(1-Isopropyl-1H-pyrrolo[2,3-c]pyridin-3-yl)-pyrimidin-2-ylamino]-4-methoxy-N-[2-(1-methyl-pyrrolidin-2-yl)-ethyl]-benzamide [130] (Hereinafter Referred to as “Compound [130]”)

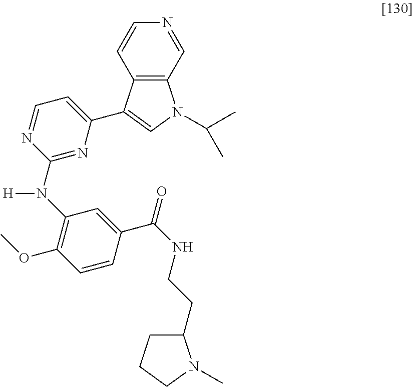

Compound [130] was prepared using a procedure similar to that of Example 127.

Data for Compound [130]: LCMS m/e 514 (M+H); ¹H NMR (400 MHz, Methanol-d₄) δ ppm 1.72 (d, J=6.69 Hz, 6H), 1.81-1.90 (m, 2H), 1.99-2.18 (m, 2H), 2.20-2.33 (m, 1H), 2.40-2.55 (m, 1H), 2.88-2.95 (m, 4H), 3.09-3.20 (m, 1H), 3.45-3.54 (m, 2H), 3.61-3.71 (m, 1H), 4.00 (s, 3H), 5.04-5.20 (m, 1H), 7.16 (d, J=8.59 Hz, 1H), 7.45 (d, J=5.47 Hz, 1H), 7.64 (dd, J=8.52, 2.12 Hz, 1H), 8.30 (d, J=6.49 Hz, 1H), 8.46 (d, J=5.47 Hz, 1H), 8.75 (d, J=1.51 Hz, 1H), 8.96 (d, J=6.49 Hz, 1H), 9.19 (s, 1H), 9.40 (s, 1H).

### EXAMPLE 131

### N-Isopropyl-3-[4-(1-isopropyl-1H-pyrrolo[2,3-c]pyridin-3-yl)-pyrimidin-2-ylamino]-4-methoxy-benzamide [131] (Hereinafter Referred to as “Compound [131]”)

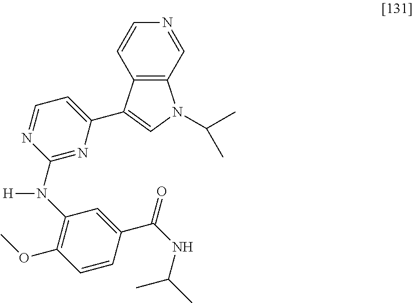

Compound [131] was prepared using a procedure similar to that of Example 127.

Data for Compound [131]: LCMS m/e 445 (M+H); ¹H NMR (400 MHz, Methanol-d₄) δppm 1.21 (d, J=6.64 Hz, 6H), 1.72 (d, J=6.64 Hz, 6H), 3.98 (s, 3H), 4.12-4.24 (m, 1H), 5.05-5.21 (m, 1H), 7.18 (d, J=8.59 Hz, 1H), 7.50 (d, J=5.71 Hz, 2H), 7.69 (dd, J=8.54, 2.00 Hz, 1H), 8.26 (d, J=6.49 Hz, 1H), 8.42 (d, J=5.61 Hz, 1H), 8.56-8.61 (m, 1H), 8.88 (d, J=6.10 Hz, 2H), 9.24 (s, 1H), 9.41 (s, 1H).

### EXAMPLE 132

### N-(2-Acetylamino-ethyl)-3-[4-(1-isopropyl-1H-pyrrolo[2,3-c]pyridin-3-yl)-pyrimidin-2-ylamino]-4-methoxy-benzamide [132] (Hereinafter Referred to as “Compound [132]”)

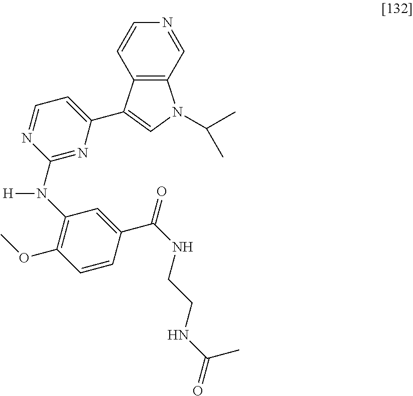

Compound [132] was prepared using a procedure similar to that of Example 127.

Data for Compound [132]: LCMS m/e 488 (M+H); ¹H NMR (400 MHz, Methanol-d₄) δ ppm 1.71 (s, 3H), 1.72 (s, 3H), 1.92 (s, 3H), 3.33-3.38 (m, 2H), 3.46 (t, J=6.00 Hz, 2H), 3.99 (s, 3H), 5.03-5.19 (m, 1H), 7.15 (d, J=8.54 Hz, 1H), 7.45 (d, J=5.47 Hz, 1H), 7.62 (dd, J=8.52, 2.12 Hz, 1H), 7.88 (s, 1H), 8.29 (d, J=6.49 Hz, 1H), 8.45 (d, J=5.47 Hz, 1H), 8.76 (d, J=2.00 Hz, 1H), 8.93 (d, J=6.49 Hz, 1H), 9.19 (s, 1H), 9.38 (s, 1H).

### EXAMPLE 133

### N-(4-Hydroxy-cyclohexyl)-3-[4-(1-isopropyl-1H-pyrrolo[2,3-c]pyridin-3-yl)-pyrimidin-2-ylamino]-4-methoxy-benzamide [133] (Hereinafter Referred to as “Compound [133]”)

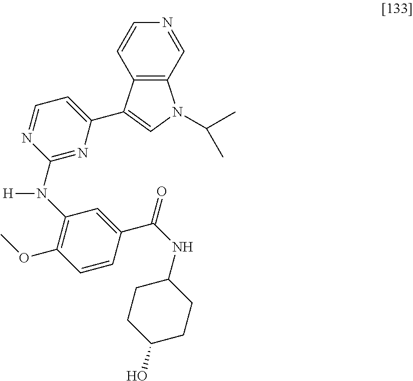

Compound [133] was prepared using a procedure similar to that of Example 127.

Data for Compound [133]: LCMS m/e 501 (M+H); ¹H NMR (400 MHz, Methanol-d₄) δ ppm 1.30-1.48 (m, 4H), 1.71 (s, 3H), 1.72 (s, 3H), 1.87-2.02 (m, 4H), 3.54 (t, J=9.86 Hz, 1H), 3.82 (t, J=10.71 Hz, 1H), 3.98 (s, 3H), 5.03-5.18 (m, 1H), 7.18 (d, J=8.64 Hz, 1H), 7.51 (d, J=5.81 Hz, 1H), 7.70 (dd, J=8.57, 2.17 Hz, 1H), 8.26 (d, J=6.49 Hz, 1H), 8.41 (d, J=5.76 Hz, 1H), 8.57 (s, 1H), 8.86 (d, J=6.39 Hz, 1H), 9.25 (s, 1H), 9.41 (s, 1H).

### EXAMPLE 134

### 3-[4-(1-Isopropyl-1H-pyrrolo[2,3-c]pyridin-3-yl)-pyrimidin-2-ylamino]-4-methoxy-N-(2-methylsulfanyl-ethyl)-benzamide [134] (Hereinafter Referred to as “Compound [134]”)

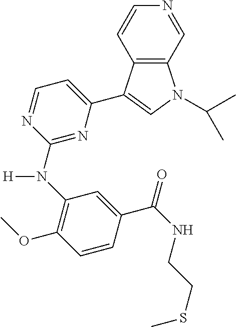

Compound [134] was prepared using a procedure similar to that of Example 127.

Data for Compound [134]: LCMS m/e 477 (M+H); ¹H NMR (400 MHz, Methanol-d₄) δ ppm 1.71 (s, 3H), 1.72 (s, 3H), 2.11 (s, 3H), 2.66 (t, J=7.00 Hz, 2H), 3.55 (t, J=6.98 Hz, 2H), 3.99 (s, 3H), 5.06-5.18 (m, 1H), 7.17 (d, J=8.54 Hz, 1H), 7.46 (d, J=5.52 Hz, 1H), 7.65 (dd, J=8.54, 2.05 Hz, 1H), 8.26 (d, J=6.49 Hz, 1H), 8.45 (d, J=5.56 Hz, 1H), 8.72 (s, 1H), 8.89 (d, J=6.49 Hz, 1H), 9.20 (s, 1H), 9.39 (s, 1H).

### EXAMPLE 135

### N-Cyanomethyl-3-[4-(1-isopropyl-1H-pyrrolo[2,3-c]pyridin-3-yl)-pyrimidin-2-ylamino]-4-methoxy-benzamide [135] (Hereinafter Referred to as “Compound [135]”)

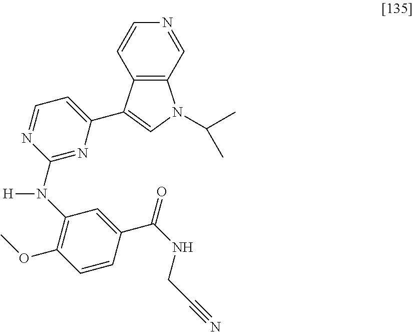

Compound [135] was prepared using a procedure similar to that of Example 127.

Data for Compound [135]: LCMS m/e 442 (M+H); ¹H NMR (400 MHz, Methanol-d₄) δ ppm 1.72 (d, J=6.69 Hz, 6H), 4.00 (s, 3H), 4.28 (s, 2H), 4.98-5.22 (m, 1H), 7.19 (d, J=8.59 Hz, 1H), 7.49 (d, J=5.56 Hz, 1H), 7.67 (dd, J=8.54, 2.20 Hz, 1H), 8.30 (d, J=6.49 Hz, 1H), 8.46 (d, J=5.56 Hz, 1H), 8.84 (d, J=2.10 Hz, 1H), 8.91 (d, J=6.49 Hz, 1H), 9.23 (s, 1H), 9.38 (s, 1H).

### EXAMPLE 136

### {3-[4-(1-Isopropyl-1H-pyrrolo[2,3-c]pyridin-3-yl)-pyrimidin-2-ylamino]-4-methoxy-benzoylamino}-acetic acid [136] (Hereinafter Referred to as “Compound [136]”)

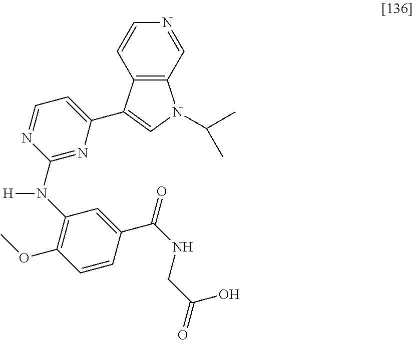

Compound [136] was prepared using a procedure similar to that of Example 127 except the crude was stirred with TFA (2 mL) for 2 hours and concentrated and purified on prep-HPLC.

Data for Compound [136]: LCMS m/e 461 (M+H); ¹H NMR (400 MHz, Methanol-d₄) δppm 1.71 (d, J=6.69 Hz, 6H), 4.00 (s, 3H), 4.08 (s, 2H), 5.02-5.15 (m, 1H), 7.17 (d, J=8.59 Hz, 1H), 7.45 (d, J=5.52 Hz, 1H), 7.69 (dd, J=8.52, 2.12 Hz, 1H), 8.28 (d, J=6.49 Hz, 1H), 8.44 (d, J=5.47 Hz, 1H), 8.79 (d, J=2.10 Hz, 1H), 8.88 (d, J=6.44 Hz, 1H), 9.19 (s, 1H), 9.35 (s, 1H).

### EXAMPLE 137

### 3-[4-(1-Isopropyl-1H-pyrrolo[2,3-c]pyridine-3-yl)-pyrimidin-2-ylamino]-4-methoxy-benzoic acid [137] (Hereinafter Referred to as “Compound [137]”)

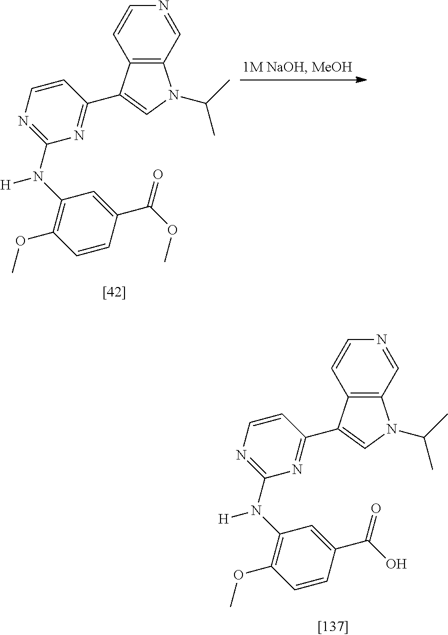

Compound [42] (101.9 mg, 241.9 mmol, 1 eq.) was dissolved in 2 mL of methanol. To this was added 2 mL of 1M NaOH (aqueous), and the reaction was stirred under N₂ atmosphere for 18 hours. The reaction was then neutralized with glacial acetic acid and the reaction mixture was concentrated under reduced pressure onto Celite. The residue was purified by silica gel chromatography (gradient 1% MeOH to 10% MeOH in DCM) to provide Compound [137].

Data for Compound [137]: LC/MS m/e 404 (M+H); ¹H NMR (400 MHz, DMSO-d₆) δ ppm 1.57 (s, 3H), 1.59 (s, 3H), 3.95 (s, 3H), 5.01 (quin, 1H), 7.17 (d, J=8.6 Hz, 1H), 7.39 (d, J=5.4 Hz, 1H), 7.70 (dd, J=8.5, 2.0 Hz, 1H), 8.15 (s, 1H), 8.22 (d, J=5.5 Hz, 1H), 8.30 (d, J=5.4 Hz, 1H), 8.42 (d, J=5.3 Hz, 1H), 8.69 (s, 1H), 8.84 (d, J=2.0 Hz, 1H), 9.02 (s, 1H).

### EXAMPLE 138

### N-3-[4-(1-Isopropyl-1H-pyrrolo[2,3-c]pyridin-3-yl)-pyrimidin-2-yl]-4-methoxy-benzene-1,3-diamine [138] (Hereinafter Referred to as “Compound [138]”)

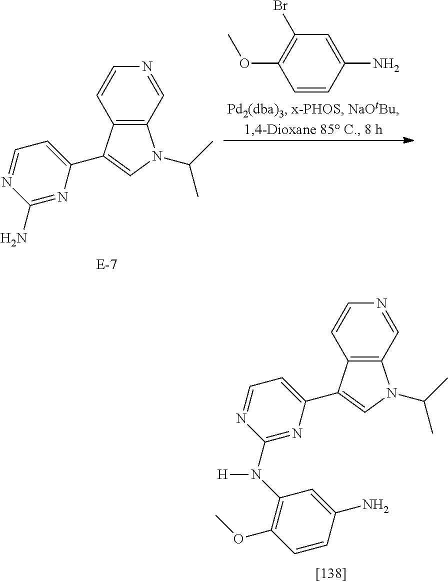

To a 40 mL scintillation vial fitted with a magnetic stir bar was added the starting material E-7 (0.212 g, 0.837 mmol, 1 eq.) and N₂ was bubbled through the vessel for 10 minutes. To the vessel was added 1,4-dioxane (5 mL) followed by the addition of 3-Bromo-4-methoxy aniline (0.185 g, 0.920 mmol, 1.1 eq.) and N₂ bubbled through the solution for an additional 5 minutes. X-PHOS (0.079 g, 0.167 mmol, 0.2 eq.), sodium tert-butoxide (0.240 g, 2.51 mmol, 3 eq.), and Pd₂(dba)₃ (0.0375 g, 0.041 mmol, 0.05 eq.) were then added sequentially. The reaction was stirred 16 hours under N₂ at 85° C. The reaction mixture was then cooled, concentrated to dryness, and the residue re-dissolved in methanol (5 mL), filtered through a 0.2 micron syringe filter, and purified by reverse-phase preparatory HPLC using acetonitrile and water with 0.05% TFA as the eluent to provide Compound [138].

Data for Compound [138]: LCMS m/e 375 (M+H) ¹H NMR (400 MHz, Methanol-d₄) δppm 1.71 (d, J=6.69 Hz, 6H), 4.01 (s, 3H), 5.02-5.22 (m, 1H), 7.05 (dd, J=8.61, 2.66 Hz, 1H), 7.17 (d, J=8.69 Hz, 1H), 7.48 (d, J=5.42 Hz, 1H), 8.39 (d, J=6.49 Hz, 1H), 8.52 (d, J=5.37 Hz, 1H), 8.62 (d, J=2.64 Hz, 1H), 9.05 (d, J=6.49 Hz, 1H), 9.16 (s, 1H), 9.42 (s, 1H).

### EXAMPLE 139

### 4-Amino-2-[4-(1-isopropyl-1H-pyrrolo[2,3-c]pyridin-3-yl)-pyrimidin-2-ylamino]-pyrimidine-5-carbonitrile [139] (Hereinafter Referred to as “Compound [139]”)

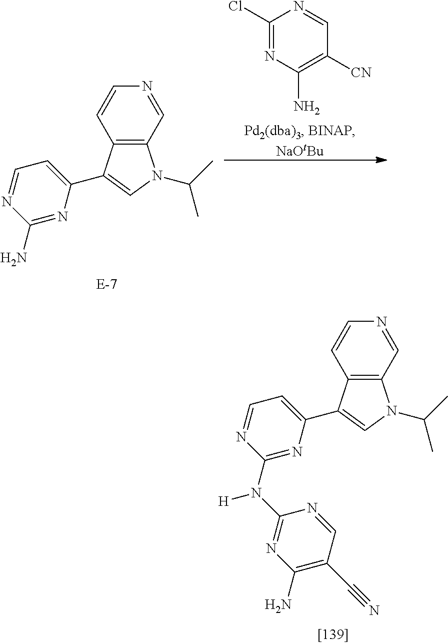

Compound E-7 (65 mg, 0.26 mmol, 1.0 eq), Pd₂(dba)₃ (23 mg, 0.026 mmol, 0.10 eq), BINAP (32 mg, 0.05, 0.20 eq), NaOtBu (74 mg, 0.77 mmol, 3.0 eq), and the 2-amino-1-chloropyrimidine-5-nitrile (79 mg, 0.51 mmol, 2.0 eq) were dissolved in dioxane (1.5 mL, degassed, anhydrous). The reaction was heated to 100° C. for 16 hours. The resulting solution was cooled, added CHCl₃ and MeOH, concentrated onto Celite, and purified by silica gel chromatography (gradient of CHCl₃ to CHCl₃/MeOH/NH₄Cl 90:10:1). Further purification by reverse-phase chromatography (Solvent A H₂O/CH₃CN/TFA (95:5:0.05), Solvent B CH₃CN/H₂O/TFA (95:5:0.05)) with a gradient of 10% to 80% B over 5 min gave Compound [139].

Data for Compound [139]: LCMS 372 (M+H); ¹H NMR (400 MHz, Methanol-d₄) δ ppm 1.74 (d, J=6.69 Hz, 6H), 5.14 (spt, 1H), 6.30 (d, J=1.12 Hz, 1H), 6.35 (br. s., 1H), 7.67 (d, J=5.52 Hz, 1H), 8.42 (d, J=6.54 Hz, 1H), 8.61 (d, J=5.52 Hz, 1H), 9.17 (d, J=6.44 Hz, 1H), 9.24 (s, 1H), 9.45 (s, 1H).

### EXAMPLE 140

### 4-Amino-2-[4-(1-isopropyl-1H-pyrrolo[2,3-c]pyridin-3-yl)-pyrimidin-2-ylamino]-pyrimidine-5-carboxylic acid amide [140] (Hereinafter Referred to as “Compound [140]”)

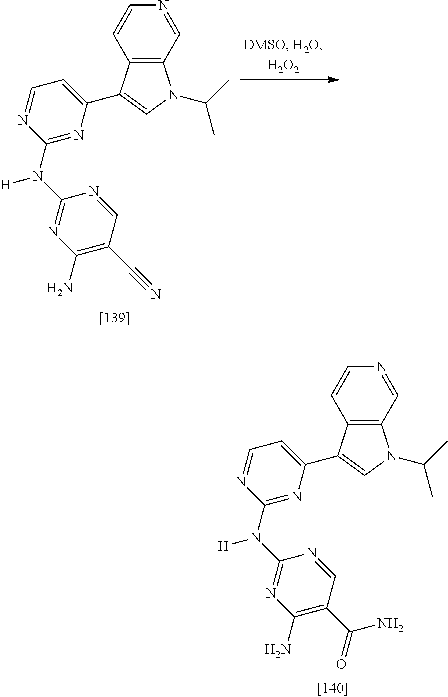

Compound [139] (75 mg, 0.20 mmol, 1.0 eq) was dissolved in DMF (1 mL) and then was added NaOH (aqueous 5.0 N, 110 μL), H₂O (110 μL), H₂O₂ (30%, 220 μL), and DMSO (100 μL). The reaction was heated to 50° C. for 15 minutes, and was cooled, added H₂O, and extracted with CHCl₃ three times. The combined organics were dried and concentrated. Then purified by silica gel chromatography (gradient of CHCl₃ to CHCl₃/MeOH/NH₄Cl 90:10:1). Further purification by reverse-phase chromatography (Solvent A H₂O/CH₃CN/TFA (95:5:0.05), Solvent B CH₃CN/H₂O/TFA (95:5:0.05)) with a gradient of 10% to 60% B over 5 minutes provided Compound [140].

Data for Compound [140]: LCMS 390 (M+H); ¹H NMR (400 MHz, Methanol-d₄) δ ppm 1.74 (d, J=6.30 Hz, 6H), 5.05-5.20 (m, 1H), 6.36 (d, J=0.59 Hz, 1H), 6.47 (s, 1H), 7.64 (d, J=5.37 Hz, 1H), 8.40 (d, J=6.15 Hz, 1H), 8.55 (d, J=5.47 Hz, 1H), 9.18 (d, J=6.39 Hz, 1H), 9.22 (s, 1H), 9.42 (s, 1H).

### EXAMPLE 141

### 2-[4-(1-Isopropyl-1H-pyrrolo[2,3-c]pyridin-3-yl)-pyrimidin-2-ylamino]-isonicotinonitrile [141] (Hereinafter Referred to as “Compound [141]”)

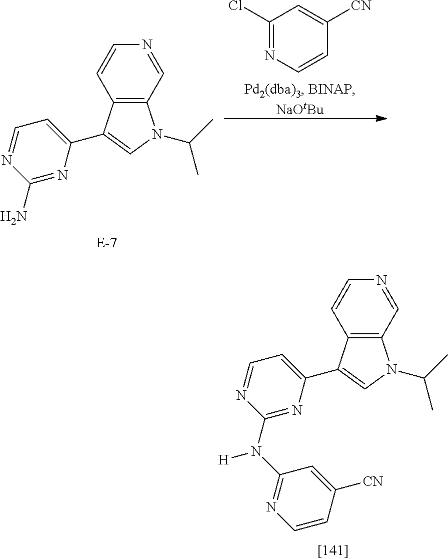

Compound E-7 (65 mg, 0.26 mmol, 1.0 eq), Pd₂(dba)₃ (23 mg, 0.026 mmol, 0.10 eq), BINAP (32 mg, 0.05, 0.20 eq), NaOtBu (74 mg, 0.77 mmol, 3.0 eq), and 2-chloropyridine-4-nitrile (71 mg, 0.51 mmol, 2.0 eq) were dissolved in dioxane (1.5 mL, degassed, anhydrous). The reaction was heated to 100° C. for 16 hours. The mixture was then cooled, CHCl₃ and MeOH added, concentrated onto Celite, and purified by silica gel chromatography (gradient of CHCl₃ to CHCl₃/MeOH/NH₄Cl 90:10:1). Further purification by reverse-phase chromatography (Solvent A H₂O/CH₃CN/TFA (95:5:0.05), Solvent B CH₃CN/H₂O/TFA (95:5:0.05)) with a gradient of 10% to 80% B over 5 min provided Compound [141].

Data for Compound [141]: LCMS 356 (M+H); ¹H NMR (400 MHz, Methanol-d₄) δ ppm 1.74 (d, J=6.69 Hz, 6H), 5.15 (spt, 1H), 7.37 (dd, J=5.12, 1.32 Hz, 1H), 7.68 (d, J=5.71 Hz, 1H), 8.45 (d, J=6.44 Hz, 1H), 8.47-8.49 (m, 1H), 8.55 (dd, J=5.12, 0.63 Hz, 1H), 8.62 (d, J=5.71 Hz, 1H), 9.23 (d, J=6.49 Hz, 1H), 9.27 (s, 1H), 9.46 (s, 1H).

### EXAMPLE 142

### 2-[4-(1-Isopropyl-1H-pyrrolo[2,3-c]pyridin-3-yl)-pyrimidin-2-ylamino]-isonicotinamide [142] (Hereinafter Referred to as “Compound [142]”)

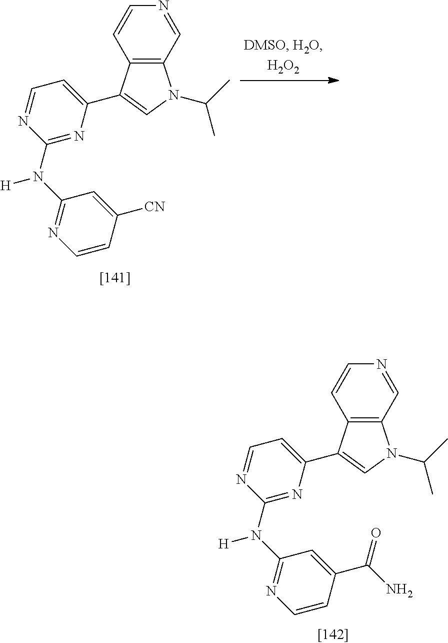

To a solution of Compound [141] (107 mg, 0.30 mmol, 1.0 eq) in DMF (1 mL) was added NaOH (aqueous 5.0 N, 110 μL), H₂O (110 μL), H₂O₂ (30%, 220 μL), and DMSO (100 μL). The reaction was heated to 50° C. for 15 minutes, and was cooled, added H₂O, and extracted with CHCl₃ (three times). The combined organics were dried and concentrated, then purified by silica gel chromatography (gradient of CHCl₃ to CHCl₃/MeOH/NH₄Cl 90:10:1). Further purification by reverse-phase chromatography (Solvent A H₂O/CH₃CN/TFA (95:5:0.05), Solvent B CH₃CN/H₂O/TFA (95:5:0.05)) with a gradient of 10% to 60% B over 5 minutes afforded Compound [142].

Data for Compound [142]: LCMS 374 (M+H); ¹H NMR (400 MHz, Methanol-d₄) δ ppm 1.74 (d, J=6.69 Hz, 6H), 5.16 (spt, J=6.67 Hz, 1H), 7.64 (dd, J=5.93, 1.54 Hz, 1H), 7.85 (d, J=5.91 Hz, 1H), 8.09 (s, 1H), 8.48 (d, J=6.49 Hz, 1H), 8.53 (d, J=5.95 Hz, 1H), 8.68 (d, J=5.91 Hz, 1H), 9.27 (d, J=6.49 Hz, 1H), 9.35 (s, 1H), 9.49 (s, 1H).

### EXAMPLE 143

### [4-(1-Isopropyl-1H-pyrrolo[2,3-c]pyridin-3-yl)-pyrimidin-2-yl]-(4-methyl-thiazol-2-yl)-amine [143-1] (Hereinafter Referred to as “Compound [143-1]”) and [4-(7-Chloro-1-isopropyl-1H-pyrrolo[2,3-c]pyridin-3-yl)-pyrimidin-2-yl]-(4-methyl-thiazol-2-yl)-amine [143-2] (Hereinafter Referred to as “Compound [143-2]”)

### (1) 5-Bromo-4-methyl-thiazol-2-yl)-[4-(7-chloro-1-isopropyl-1H-pyrrolo[2,3-c]pyridin-3-yl)-pyrimidin-2-yl]-amine (CVI)

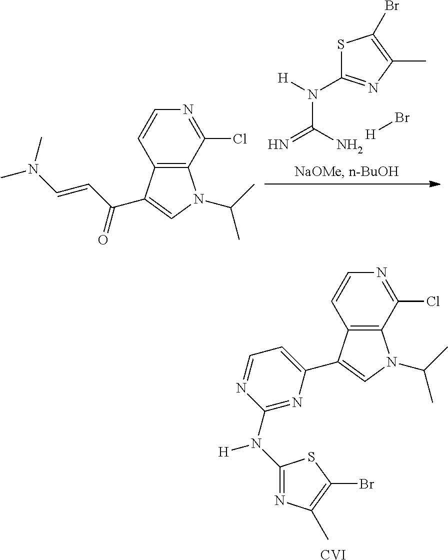

N-(5-Bromo-4-methyl-thiazol-2-yl)-guanidine hydrobromide (162 mg, 0.51 mmol, 2.0 eq) was stirred in n-butanol (2 mL), and NaOMe (56 mg, 1.03 mmol, 4.0 eq) and stirred at room temperature for 30 minutes. Then, a solution of Compound E-5 (75 mg, 0.26 mmol, 1.0 eq) in n-butanol (2 mL) was added and the resulting solution was heated to 110° C. for 6 hours. The reaction was then cooled and concentrated. To the crude solid was added water and extracted with CHCl₃ (three times). The combined organic phases were dried, concentrated, and purified by silica gel chromatography (gradient of CHCl₃ to CHCl₃/MeOH/NH₄Cl 90:10:1) to give Compound CVI. LCMS 479 (M+H). The compound was used in the next step without further characterization.

### (2) [4-(1-Isopropyl-1H-pyrrolo[2,3-c]pyridin-3-yl)-pyrimidin-2-yl]-(4-methyl-thiazol-2-yl)-amine [143-1] and [4-(7-Chloro-1-isopropyl-1H-pyrrolo[2,3-c]pyridin-3-yl)-pyrimidin-2-yl]-(4-methyl-thiazol-2-yl)-amine [143-2]

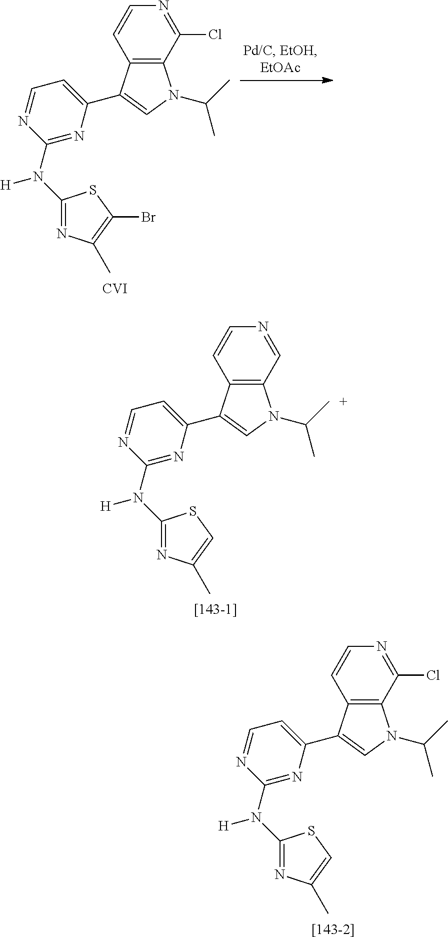

Compound CVI (72 mg, 0.155 mmol) was dissolved in EtOH (2 mL) and EtOAc (2 mL). The solution was degassed, added 10% Pd/C (15 mg), and bubbled through H₂ from a balloon. After 24 hours at room temperature, the reaction was filtered through Celite and concentrated. The residue was purified by reverse-phase chromatography (Solvent A H₂O/CH₃CN/TFA (95:5:0.05), Solvent B CH₃CN/H₂O/TFA (95:5:0.05)) with a gradient of 10% to 60% B over 5 minutes to give Compound [143-1] and Compound [143-2].

Data for Compound [143-1]: LCMS 351 (M+H); ¹H NMR (400 MHz, Methanol-d₄) δ ppm 1.74 (d, J=6.69 Hz, 6H), 2.40 (s, 3H), 5.15 (dt, J=13.34, 6.63 Hz, 1H), 6.78 (d, J=1.07 Hz, 1H), 7.67 (d, J=5.66 Hz, 1H), 8.40 (d, J=6.54 Hz, 1H), 8.65 (d, J=5.61 Hz, 1H), 9.22-9.31 (m, 2H), 9.46 (s, 1H).

Data for Compound [143-2]: LCMS 385 (M+H); ¹H NMR (400 MHz, Methanol-d₄) δ ppm 1.67 (d, J=6.64 Hz, 6H), 2.37 (s, 3H), 5.76 (quin, J=6.65 Hz, 1H), 6.76 (d, J=1.12 Hz, 1H), 7.56 (d, J=6.15 Hz, 1H), 8.10 (d, J=5.52 Hz, 1H), 8.43 (d, J=6.15 Hz, 1H), 8.55 (d, J=5.42 Hz, 1H), 8.78 (s, 1H).

What is claimed is:

1. A compound of Formula I: 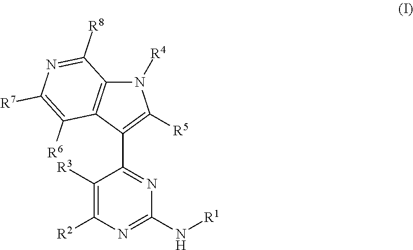
wherein:
R¹ is C₁₋₆ alkyl, C₃₋₈ cycloalkyl, aryl, heterocyclyl, or —COR^{1x},
where the C₁₋₆ alkyl, C₃₋₈ cycloalkyl, aryl, and heterocyclyl may be substituted; and R^{1x} is C₃₋₈ cycloalkyl, aryl, or heterocyclyl, any of which may be substituted;
R², R³, R⁴, R⁵, R⁶, and R⁷ are each independently hydrogen, halogen, C₁₋₆ alkyl, or aryl,
where the C₁₋₆ alkyl or aryl may be substituted;
R⁸ is hydrogen, C₁₋₆ alkyl, aryl, or heterocyclyl, any of which may be substituted;
or a pharmaceutically acceptable salt or ester thereof.

2. The compound according to claim 1 or a pharmaceutically acceptable salt or ester thereof, wherein:
R¹ is aryl, heterocyclyl or —COR^{1x},
where R^{1x} is aryl or heterocyclyl; and
the aryl or heterocyclyl of R¹ and R^{1x} each independently may be substituted with one or more of the same or different substituents selected from:
(i) a substituent selected from L₁;
(ii) C₁₋₆ alkyl which may be substituted with one or more of the same or different substituents selected from L₂;
(iii) —C₀₋₆ alkylene-aryl;
(iv) —C₀₋₆ alkylene-heterocyclyl;
(v) —OR^{a1};
(vi) —O-J-R^{a2};
(vii) —SR^{a1};
(viii) —S-J-R^{a2};
(ix) —NR^{a3}R^{a4};
(x) —CONR^{a3}R^{a4};
(xi) —SO₂R^{a5};
(xii) —SO₂NR^{a3}R^{a4}; and
(xiii) —COOR^{a5} where:
(a1) the —C₀₋₆ alkylene-aryl and —C₀₋₆ alkylene-heterocyclyl each independently may be substituted with one or more of the same or different substituents selected from:
(a1-1) a substituent selected from L₂; and
(a1-2) C₁₋₆ alkyl which may be substituted with one or more of the same or different substituents selected from L₂;
(a2) J is —(CR^{y1}R^{y2})—, —(CR^{y1}R^{y2})—(CR^{y3}R^{y4})—, or —(CR^{y1}R^{y2})—(CR^{y3}R^{y4})—(CR^{y5}R^{y6})—,
where R^{y1}, R^{y2}, R^{y3}, R^{y4}, R^{y5}, and R^{y6} are each independently hydrogen, halogen, hydroxy, cyano, or C₁₋₃ alkyl;
(a3) R^{a1}, R^{a2}, and R^{a5} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
(a3-1) a substituent selected from L₂; and
(a3-2) C₁₋₆ alkyl which may be substituted with one or more of the same or different substituents selected from L₂;
(a3-3) —C₀₋₆ alkylene-aryl which may be substituted with one or more of the same or different substituents selected from L₂;
(a3-4) —C₀₋₆ alkylene-heterocyclyl which may be substituted with one or more of the same or different substituents selected from L₂;
(a4-1) R^{a3} and R^{a4} are each independently hydrogen; C₁₋₆ alkyl which may be substituted with one or more of the same or different substituents selected from L₂; —C₀₋₆ alkylene-C₃₋₈ cycloalkyl; —C₀₋₆ alkylene-aryl; or —C₀₋₆ alkylene-heterocyclyl,
where the —C₀₋₆ alkylene-C₃₋₈ cycloalkyl, —C₀₋₆ alkylene-aryl, and —C₀₋₆ alkylene-heterocyclyl each independently may be substituted with one or more of the same or different substituents selected from:
(a4-1-1) a substituent selected from L₂; and
(a4-1-2) C₁₋₆ alkyl which may be substituted with one or more of the same or different substituents selected from L₂;
(a4-1-3) —C₀₋₆ alkylene-aryl which may be substituted with one or more of the same or different substituents selected from L₂;
(a4-1-4) —C₀₋₆ alkylene-heterocyclyl which may be substituted with one or more of the same or different substituents selected from L₂;
(a4-2) or alternatively, R^{a3} and R^{a4}, together with the nitrogen atom which they are attached to, may form a 5-membered or 6-membered heterocycle,
where the heterocycle is selected from pyrrolidinyl, piperidinyl, piperazinyl, and morpholinyl, and may be substituted with one or more of the same or different substituents selected from L₃;
R², R³, R⁴, R⁵, R⁶, and R⁷ are each independently hydrogen, halogen, C₁₋₆ alkyl, or aryl,
where the C₁₋₆ alkyl or aryl each independently may be substituted with one or more of the same or different substituents selected from L₂;
R⁸ is hydrogen, C₁₋₆ alkyl, aryl, or heterocyclyl,
where the C₁₋₆ alkyl, aryl, and heterocyclyl each independently may be substituted with one or more of the same or different substituents selected from L₂;
L₁ is halogen, cyano, or nitro;
L₂ is halogen, hydroxy, nitro, cyano, amino, carbamoyl, aminosulfonyl, imino, C₁₋₆ alkylamino, di-(C₁₋₆ alkyl)amino, C₁₋₆ alkylsulfonyl, C₁₋₆ alkylsulfonylamino, C₁₋₆ alkoxy, C₁₋₆ alkoxycarbonyl, C₁₋₆ alkoxycarbonylamino, C₁₋₆ alkanoyl, C₁₋₆ alkanoylamino, C₁₋₆ alkanoyloxy, C₁₋₆ alkylthio, or carboxyl; and
L₃ is halogen, hydroxy, or amino

3. The compound according to claim 2 or a pharmaceutically acceptable salt or ester thereof, wherein R², R³, R⁵, R⁶, and R⁷ are each hydrogen.

4. The compound according to claim 3 or a pharmaceutically acceptable salt or ester thereof, wherein R⁸ is hydrogen.

5. The compound according to claim 4 or a pharmaceutically acceptable salt or ester thereof, wherein R⁴ is C₁₋₆ alkyl.

6. The compound according to claim 5 or a pharmaceutically acceptable salt or ester thereof, wherein:
R¹ is phenyl, or heterocyclyl selected from furanyl, pyrrolyl, thienyl, pyridinyl, pyrazinyl, pyridazinyl, pyrimidinyl, thiazolyl, isothiazolyl, oxazolyl, oxadiazolyl, isoxazolyl, imidazolyl, pyrazolyl, and triazolyl, any of which may be substituted with one to three of the same or different substituents selected from:
(i) a substituent selected from L₁;
(ii) C₁₋₆ alkyl which may be substituted with one to three of the same or different substituents selected from L₂ₐ;
(iii) phenyl;
(iv) —C₁₋₃ alkylene-heterocyclyl, the heterocyclyl of which is selected from pyrrolidinyl, piperidinyl, piperazinyl, and morpholinyl;
(v) —OR^{a1};
(vi) —SR^{a1};
(vii) —NR^{a3}R^{a4};
(viii) —CONR^{a3}R^{a4};
(ix) —SO₂NR^{a3}R^{a4}; and
(x) —COOR^{a5} where:
(aa1) the phenyl and —C₁₋₃ alkylene-heterocyclyl each independently may be substituted with one to three of the same or different substituents selected from:
(aa1-1) a substituent selected from L_{2b}; and
(aa1-2) C₁₋₆ alkyl which may be substituted with one to three of the same or different substituents selected from L_{2b};
(aa2) R^{a1} and R^{a5} are each independently hydrogen; C₁₋₆ alkyl which may be substituted with one to three of the same or different substituents selected from L_{2b}; or —C₀₋₆ alkylene-phenyl which may be substituted with one to three of the same or different substituents selected from:
(aa2-1) a substituent selected from L_{2b}; and
(aa2-2) C₁₋₆ alkyl which may be substituted with one to three of the same or different substituents selected from L_{2b};
(aa3-1) R^{a3} and R^{a4} are each independently hydrogen; C₁₋₆ alkyl which may be substituted with one to three of the same or different substituents selected from L_{2c}; —C₀₋₆ alkylene-C₅₋₆ cycloalkyl; —C₁₋₆ alkylene-phenyl; and —C₁₋₆ alkylene-heterocyclyl,
where the heterocyclyl of —C₁₋₆ alkylene-heterocyclyl is a 5-membered or 6-membered aliphatic or aromatic heterocycle, and
the —C₀₋₆ alkylene-C₅₋₆ cycloalkyl, —C₁₋₆ alkylene-aryl, and —C₁₋₆ alkylene-heterocyclyl each independently may be substituted with one to three of the same or different substituents selected from:
(aa3-1-1) a substituent selected from L_{2c}; and
(aa3-1-2) C₁₋₆ alkyl which may be substituted with one to three of the same or different substituents selected from L_{2c};
(aa3-2) or alternatively, R^{a3} and R^{a4}, together with the nitrogen atom which they are attached to, may form a 5-membered or 6-membered aliphatic heterocycle, where the aliphatic heterocycle is selected from pyrrolidinyl, piperidinyl, piperazinyl, and morpholinyl, and may be substituted with one to three of the same or different substituents selected from L₃;
L₂ₐ is halogen, hydroxy, carbamoyl, or carboxyl;
L_{2b} is halogen or hydroxy;
L_{2c} is halogen, hydroxy, cyano, amino, carbamoyl, C₁₋₆ alkylamino, di-(C₁₋₆ alkyl)amino, C₁₋₆ alkoxy, C₁₋₆ alkoxycarbonylamino, C₁₋₆ alkanoyl, C₁₋₆ alkanoylamino, C₁₋₆ alkanoyloxy, C₁₋₆ alkylthio, or carboxyl
L₃ is halogen, hydroxy, or amino

7. The compound according to claim 6 or a pharmaceutically acceptable salt or ester thereof, wherein R⁴ is isopropyl.

8. The compound according to claim 7 or a pharmaceutically acceptable salt or ester thereof, wherein:
R¹ is phenyl disubstituted at the 2- and 5-positions with C₁₋₆ alkyl or —OR^{a1} and with —CONR^{a3}R^{a4}, respectively, where:
(aaa1) R^{a1} is C₁₋₆ alkyl which may be substituted with one to three of the same or different substituents selected from L_{2b}; or —C₀₋₆ alkylene-phenyl which may be substituted with one to three of the same or different substituents selected from:
(aaa1-1) a substituent selected from L_{2b}; and
(aaa1-2) C₁₋₆ alkyl which may be substituted with one to three of the same or different substituents selected from L_{2b};
(aaa2) R^{a3} and R^{a4} are each independently hydrogen; C₁₋₆ alkyl which may be substituted with one to three of the same or different substituents selected from L_{2c}; —C₀₋₃ alkylene-C₅₋₆ cycloalkyl; —C₁₋₃ alkylene-phenyl; or —C₁₋₃ alkylene-heterocyclyl,
where the heterocyclyl of —C₁₋₃ alkylene-heterocyclyl is selected from pyrrolidinyl, piperidinyl, piperazinyl, and morpholinyl, furanyl, pyrrolyl, thienyl, pyridinyl, pyrazinyl, pyridazinyl, pyrimidinyl, thiazolyl, isothiazolyl, oxazolyl, oxadiazolyl, isoxazolyl, and imidazolyl; and the —C₀₋₃ alkylene-C₅₋₆ cycloalkyl; —C₁₋₃ alkylene-phenyl; or —C₁₋₃ alkylene-heterocyclyl each independently may be substituted with one to three of the same or different substituents selected from:
(aaa2-1) a substituent selected from L_{2c}; and
(aaa2-2) C₁₋₆ alkyl which may be substituted with one to three of the same or different substituents selected from L_{2c};
L_{2b} is halogen or hydroxy;
L_{2c} is halogen, hydroxy, cyano, amino, carbamoyl, C₁₋₆ alkylamino, di-(C₁₋₆ alkyl)amino, C₁₋₆ alkoxy, C₁₋₆ alkanoylamino, C₁₋₆ alkylthio, or carboxyl;

9. A compound which is:
(a) N-[4-(1-Isopropyl-1H-pyrrolo[2,3-c]pyridin-3-yl)-pyrimidin-2-yl]-benzamide;
(b) [4-(1-Isopropyl-1H-pyrrolo[2,3-c]pyridin-3-yl)-pyrimidin-2-yl]-phenyl-amine;
(c) 4-[4-(1-Isopropyl-1H-pyrrolo[2,3-c]pyridin-3-yl)-pyrimidin-2-ylamino]-benzenesulfonamide;
(d) 3-[4-(1-Isopropyl-1H-pyrrolo[2,3-c]pyridin-3-yl)-pyrimidin-2-ylamino]-benzenesulfonamide;
(e) 3-[4-(1-Isopropyl-1H-pyrrolo[2,3-c]pyridin-3-yl)-pyrimidin-2-ylamino]-4-methoxy-N-methyl-benzamide;
(f) 3-Bromo-5-[4-(1-isopropyl-1H-pyrrolo[2,3-c]pyridine-3-yl)-pyrimidin-2-ylamino]-benzoic acid;
(g) 5-[4-(1-Isopropyl-1H-pyrrolo[2,3-c]pyridin-3-yl)-pyrimidin-2-ylamino]-2-methyl-benzenesulfonamide;
(h) N-(2-Hydroxy-ethyl)-4-[4-(1-isopropyl-1H-pyrrolo[2,3-c]pyridin-3-yl)-pyrimidin-2-ylamino]-benzamide;
(i) 3-[4-(1-Isopropyl-1H-pyrrolo[2,3-c]pyridin-3-yl)-pyrimidin-2-ylamino]-4-methyl-benzamide;
(j) 3-[4-(1-Isopropyl-1H-pyrrolo[2,3-c]pyridin-3-yl)-pyrimidin-2-ylamino]-N-methyl-benzamide];
(k) 3-Bromo-5-[4-(1-isopropyl-1H-pyrrolo[2,3-c]pyridin-3-yl)-pyrimidin-2-ylamino]-N-methyl-benzamide;
(l) 3-Bromo-5-[4-(1-isopropyl-1H-pyrrolo[2,3-c]pyridin-3-yl)-pyrimidin-2-ylamino]-benzamide;
(m) 3-[4-(1-Isopropyl-1H-pyrrolo[2,3-c]pyridin-3-yl)-pyrimidin-2-ylamino]-5-trifluoromethyl-benzoic acid;
(n) 3-Chloro-5-[4-(1-isopropyl-1H-pyrrolo[2,3-c]pyridin-3-yl)-pyrimidin-2-ylamino]-benzoic acid;
(o) 3-[4-(1-Isopropyl-1H-pyrrolo[2,3-c]pyridine-3-yl)-pyrimidin-2-ylamino]-4-methoxy-benzamide;
(p) 3-[4-(1-Isopropyl-1H-pyrrolo[2,3-c]pyridin-3-yl)-pyrimidin-2-ylamino]-5-methoxy-benzamide;
(q) 4-Benzyloxy-3-[4-(1-isopropyl-1H-pyrrolo[2,3-c]pyridin-3-yl)-pyrimidin-2-ylamino]-benzamide;
(r) 3-[4-(1-Isopropyl-1H-pyrrolo[2,3-c]pyridin-3-yl)-pyrimidin-2-ylamino]-5-trifluoromethyl-benzamide;
(s) 3-[4-(1-Isopropyl-1H-pyrrolo[2,3-c]pyridin-3-yl)-pyrimidin-2-ylamino]-N-methyl-5-trifluoromethyl-benzamide;
(t) 3-[4-(1-Isopropyl-1H-pyrrolo[2,3-c]pyridin-3-yl)-pyrimidin-2-ylamino]-5-trifluoromethoxy-benzamide;
(u) 3-Chloro-5-[4-(1-isopropyl-1H-pyrrolo[2,3-c]pyridin-3-yl)-pyrimidin-2-ylamino]-N-methyl-benzamide;
(v) 3-[4-(1-Isopropyl-1H-pyrrolo[2,3-c]pyridin-3-yl)-pyrimidin-2-ylamino]-5-methyl-benzamide; or
(w) 3-[4-(1-Isopropyl-1H-pyrrolo[2,3-c]pyridin-3-yl)-pyrimidin-2-ylamino]-4-methoxy-benzonitrile,
or a pharmaceutically acceptable salt or ester thereof.

10. A pharmaceutical composition comprising, together with pharmaceutically acceptable carrier or diluent, at least one compound according to claim 1 as active ingredient.

11. A NIK inhibitor comprising, together with a pharmaceutically acceptable carrier or diluent, at least one compound according to claim 1 as active ingredient.

12. An anti-cancer agent comprising, together with a pharmaceutically acceptable carrier or diluent, at least one compound according to claim 1 as active ingredient.
